(19) United States
(12) Patent Application Publication
(10) Pub. No.: US 2017/0342025 A1
(43) Pub. Date: Nov. 30, 2017

(54) ANTIMICROBIAL COMPOUNDS, COMPOSITIONS AND METHODS

(71) Applicants: Swansea University, Singleton Park, Swansea (GB); The University of Bath, Claverton Down, Bath (GB)

(72) Inventors: Barry Victor Lloyd POTTER, Bath, Avon (GB); Wolfgang DOHLE, Bath, Avon (GB); Xiangdong SU, Bath, Avon (GB); John NORMANTON, Bath, Avon (GB); Edward DUDLEY, Swansea, West Glamorgan (GB); Yamni NIGAM, Swansea, West Glamorgan (GB)

(73) Assignees: The University of Bath, Claverton Down, Bath (GB); Swansea University, Singleton Park, Swansea (GB)

(21) Appl. No.: 15/541,113

(22) PCT Filed: Dec. 29, 2015
(86) PCT No.: PCT/GB2015/054170

§ 371 Date: Jun. 30, 2017

(30) Foreign Application Priority Data
Dec. 31, 2014 (GB) 1423376.1

(51) Int. Cl. C07C 279/00; C07D 217/06; C07C 311/21; C07C 279/22; A61K 45/06; A61K 31/155; A61K 31/495; A61K 31/472; A61K 31/255; A61K 31/166; C07D 295/215; C07C 279/08

(52) U.S. Cl. CPC C07C 279/00; C07D 295/215; C07D 217/06; C07C 311/21; C07C 279/22; C07C 279/08; A61K 31/155; A61K 31/495; A61K 31/472; A61K 31/255; A61K 31/166; A61K 45/06

(57) ABSTRACT

The invention discloses compounds of the formula wherein A, B, X, Y and Z are defined. The compounds of the invention show activity against a range of bacteria, and are useful in the treatment or prophylaxis of a bacterial infection in an animal.

### BACKGROUND

The present invention relates to broad spectrum guanidine antibiotic compounds that are useful in treating bacterial infections.

Infections caused by bacteria are a growing medical concern as many of these bacteria are resistant to various antibiotics. Such microbes include Escherchia coli, Caulobacter crescentus, Pseudomonas aeruginosa, Agrobacterium tumefaciens, Branhamella catarrhalis, Citrobacter diversus, Enterobacter aerogenes, Enterobacter cloacae, Enterobacter sakazakii, Enterobacter asburiae, Pantoea agglomerans, Klebsiella pneumoniae, Klebsiella oxytoca, Klebsiella rhinoscleromatis, Proteus mirabilis, Salmonella typhimurium, Salmonella enteriditis, Serratia marcescens, Shigella sonnei, Neisseria gonorrhoeae, Acinetobacter baumannii, Acinetobacter calcoaceticus, Acinetobacter lwoffi, Salmonella enteriditis, Fusobacterium nucleatum, Veillonella parvula, Bacteroides forsythus, Actinobacillus actinomycetemcomitans, Aggregatibacter actinomycetemcomitans, Porphyromonas gingivalis, Helicobacter pylori, Francisella tularensis, Yersinia pestis, Borrelia burgdorferi, Neisseria meningitidis and Haemophilus influenzae.

Guanidine-containing antibiotics are known, for example, from WO2013/106761 and J. Med. Chem.2012, 55: 10160-10176.

### SUMMARY OF THE INVENTION

In one embodiment, the invention provides compounds of Formula I

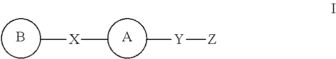

wherein

A is an optionally substituted aryl or heteroaryl group;

B is an optionally substituted aryl or heteroaryl group; and

X is a group —(R¹)W(R²)—,

Y is selected from a chemical bond, C1-C3 straight or branched alkylene, C═O, C(R⁵)═N, and C1-C3 straight or branched oxyalkylene;

W is selected from O, S, SO, SO₂, NH, N(C1-C6)alkyl, C(O)O, C(O)NH, SO₂NH, Si(C1-C6)alkyl₂, and C(R³)═C(R⁴),

Z is a group

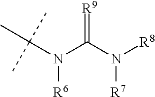

R¹ and R² are independently selected from a chemical bond and C1-C3 straight or branched alkylene,

R³ and R⁴ are independently selected from H and (C1-C6)alkyl

R⁵ is selected from H and (C1-C6)alkyl;

R⁶, R⁷, and R⁸ are independently selected from H, (C1-C6)alkyl, CN, NO₂, (C1-C6)acyl, NH₂, NH(C1-C6)alkyl, N((C1-C6)alkyl)₂, C(═NH)NH₂, and (C1-C6)carboxyalkoxy,

R⁹ is selected from O, S, SO, SO₂, NR¹⁰, and CR¹¹R¹²

R¹⁰ is selected from H, (C1-C6)alkyl, CN, NO₂, (C1-C6)acyl, NH₂, NH(C1-C6)alkyl, N((C1-C6)alkyl)₂, C(═NH)NH₂, and (C1-C6)carboxyalkoxy,

R¹¹ and R¹² are independently selected from H, (C1-C6)alkyl, CN, and NO₂,

or, taken together with the atoms to which they are attached, R⁶ and R⁷, R⁷ and R⁸, R⁸ and R¹⁰, R⁶ and R¹⁰, R⁶ and R¹¹, or R⁸ and R¹¹ form an optionally substituted 3 to 6 membered heteroaryl or heterocyclyl ring optionally containing 1 or 2 further heteroatoms selected from O, N and S;

or, taken together with the atoms to which they are attached, R⁶, R⁷, R⁸, R¹⁰, or R¹¹ form an optionally substituted 3 to 6 membered heteroaryl or heterocyclyl ring fused to ring A,

or a pharmaceutically active salt or N-oxide thereof.

According to a second embodiment, the invention provides a pharmaceutical composition comprising a compound of Formula I as defined above and a pharmaceutically acceptable vehicle.

According to a third embodiment, the invention provides a compound or a composition as defined above for use as a medicament.

According to a fourth embodiment, the invention provides a compound or a composition as defined above for use in the treatment or prophylaxis of a bacterial infection in an animal.

According to a fifth embodiment, the invention provides a compound or a composition as defined above for use in the treatment or prophylaxis of a bacterial infection in an animal.

According to a sixth embodiment, the invention provides a method of treatment or prophylaxis of a bacterial infection in an animal comprising administering to an animal in need thereof a therapeutically effective dose of a compound or a composition as defined above.

### DETAILED DESCRIPTION

Preferably, A is an aryl group selected from phenyl, thiazolyl, pyridyl, imidazolyl and benzothiazole. More preferably, A is a phenyl group, optionally substituted with from one to three groups independently selected from halo, C1-C6 alkyl, C1-C6 haloalkyl, C1-C6 alkoxy, C1-C6 haloalkoxy, nitro, cyano and C1-C6 thioalkyl. Still more preferably, A is a phenyl group optionally substituted with one or two groups independently selected from Cl, F and methoxy.

In a preferred embodiment, A has the formula

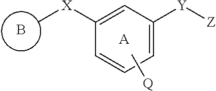

wherein B, X, Y and Z are as herein defined and Q is optionally present and represents from one to three groups independently selected from halo, C1-C6 alkyl, C1-C6 haloalkyl, C1-C6 alkoxy, C1-C6 haloalkoxy, nitro, cyano and C1-C6 thioalkyl. More preferably, A has the formula

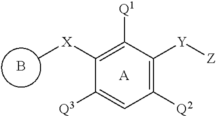

wherein Q¹, Q² and Q³ are independently selected from H, Cl, F, and methoxy. Preferably, Q² and Q³ are H. Preferably, Q¹ is Cl or H. More preferably, Q¹ is H.

In an alternative preferred embodiment, A has the formula

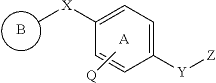

wherein A, B, X, Y and Z are as defined above.

Preferably, B is an aryl group selected from phenyl, thiazolyl, pyridyl, and benzothiazole. More preferably, B is a phenyl group, optionally substituted with from one to three groups independently selected from halo, C1-C6 alkyl, C1-C6 haloalkyl, C1-C6 alkoxy, C1-C6 haloalkoxy, nitro, cyano and C1-C6 thioalkyl. Still more preferably, A is a phenyl group, optionally substituted with from one to three groups independently selected from halo, trifluoromethyl and methoxy.

In certain preferred embodiments, B is a group

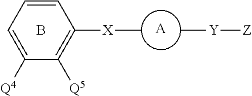

wherein Q⁴ and Q⁵ are independently selected from Cl, methoxy and trifluoromethyl.

In certain preferred embodiments, R¹ is selected from a chemical bond and methylene.

In certain preferred embodiments, R² is selected from a chemical bond and methylene.

In certain preferred embodiments, W is selected from O, C(═O)NH, SO₂NH and CH₂. More preferably, W is O.

In certain very preferred embodiments, X is a group —CH₂O—. In more preferred embodiments, the compounds of the invention have the formula

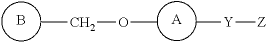

In certain preferred embodiments, Y is selected from a chemical bond, methylene, C(R⁵)═N, C═O and —CH₂O—. More preferably, Y is CH═N.

Preferably, Z is a group of the formula

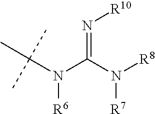

Preferably, at least one of R⁶, R⁷, R⁸ and R¹⁰ is H. More preferably, all of R⁶, R⁷, R⁸ and R¹⁰ are H.

In certain preferred embodiments, taken together with the atoms to which they are attached, R⁶, R⁷, R⁸, R¹⁰, or R¹¹ form an optionally substituted 3 to 6 membered heteroaryl or heterocyclyl ring fused to ring A. Preferred rings are 5 or 6 membered, with 6 membered rings being most preferred. It is preferred that the ring contains a single nitrogen heteroatom. Most preferred is 1,2,3,6-tetrahydropyridine.

In certain preferred embodiments, the compound has the formula

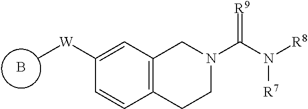

wherein B, W, R⁷, R⁸ and R⁹ are as defined above.

In certain alternative preferred embodiments, the compound has the formula

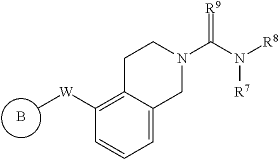

wherein B, W, R⁷, R⁸ and R⁹ are as defined above.

Specifically preferred compounds of the invention are 1-[(E)-[3-[(2,3-dichlorophenyl)methoxy]phenyl]methyleneamino]guanidine; 1-[(E)-[3-[[2-chloro-3-(trifluoromethyl)phenyl]methoxy]phenyl]methyleneamino]guanidine; 7-[(2,3-dichlorophenyl)methoxy]-3,4-dihydro-1H-isoquinoline-2-carboxamidine; 5-[(2,3-dichlorophenyl)methoxy]-3,4-dihydro-1H-isoquinoline-2-carboxamidine; 1-[[3-[(2,3-dichlorophenyl)methoxy]phenyl]methyl]-1-methyl-guanidine; 1-[[3-[(2,3-dichlorophenyl)methoxy]phenyl]methyl]-1-(methoxyethyl)guanidine; 1-[[3-[(2,3-dichlorophenyl)methoxy]phenyl]methyl]-2-methyl-guanidine; 1-[(E)-[2-chloro-3-[(2-chloro-3-methoxy-phenyl)methoxy]phenyl]methyleneamino]guanidine; 1-[(E)-(3-benzyloxy-2-chloro-phenyl)methyleneamino]guanidine; 1-[(E)-[2-chloro-3-[(4-chlorophenyl)methoxy]phenyl]methyleneamino]guanidine; 1-[(E)-[2-chloro-3-[(2,3-dichlorophenyl)methoxy]phenyl]methyleneamino]guanidine; 1-[(E)-[2-chloro-3-[[2-chloro-3-(trifluoromethyl)phenyl]methoxy]phenyl]methyleneamino]guanidine; 1-[(E)-[2-chloro-3-[(2,4-dichlorophenyl)methoxy]phenyl]methyleneamino]guanidine; 1-[(E)-[2-chloro-3-[(2,5-dichlorophenyl)methoxy]phenyl]methyleneamino]guanidine; 1-[(E)-[2-chloro-3-[(3,4-dichlorophenyl)methoxy]phenyl]methyleneamino]guanidine; 1-[(E)-[2-chloro-3-[(2,3,5-trichlorophenyl)methoxy]phenyl]methyleneamino]guanidine; 1-[(E)-[2-chloro-3-[[3-(trifluoromethyl)phenyl]methoxy]phenyl]methyleneamino]guanidine; 1-[(E)-[2-chloro-3-[[4-(trifluoromethyl)phenyl]methoxy]phenyl]methyleneamino]guanidine; N-[(4-guanidinophenyl) methyl]benzamide; 1-[4-(benzenesulfonamidomethyl)phenyl]guanidine; N-[3-(guanidinomethyl)phenyl]benzamide; N-[4-(guanidinomethyl)phenyl]benzamide; 1-[[4-[(3-chlorophenyl)sulfonylamino]phenyl]methyl]guanidine; 1-[[4-[(4-chlorophenyl)sulfonylamino]phenyl]methyl]guanidine; 1-[[4-[(2,3-dichlorophenyl)sulfonylamino]phenyl]methyl]guanidine; 1-[[4-[[3-(trifluoromethyl)phenyl]sulfonylamino]phenyl]methyl]guanidine; 1-[[3-[(3-chlorophenyl)sulfonylamino]phenyl]methyl]guanidine; 1-[[3-[(4-chlorophenyl)sulfonylamino]phenyl]methyl]guanidine; 1-[[3-[(2,3-dichlorophenyl)sulfonylamino]phenyl]methyl]guanidine; 1-[[3-[[3-(trifluoromethyl)phenyl]sulfonylamino]phenyl]methyl]guanidine; 1-[[3-[[2-chloro-3-(trifluoromethyl)phenyl]methoxy]phenyl]methyl]guanidine; 1-[[3-[(2,3-dichlorophenyl) methoxy]phenyl]methyl]guanidine; 1-[[4-[(2,3-dichlorophenyl)methoxy]phenyl]methyl]guanidine; 1-[[3-[(2,3-dichlorophenyl)methoxy]-2,6-difluoro-phenyl]methyl]guanidine; 1-[[3-[[3-(trifluoromethyl)phenyl]methoxy]phenyl]methyl]guanidine; 1-[[2,6-difluoro-3-[[3-(trifluoromethyl)phenyl]methoxy]phenyl]methyl]guanidine; 1-[[3-[(2,5-dichlorophenyl)methoxy]phenyl]methyl]guanidine; 1-[[3-[[4-(trifluoromethyl)phenyl]methoxy]phenyl]methyl]guanidine; 1-[[3-[(3,4-dichlorophenyl)methoxy]phenyl]methyl]guanidine; 1-[[3-[(4-chlorophenyl)methoxy]phenyl]methyl]guanidine; 1-[[3-[(4-bromophenyl) methoxy]phenyl]methyl]guanidine; 1-[[4-[(3,4-dichlorophenyl)methoxy]phenyl]methyl]guanidine; 1-[[3-[(3-chlorophenyl)methoxy]phenyl]methyl]guanidine; 3-benzyloxy-N-carbamimidoyl-benzamide; 1-[(3-benzyloxyphenyl)methyl]guanidine; 1-[[3-[(2,4-dichlorophenyl)methoxy]phenyl]methyl]guanidine; 1-[[3-[(4-fluorophenyl)methoxy]phenyl]methyl]guanidine; 1-[(4-benzyloxyphenyl)methyl]guanidine; 1-(4-benzyloxyphenyl)guanidine; 1-[[4-[(3-chlorophenyl)methoxy]phenyl]methyl]guanidine; 1-[4-[(3-chlorophenyl)methoxy]phenyl]guanidine; 1-[[3-[(4-chlorophenyl)methoxy]-4-methoxy-phenyl]methyl]guanidine; 1-[[4-[(4-chlorophenyl)methoxy]phenyl]methyl]guanidine; 7-(4-tert-butylphenyl)-3,4-dihydro-1H-isoquinoline-2-carboxamidine; 1-[[3-(4-tert-butylphenyl)phenyl]methoxy]guanidine; 1-[4-[(4-chlorophenyl)methoxy]phenyl]guanidine; 1-[(3-phenylphenyl)methoxy]guanidine; 1-[[3-[(3-chlorophenyl)methoxy]-4-methoxy-phenyl]methyl]guanidine; 1-[(3-phenoxyphenyl)methoxy]guanidine; 1-[(E)-[3-[(4-chlorophenyl)methoxy]phenyl]methyleneamino]guanidine; 1-[(E)-[3-[(4-trifluoromethylphenyl)methoxy]phenyl]methyleneamino]guanidine; 1-[[3-(phenyl)phenyl]methoxy]guanidine; (E)-Amino(2-(3-((4-chlorobenzyl)oxy)benzylidene)hydrazinyl)methanimine; (E)-Amino(2-(3-((4-(trifluoromethyl)benzyl)oxy)benzylidene) hydrazinyl) methanimine; (E)-Amino(2-(3-((3-chlorobenzyl)oxy)benzylidene)hydrazinyl)methanimine; (E)-Amino(2-(3-((4-(trifluoromethyl)benzyl) oxy)benzylidene)hydrazinyl) methanimine; (E)-2-(3-((3,4-dichlorobenzyl)oxy)-4-methoxybenzylidene) hydrazine-1-carboximidamide; (E)-Amino(2-(2-chloro-3-((3-chlorobenzyl)oxy)benzylidene)hydrazinyl) methanimine; 1-(4-Chlorobenzyl)-1H-imidazole-2-carbaldehyde; 1-(4-(Trifluoromethyl)benzyl)-1H-imidazole-2-carbaldehyde; 1-(3-Chlorobenzyl)-1H-imidazole-2-carbaldehyde; 1-(3-(Trifluoromethyl)benzyl)-1H-imidazole-2-carbaldehyde; 1-(3-Chlorobenzyl)-1H-pyrrole-2-carbaldehyde; 1-(4-Chlorobenzyl)-1H-pyrrole-2-carbaldehyde; 1-(3-(Trifluoromethyl)benzyl)-1H-pyrrole-2-carbaldehyde; and 1-(4-(Trifluoromethyl)benzyl)-1H-pyrrole-2-carbaldehyde, together with pharmaceutically acceptable salts and esters thereof.

A separate aspect of the invention relates to compounds of the formula

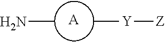

wherein A, Y and Z have the values ascribed above. Compounds of this type are useful intermediates in the synthesis of compounds of formula 1. Moreover, some compounds have intrinsic antibiotic activity in their own right, and are useful in the methods and compositions described elsewhere herein. Specific examples are 1-[(3-aminophenyl)methyl]guanidine and 1-[(4-aminophenyl)methyl]guanidine.

### Definitions

As used herein the term “aryl” refers to aromatic monocyclic or multicyclic groups containing from 5 to 15 carbon atoms. Aryl groups include, but are not limited to groups such as unsubstituted or substituted fluorenyl, unsubstituted or substituted phenyl, and unsubstituted or substituted naphthyl. When referring to said aryl being substituted, said substitution may be at any position on the ring, other than the point of attachment to the other ring system of a compound of the invention. Therefore, any hydrogen atom on the aryl ring may be substituted with a substituent defined by the invention. In embodiments where the aryl is a phenyl ring, said substitution may be at the meta- and/or ortho- and/or para-position relative to the point of attachment.

As used herein the term “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

The term “heterocycle” or “heterocyclyl”, as used herein, refers to a monocyclic, or bicyclic, group, unless otherwise specified, containing 1 to 4 heteroatoms selected from N, O, S, SO, SO₂ NH or N(C1-C6)alkyl. Heterocyclic groups optionally contain 1 or 2 double bonds. Heterocyclic groups include, but are not limited to, azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydro-thiadiazinyl, morpholinyl, oxetanyl, tetrahydrodiazinyl, oxazinyl, oxathiazinyl, indolinyl, isoindolinyl, quinuclidinyl, chromanyl, isochromanyl, and benzoxazinyl.

As used herein, the term “alkylene” refers to a divalent, saturated group consisting of carbon and hydrogen atoms. Examples of alkylene groups include methylene, ethylene, propylene, n-butylene, and the like. The alkylene is attached to the rest of the molecule through a single bond and to the radical group through a single bond. The points of attachment of the alkylene to the rest of the molecule and to the radical group can be through one carbon or any two carbons within the carbon chain.

As used herein, “oxyalkylene” refers to a bivalent radical comprising an alkylene group as defined above that is substituted with an oxy group, such as, for example, oxymethylene, and oxydimethylene.

As used herein, the term “alkyl,” unless otherwise specified, refers to a saturated straight or branched hydrocarbon chain of typically C1 to C6, and specifically includes methyl, ethyl, propyl, isopropyl, butyl, isobutyl, t-butyl, pentyl, isopentyl, neopentyl, hexyl, isohexyl, cyclohexyl, cyclohexylmethyl, 3-methylpentyl, 2,2-dimethylbutyl, and 2,3-dimethylbutyl, and the like.

The term “alkoxy,” as used herein, refers to an alkyl group, as defined herein, appended to the parent molecular moiety through an oxygen atom. Examples include methoxy, ethoxy, propoxy, isopropoxy, butoxy, isobutoxy, t-butoxy, pentoxy, isopentoxy, neopentoxy, hexyloxy, isohexyloxy, cyclohexyloxy, 2,2-dimethylbutoxy, and 2,3-dimethylbutoxy, and the like.

As used herein, the term “haloalkyl” refers to an alkyl as defined herein, which is substituted by one or more halo groups as defined herein. The haloalkyl can be monohaloalkyl, dihaloalkyl, trihaloalkyl, or polyhaloalkyl including perhaloalkyl. A monohaloalkyl can have one iodo, bromo, chloro or fluoro within the alkyl group. Chloro and fluoro are preferred. Dihaloalkyl and polyhaloalkyl groups can have two or more of the same halo atoms or a combination of different halo groups within the alkyl. Examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms, e.g, trifluoromethyl.

The term “haloalkoxy,” as used herein, refers to a haloalkyl group, as defined herein, appended to the parent molecular moiety through an oxygen atom. Examples include fluoromethoxy, difluoromethoxy, trifluoromethoxy, chloromethoxy, dichloromethoxy, trichloromethoxy, pentafluoroethoxy, heptafluoropropoxy, difluorochloromethoxy, dichlorofluoromethoxy, difluoroethoxy, difluoropropoxy, dichloroethoxy and dichloropropoxy and the like.

The term “acyl” as used herein means an organic radical having 1 to 6 carbon atoms (branched or straight chain) derived from an organic acid by removal of the hydroxyl group. Examples of acyl groups are methanoyl, ethanoyl, propanoyl, n-butanoyl, pivaloyl, and the like.

As used herein, the term “alkoxyalkyl” refers to an alkyl group, as defined above, substituted with an alkoxy group as defined above. Examples are methoxymethylene, methoxy ethylene, ethoxymethylene and ethoxyethylene, and the like.

Where a group is described as “optionally substituted”, for example in the case of aryl, heteroaryl, and heterocyclyl, the substituents are, unless otherwise specified, selected from the group consisting of C1-C3alkyl, C1-C3alkoxy, halogen, C1-C3haloalkyl, C1-C3haloalkoxy, nitro, cyano, and hydroxyl.

As salts in the context of the invention, the pharmaceutically acceptable salts of the compounds of the invention are preferred.

Pharmaceutically acceptable salts of the compounds of the invention may be acid addition salts of the substances according to the invention with mineral acids, carboxylic acids or sulphonic acids. Especially preferred are salts with hydrochloric acid, hydrobromic acid, sulfuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, citric acid, fumaric acid, maleic acid or benzoic acid.

However, certain compounds of the invention can include salts with customary bases, such as alkali metal salts (eg sodium or potassium salts), alkaline earth metal (eg calcium or magnesium salts) or ammonium salts derived from ammonia or organic amines.

Certain of the compounds of the invention may exist in stereoisomeric forms, either as image and mirror image (enantiomers), or which do not behave as image and mirror image (diastereomers). The invention relates to enantiomers, diastereomers and their respective mixtures, as well as racemic forms.

The invention also includes prodrugs of the compounds of the invention. “Prodrugs” are in the present invention refer to those derivatives of the compounds of general formula (I), which are themselves biologically less active, or inert, but are converted under physiological conditions in the corresponding biologically active form (for example by metabolism, solvolysis, or any other way).

Therapeutic Uses

In therapeutic use, the compounds of this invention may be administered in the form of conventional pharmaceutical composition appropriate for the intended use as antibacterials. Such compositions may be formulated so as to be suitable for oral, parenteral or topical administration. The active ingredient may be combined in admixture with nontoxic pharmaceutical carrier may take a variety of forms, depending on the form of preparation desired for administration, i.e. oral, parenteral, or topical. When the compounds are employed as antibacterials, they can be combined with one or more pharmaceutically acceptable carriers, for example, solvents, diluents and the like, and may be administered orally in such forms as tablets, capsules, dispersible powders, granules, or suspensions containing, for example, from about 0.05 to 5% of suspending agent, syrups containing, for example, from about 10 to 50% of sugar, and elixirs containing for example, from about 20 to 50% ethanol and the like, or parenterally in the form of sterile injectable solutions or suspensions containing from about 0.05 to 5% suspending agent in an isotonic medium. Such pharmaceutical preparations may contain, for example, from about 25 to about 90% of the active ingredient in combination with the carrier, more usually between about 5% and 60% by weight.

An effective amount of compound from 0.001 mg/kg of body weight to 100.0 mg/kg of body weight should be administered one to five times per day via any typical route of administration including but not limited to oral, parenteral (including subcutaneous, intravenous, intramuscular, intrasternal injection or infusion techniques), topical or rectal, in dosage unit formulations containing conventional non-toxic pharmaceutically acceptable carriers, adjuvants and vehicles. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition of the host undergoing therapy.

Additionally, the antibacterially effective amount of the antibiotics of the invention may be administered at a dosage and frequency without inducing side effects commonly experienced with conventional antibiotic therapy which could include hypersensitivity, neuromuscular blockade, vertigo, photosensitivity, discoloration of teeth, hematologic changes, gastrointestinal disturbances, ototoxicity, and renal, hepatic, or cardiac impairment. Further the frequency and duration of dosage may be monitored to substantially limit harmful effects to normal tissues caused by administration at or above the antibacterially effective amount of the antibiotics of the invention.

These active compounds may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA. These active compounds may also be administered parenterally or intraperitoneally. Solutions or suspensions of these active compounds as a free base or pharmacologically acceptable salt can be prepared in glycerol, liquid, polyethylene glycols and mixtures thereof in oils. Under ordinary conditions of storage and use, these preparations contain a preservative. The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oil.

The invention accordingly provides a pharmaceutical composition which comprises a compound of this invention in combination or association with a pharmaceutically acceptable carrier. In particular, the present invention provides a pharmaceutical composition which comprises an antibacterially effective amount of a compound of this invention and a pharmaceutically acceptable carrier.

The present invention further provides a method of treating bacterial infections in animals including man, which comprises administering to the afflicted animals an antibacterially effective amount of a compound or a pharmaceutical composition of a compound of the invention.

The compounds of the invention are useful to treat bacterial infections including infections caused by Gram-negative bacterial strains, Gram-positive bacterial strains and multiple drug-resistant bacterial strains

Gram-negative bacterial strains include Escherchia coli, Caulobacter crescentus, Pseudomonas aeruginosa, Agrobacterium tumefaciens, Branhamella catarrhalis, Citrobacter diversus, Enterobacter aerogenes, Enterobacter cloacae, Enterobacter sakazakii, Enterobacter asburiae, Pantoea agglomerans, Klebsiella pneumoniae, Klebsiella oxytoca, Klebsiella rhinoscleromatis, Proteus mirabilis, Salmonella typhimurium, Salmonella enteriditis, Serratia marcescens, Shigella sonnei, Neisseria gonorrhoeae, Acinetobacter baumannii, Acinetobacter calcoaceticus, Acinetobacter lwoffi, Fusobacterium nucleatum, Veillonella parvula, Bacteroides forsythus, Actinobacillus actinomycetemcomitans, Aggregatibacter actinomycetemcomitans, Porphyromonas gingivalis, Helicobacter pylori, Francisella tularensis, Yersinia pestis, Borrelia burgdorferi, Neisseria meningitidis and Haemophilus influenza.

Gram-positive bacterial strains include Staphylococcus aureus, Staphylococcus epidermidis, Staphylococcus saprophyticus, Streptococcus pyogenes, Streptococcus faecalis, Enterococcus faecalis, Enterococcus faecium, Bacillus subtilis, Bacillus anthracis, Bacillus cereus, Micrococcus luteus, Mycobacterium tuberculosis, Clostridium difficile, Propionibacterium acnes, Streptococcus mutans, Actinomyces viscosus, Actinomyces naeslundii, Streptococcus sanguis, Streptococcus pneumoniae, Streptococcus viridans and Streptococcus salivarius.

Multiple drug-resistant bacterial strains include methicillin-resistant Staphylococcus aureus, vancomycin-resistant Enterococci, multiple drug-resistant Mycobacterium tuberculosis, and multidrug-resistant Clostridium difficile.

In one embodiment compounds of the present invention may be administered as a composition used to treat and/or prevent a bacterial infection wherein the bacterial cell uses polymerized FtsZ protein, or a homolog thereof, to facilitate cytokinesis. To this end, compounds of the present invention may be administered to treat staph infections, tuberculosis, urinary tract infections, meningitis, enteric infections, wound infections, acne, encephalitis, skin ulcers, bed sores, gastric and duodenal ulcers, eczema, periodontal disease, gingivitis, halitosis, anthrax, tularemia, endocarditis, prostatitis, osteomyelitis, Lyme disease, pneumonia, or the like.

In another embodiment of the invention, the compound of the invention may be used as a combined preparation for simultaneous, separate or sequential administration together with one or more further antibiotics selected from macrolide antibiotics, β-lactam antibiotics, tetracycline antibiotics, and quinolone antibiotics. Preferred antibiotics for use in such combined preparations are azithromycin, clarithromycin, dirithromycin, erythromycin, roxithromycin, telithromycin, Carbomycin A, josamycin, kitasamycin, midecamicine, oleandomycin, spiramycin, tylosin, troleandomycin, aztreonam, imipenem, meropenem, ertapenem, doripenem, panipenem/betamipron, biapenem, PZ-601, cefixime, cefdinir, cefditoren, cefoperazone, cefotaxime, cefpodoxime, ceftazidime, ceftibuten, ceftizoxime, ceftriaxone, cefepime, demeclocycline, doxycycline, minocycline, oxytetracycline, tetracycline, ciprofloxacin, enoxacin, gatifloxacin, levofloxacin, lomefloxacin, moxifloxacin, norfloxacin, ofloxacin, and trovafloxacin, preferably ceftazidime, imipenem/cilastatin, meropenem, aztreonam, oxytetracycline, azithromycin, clarithromycin, dirithromycin, erythromycin, roxithromycin, spiramycin and ciprofloxacin. The following examples illustrate the preparation of the compounds of the invention by synthetic procedures and as such are not to be considered as limiting the invention set forth in the claims appended hereto.

### Chemical Synthesis of Benzyl Guanidine and Phenyl Guanidine Derivatives

The target compounds 4a-4p were synthesized from the corresponding 3-aminomethylphenol derivatives (1a-1c) via a guanylation reaction on the benzyl amine group, followed by the benzylation of the phenol group under a basic condition, and finally the N,N′-di-Boc deprotection in the presence of trifluoroacetic acid. All of the final compounds (4a-4p) were obtained as guanidium trifluoroacete salts as shown in scheme 1. Compound 4g and 4l were also converted to their corresponding hydrogen chloride salt (4g.HCl and 4l.HCl).

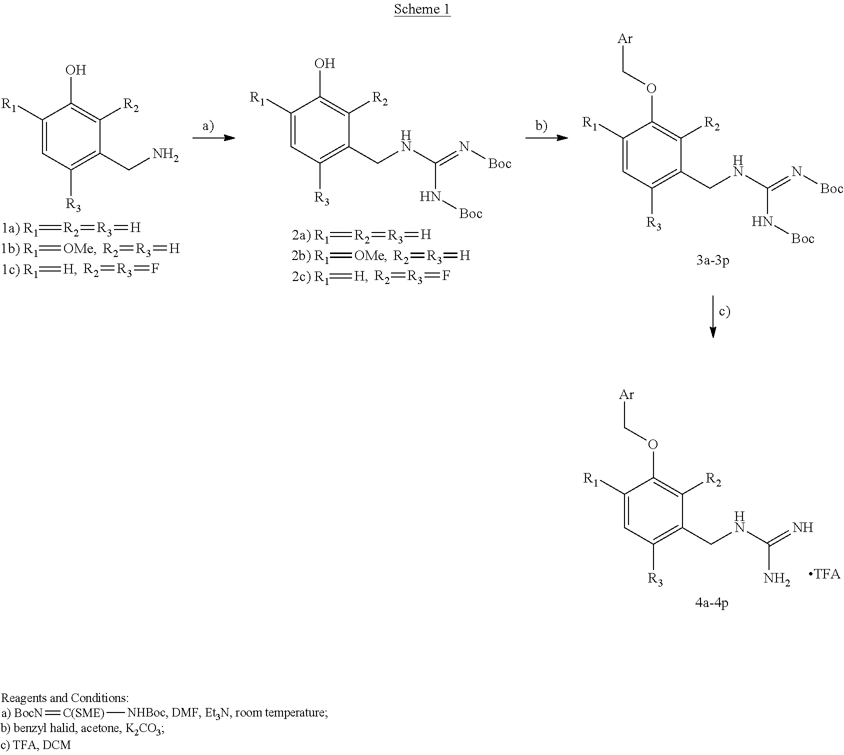

TABLE 1

| Cpd | Ar | R₁ | R₂ | R₃ |
|---|---|---|---|---|
| 3a | Ph | H | H | H |
| 3b | 4-Cl—Ph | H | H | H |
| 3c | 3-Cl—Ph | H | H | H |
| 3d | 2,4-di-Cl—Ph | H | H | H |
| 3e | 3,4-di-Cl—Ph | H | H | H |
| 3f | 2,5-di-Cl—Ph | H | H | H |
| 3g | 2,3-di-Cl—Ph | H | H | H |
| 3h | 4-CF₃—Ph | H | H | H |
| 3i | 3-CF₃—Ph | H | H | H |
| 3j | 4-Br—Ph | H | H | H |
| 3k | 4-F—Ph | H | H | H |
| 3l | 2-Cl-3-CF₃—Ph | H | H | H |
| 3m | 3-Cl—Ph—Ph | OMe | H | H |
| 3n | 4-Cl—Ph | OMe | H | H |
| 3o | 2,3-di-Cl—Ph | H | F | F |
| 3p | 3-CF₃—Ph | H | F | F |
| 4a | Ph | H | H | H |
| 4b | 4-Cl—Ph | H | H | H |
| 4c | 3-Cl—Ph | H | H | H |
| 4d | 2,4-di-Cl—Ph | H | H | H |
| 4e | 3,4-di-Cl—Ph | H | H | H |
| 4f | 2,5-di-Cl—Ph | H | H | H |
| 4g | 2,3-di-Cl—Ph | H | H | H |
| 4g.HCl | 2,3-di-Cl—Ph | H | H | H |
| 4h | 4-CF₃—Ph | H | H | H |
| 4i | 3-CF₃—Ph | H | H | H |
| 4j | 4-Br—Ph | H | H | H |
| 4k | 4-F—Ph | H | H | H |
| 4l | 2-Cl-3-CF₃—Ph | H | H | H |
| 4l.HCl | 2-Cl-3-CF₃—Ph | H | H | H |
| 4m | 3-Cl—Ph | OMe | H | H |
| 4n | 4-Cl—Ph | OMe | H | H |
| 4o | 2,3-di-Cl—Ph | H | F | F |
| 4p | 3-CF₃—Ph | H | F | F |

The reductive amination reaction of the aldehyde 5 generated the amino intermediates 7a or 7 b, which underwent a guanylation reaction to form the N,N′-di-Boc protected guanidine derivatives (8a or 8b). Deprotection of the Boc groups under the condition of trifluoroacetic acid generated the guanidium trifluoroacetate salt (9a-9b). Compound 9a was subsequently converted to the hydrogen chloride salt (10a) with a HCl-methanol solution (scheme 2).

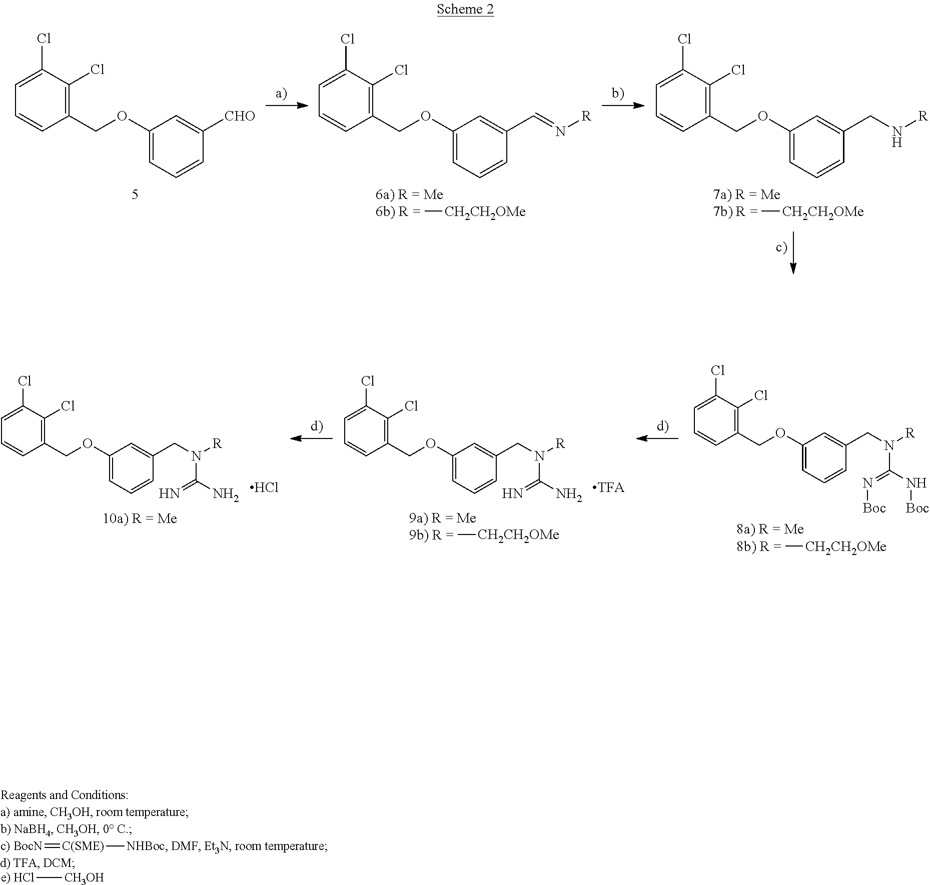

The key intermediate 13 was obtained through the reduction of the aldehyde 5, followed by the halogenation of the alcohol 11, and finally the alkylation of S-methyl-N,N′-bis(tert-butoxycarbonyl)isothiourea under a basic condition. The compound 13 was then subjected the nucleophilic substitution with methylamine to afford the N,N′-di-Boc protected guanidine 14, which was then hydrolysed in TFA to give the final compound 15 (scheme 3).

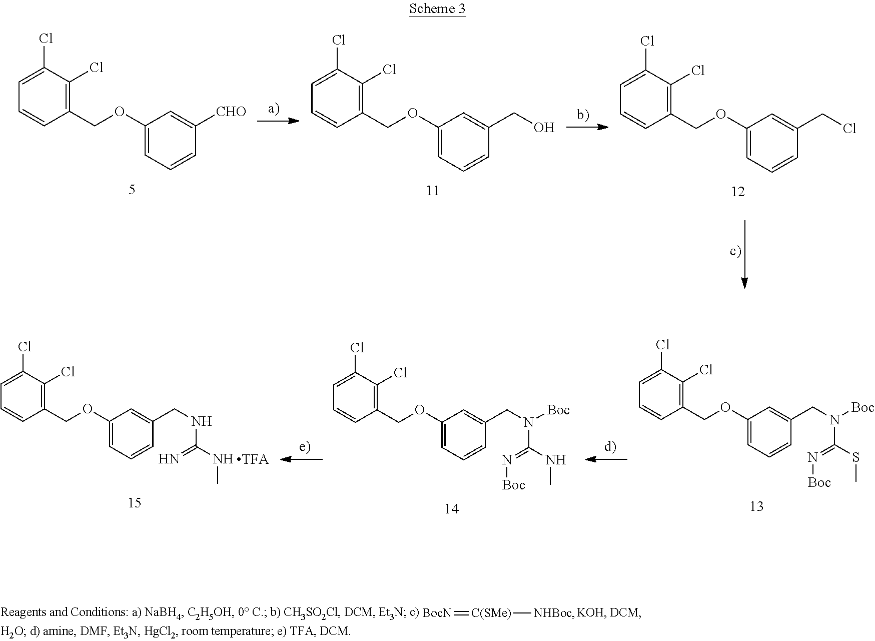

The benzyl guanidine derivatives (19a-19e) with the para-benzyloxy substituent were prepared in the similar way as that of the meta-substituted 4a-4p. All of the target compounds were obtained as the TFA salt (scheme 4).

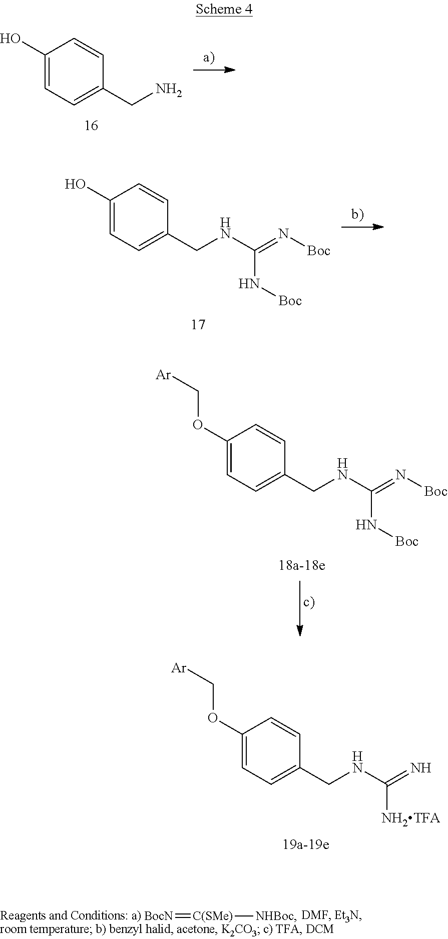

TABLE 2

| Compound | Ar |
|---|---|
| 18a | Ph |
| 18b | 4-Cl—Ph |
| 18c | 3-Cl—Ph |
| 18d | 3,4-di-Cl—Ph |
| 18e | 2,3-di-Cl—Ph |
| 19a | Ph |
| 19b | 4-Cl—Ph |
| 19c | 3-Cl—Ph |
| 19d | 3,4-di-Cl—Ph |
| 19e | 2,3-di-Cl—Ph |

Guanylation reaction of para-aminophenol generated the key intermediate 21, which was subsequently subjected to the benzylation reaction to afford the N,N′-di-Boc protected guanidine derivatives (22a-22c). Deprotection of the Boc groups of compounds 22a-22c using TFA gave the target compounds 23a-23c as shown in scheme 5. Compound 23d was prepared using the same method (scheme 5).

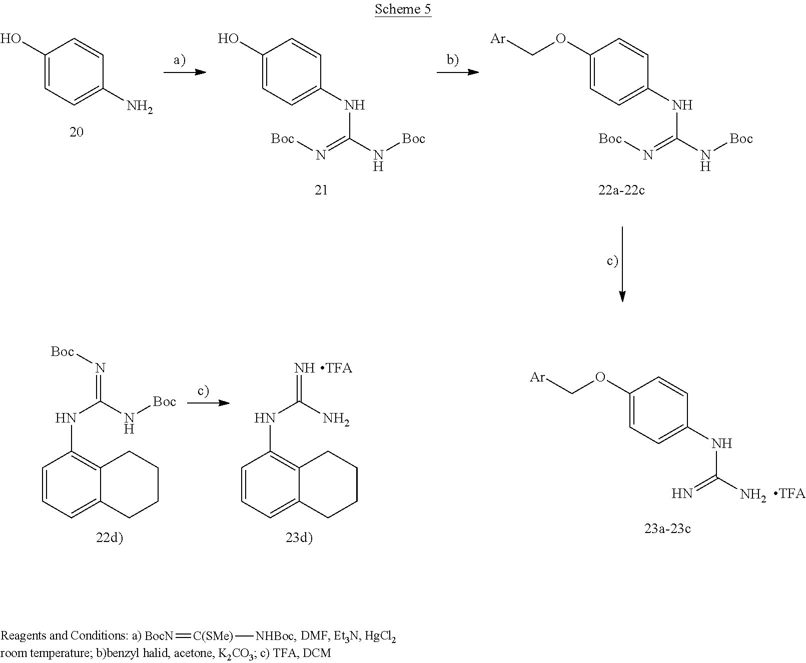

TABLE 3

| Cpd | Ar |
|---|---|
| 22a | Ph |
| 22b | 4-Cl—Ph |
| 22c | 3-Cl—Ph |
| 23a | Ph |
| 23b | 4-Cl—Ph |
| 23c | 3-Cl—Ph |

The 1,2,3,4-tetrahydroisoquinoline-2-carboximidamide derivatives 26 and 28 were prepared via the route shown in scheme 6. Guanylation of 7-bromo-1,2,3,4-tetrahydroisoquinoline 24 gave the corresponding 2-carboximidamide derivative 25, which was subjected to the Boc deprotection to afford 26. Compound 25 was converted to 28 through a route involving a palladium-catalyzed coupling reaction, followed by the deprotection of Boc groups in TFA (scheme 6). Similarly, compound 33 and 38 were prepared from the corresponding hydroxy substituted 1,2,3,4-tetrahydroisoquinoline by N-Boc protection, benzylation, guanylation and finally deprotection as shown in scheme 7 and scheme 8.

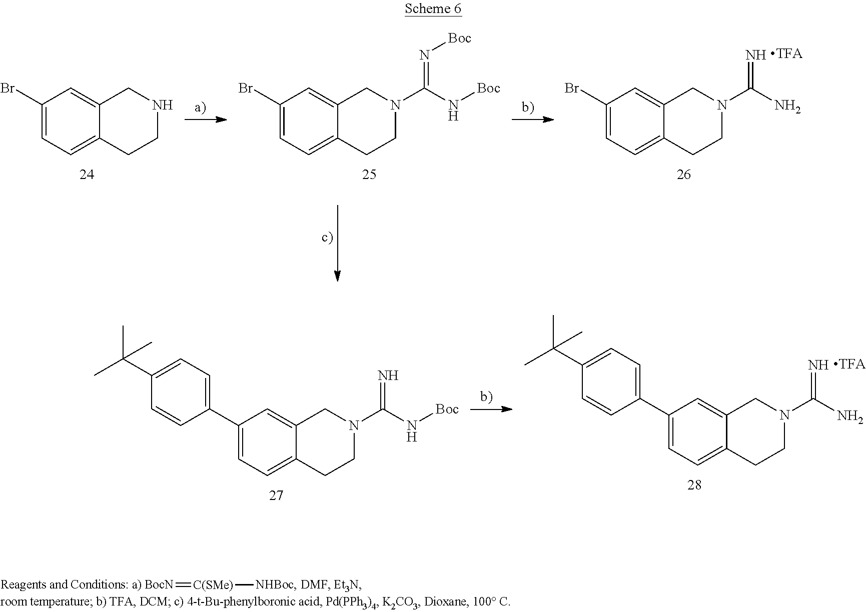

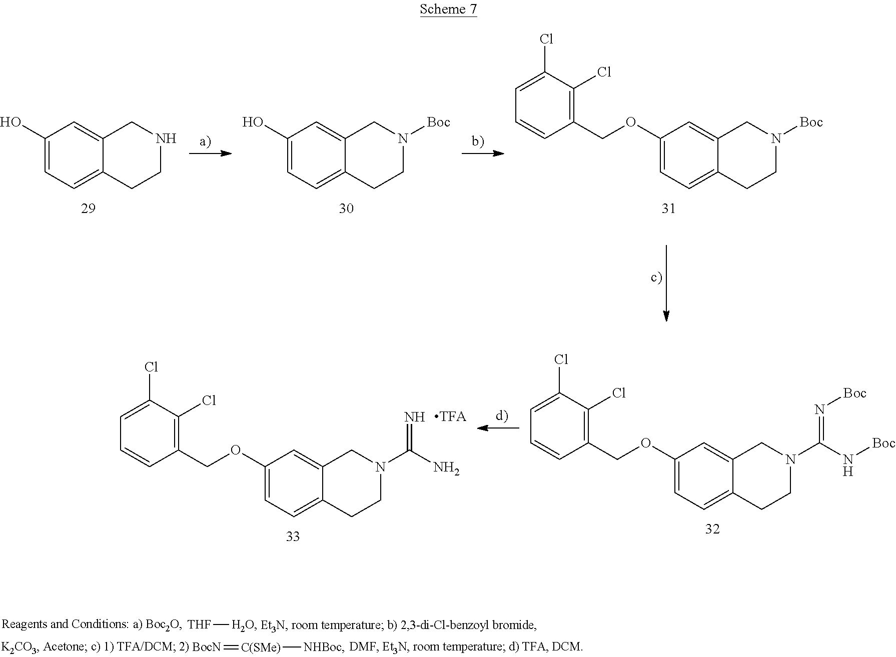

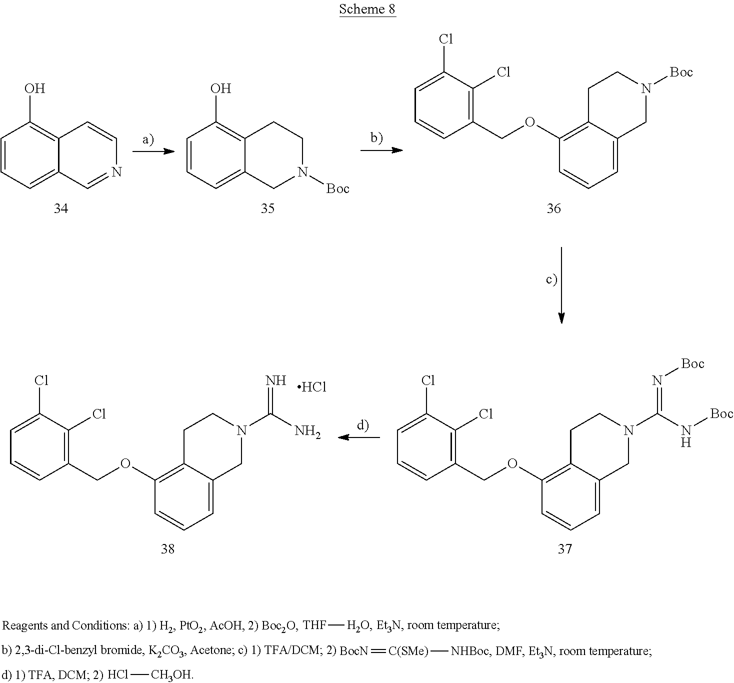

Compound 41a-41c and 44a-44b were all synthesized from the corresponding amine via a guanylation reaction, followed by the removal of the Boc protection groups in the presence of TFA (Scheme 9 and 10). Reacting 3-(benzyloxy)benzoyl chloride 45 with N,N′-di-Boc guanidine produced the intermediate 46, which was treated with TFA to give the target compound 47 (Scheme 11).

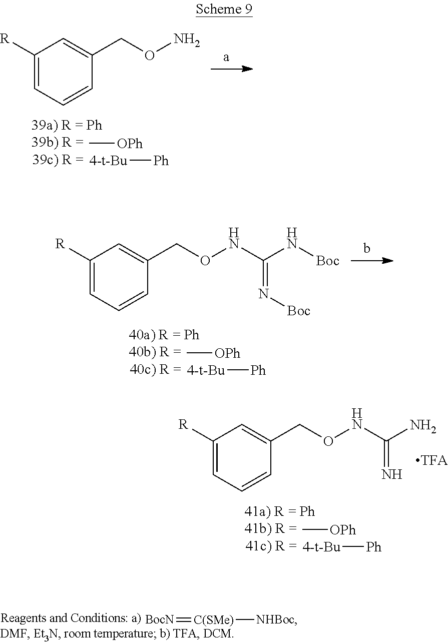

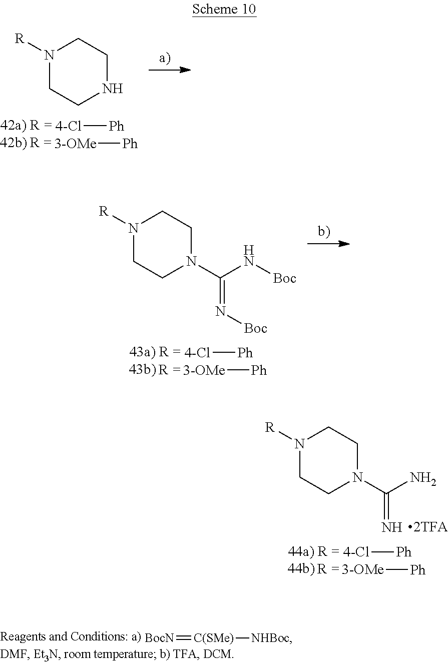

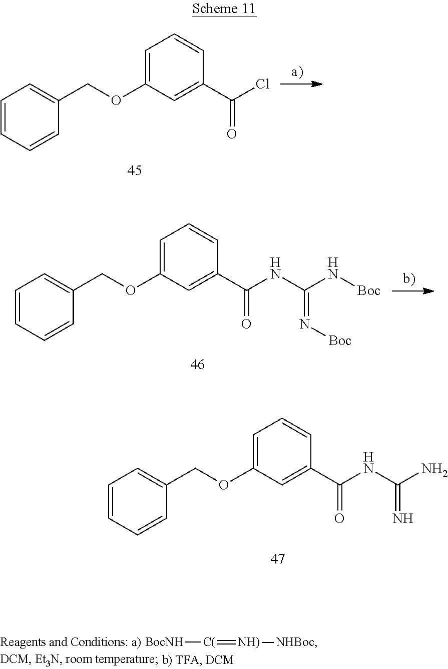

The aminoguanidine derivatives 49a and 49b were prepared by the condensation of corresponding aldehyde with aminoguanidine. The target compounds were obtained in acetate form. Alternatively, compounds 49c-49g was synthesized under the condition of HCl-methanol to give the corresponding hydrogen chloride salt (scheme 12). Similarly, compound 51a-51m was obtained as either a hydrochloride or acetate (scheme 13).

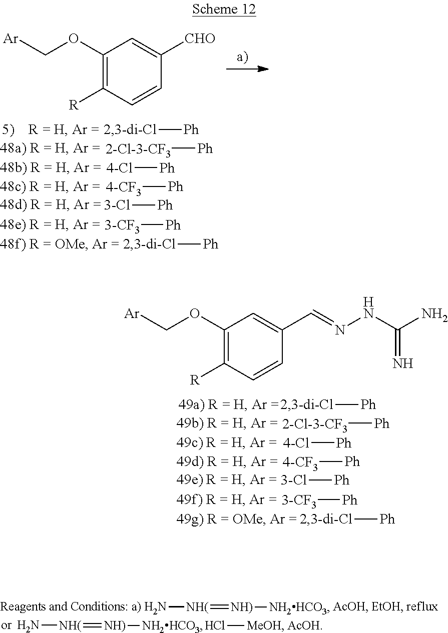

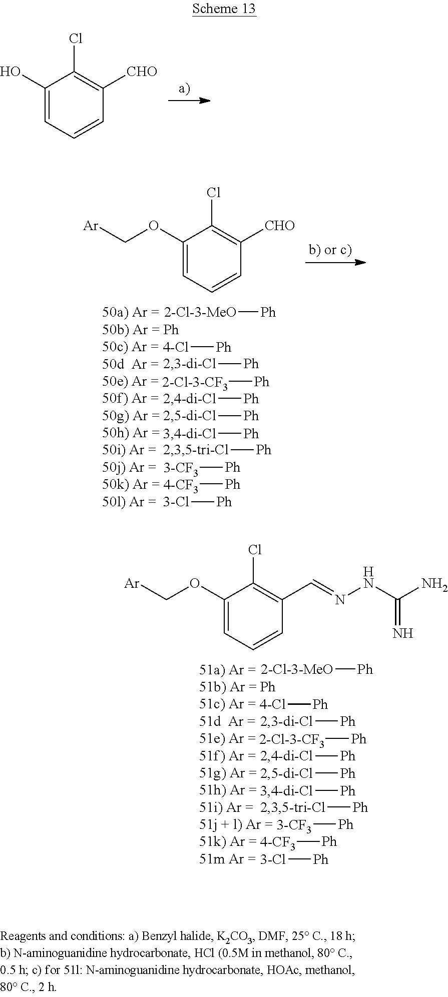

The guanidine derivatives 52a and 52b were prepared by a selective guanylation reaction of the corresponding aminomethylaniline. Compound 52a or 52b was subsequently converted to compounds 55a-55h and 57a-57b via coupling reaction and Boc-deprotection (scheme 14 and 15). Compounds 59a-59b were prepared from 4-aminomethylaniline through a route involving a coupling reaction, followed by guanylation and Boc-deprotection (scheme 16). Benzylation reaction of either 1H-imidazole-2-carbaldehyde or 1H-pyrrole-2-carbaldehyde generated intermediates 60a-60h, which was subsequently condensed with aminoguanidine hydrocarbonate to afford 61a-61h (scheme 17).

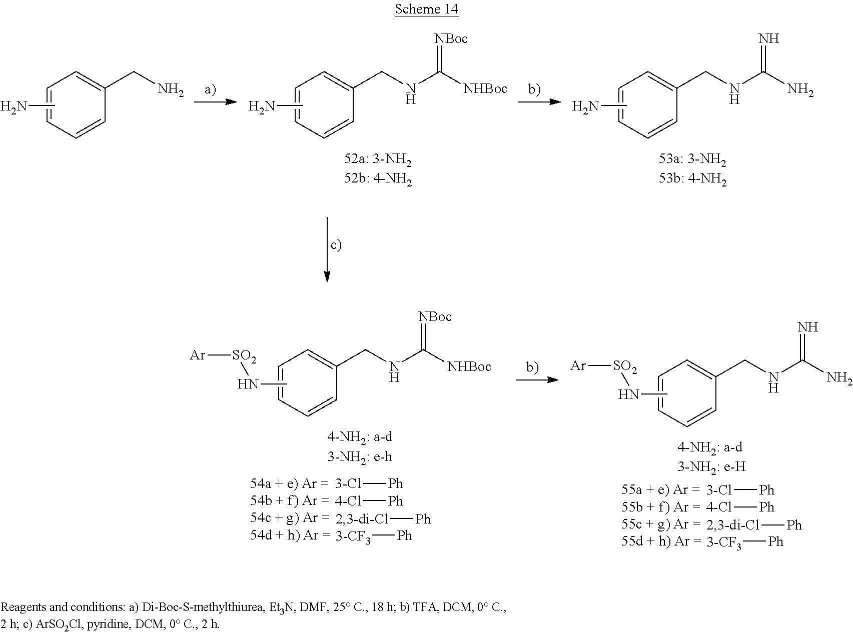

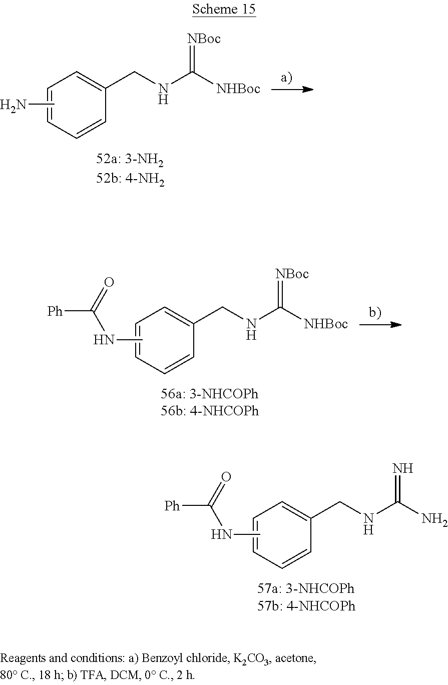

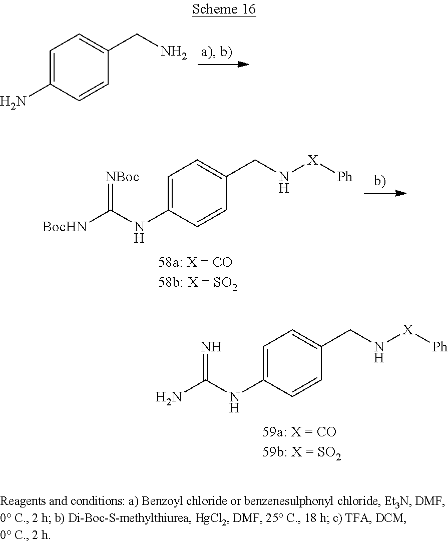

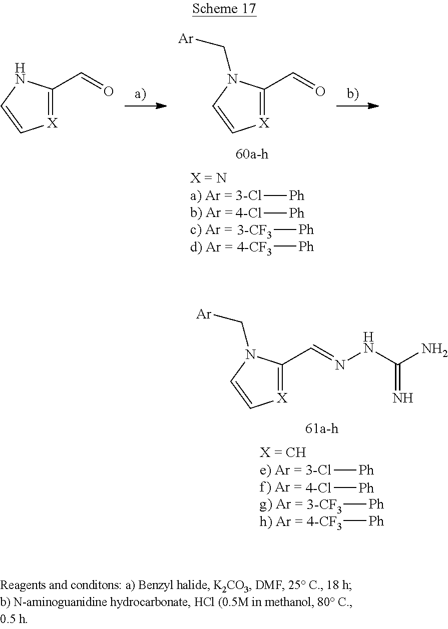

### Experimental Section

General Methods for Synthesis:

All chemicals were purchased from either Aldrich Chemical Co. (Gillingham, UK) or Alfa Aesar (Heysham, UK). All organic solvents of AR grade were supplied by Fisher Scientific (Loughborough, UK). Melting points were determined using a Stanford Research Systems Optimelt MPA100 and are uncorrected. Thin layer chromatography (TLC) was performed on pre-coated aluminum plates (Merck, silica gel 60 F₂₅₄). Products were visualized either by UV irradiation at 254 nm and by staining with 5% w/v molybdophosphoric acid in ethanol, followed by heating. Flash column chromatography was performed on pre-packed columns (RediSep Rf) and gradient elution (solvents indicated in text) on the Combiflash RF system (Teledyne Isco). ¹H NMR spectra were recorded with a Bruker 400 or 500 MHz spectrometer. Chemical shifts are reported in parts per million (ppm, δ) relative to tetramethylsilane (TMS) as an internal standard. High resolution mass spectra were recorded on a Bruker MicroTOF with ESI.

### General Procedure: Guanylation of substituted 3-(aminomethyl)phenol (1a-1c)

To a solution of the substituted 3-(aminomethyl)phenol hydrochloride (1a-1c) (6.0 mmol) in DMF (25 mL) was added S-methyl-N,N′-bis(tert-butoxycarbonyl) isothiourea (1.3 g, 4.5 mmol), followed by triethylamine (3.0 mL). The mixture was stirred at ambient temperature overnight, partitioned between ethyl acetate (100) ml and 5% citric acid (50 mL). The organic phase was washed with brine, dried over magnesium sulphate and concentrated in vacuo to give an off-white solid. Purification by flash chromatography eluting with gradient solvent (petrol ether to 45% ethyl acetate-petrol ether) afford the product (2a-2c).

### General Procedure: Synthesis of the Benzyl Guanidine Derivatives (4a-4p)

To a solution of the substituted N,N′-di-Boc-(guanydinomethyl)benzene (3a-3p) (0.3 mmol) in DCM (2 mL) was added trifluoroacetic acid (1 mL). The mixture was shaken at ambient temperature overnight, evaporated to dryness. Ethyl ether (1 mL) was added, and the precipitate was collected and washed with ether, dried in vacuo to give the target compound (4a-4p) as a white or off-white solid.

### 1-{[3-(benzyloxy)phenyl]methyl}guanidine 2,2,2-trifluoroacetate (4a)

A white solid was obtained (95% yield). ¹H NMR (400 MHz, CD₃OD) δ 7.33 (m, 2H, ArH), 7.24 (m, 2H, ArH), 7.18-7.29 (m, 2H, ArH), 6.80-6.95 (m, 2H, ArH), 4.98 (s, 2H, CH₂) and 4.26 (s, 2H, CH₂); HRMS (ESI) calcd. for C₁₅H₁₈N₃O (M+H)⁺ 256.1450. found 256.1454.

### 1-({3-[(4-chlorophenyl)methoxy]phenyl}methyl)guanidine 2,2,2-trifluoroacetate (4b)

An off-white solid was obtained (96% yield). ¹H NMR (400 MHz, CD₃OD) δ 7.43-7.50 (m, 4H, ArH), 7.37 (t, J=8.3 Hz, 1H, ArH), 6.98-7.05 (m, 3H, ArH), 5.16 (s, 2H, CH₂) and 4.39 (s, 2H, CH₂); HRMS (ESI) calcd. for C₁₅H₁₇ClN₃O (M+H)⁺ 290.1060. found 290.1060.

### 1-({3-[(3-chlorophenyl)methoxy]phenyl}methyl)guanidine 2,2,2-trifluoroacetate (4c)

An off-white solid was obtained (85% yield). ¹H NMR (400 MHz, CD₃OD) δ 7.51 (s, 1H, ArH), 7.32-7.40 (m, 4H, ArH), 6.98-7.05 (m, 3H, ArH), 5.16 (s, 2H, CH₂) and 4.38 (s, 2H, CH₂); HRMS (ESI) calcd. for C₁₅H₁₇ClN₃O (M+H)⁺ 290.1060. found 290.1067.

### 1-({3-[(2,4-dichlorophenyl)methoxy]phenyl}methyl)guanidine 2,2,2-trifluoroacetate (4d)

A white solid was obtained (98% yield). ¹H NMR (400 MHz, DMSO-d6) δ 8.05 (br s, 1H, NH), 7.78 (t, J=2.1 Hz, 1H, ArH), 7.67 (d, J=7.9 Hz, 1H, ArH), 7.55 (dd, J=7.9, 1.9 Hz, 1H, ArH), 7.38 (t, J=8.0 Hz, 1H, ArH), 6.95-7.05 (m, 3H, ArH), 5.20 (s, 2H, CH₂) and 4.40 (d, J=5.0 Hz, 2H, CH₂); HRMS (ESI) calcd. for C₁₅H₁₆Cl₂N₃O (M+H)⁺ 324.0670. found 324.0665.

### 1-({3-[(3,4-dichlorophenyl)methoxy]phenyl}methyl)guanidine 2,2,2-trifluoroacetate (4e)

A white solid was obtained (99% yield). ¹H NMR (400 MHz, DMSO-d6) δ 8.02 (br s, 1H, NH), 7.78 (t, J=1.9 Hz, 1H, ArH), 7.72 (d, J=8.1 Hz, 1H, ArH), 7.50 (dd, J=8.0, 1.9 Hz, 1H, ArH), 7.38 (td, J=7.9, 1.9 Hz, 1H, ArH), 6.93-7.05 (m, 3H, ArH), 5.19 (s, 2H, CH₂) and 4.39 (d, J=5.1 Hz, 2H, CH₂); HRMS (ESI) calcd. for C₁₅H₁₆Cl₂N₃O (M+H)⁺ 324.0670. found 324.0667.

### 1-({3-[(2,5-dichlorophenyl)methoxy]phenyl}methyl)guanidine 2,2,2-trifluoroacetate (4f)

A white solid was obtained (98% yield). ¹H NMR (400 MHz, DMSO-d6) δ 8.10 (br s, 1H, NH), 7.72 (t, J=2.1 Hz, 1H, ArH), 7.63 (d, J=8.0 Hz, 1H, ArH), 7.55 (dd, J=8.0, 2.1 Hz, 1H, ArH), 7.40 (td, J=8.0, 1.8 Hz, 1H, ArH), 6.95-7.08 (m, 3H, ArH), 5.20 (s, 2H, CH₂) and 4.42 (d, J=6.0 Hz, 2H, CH₂); HRMS (ESI) calcd. for C₁₅H₁₆Cl₂N₃O (M+H)⁺ 324.0670. found 324.0675.

### 1-({3-[(2,3-dichlorophenyl)methoxy]phenyl}methyl)guanidine 2,2,2-trifluoroacetate (4g)

A white solid was obtained (92% yield). ¹H NMR (400 MHz, DMSO-d6) δ 8.05 (br s, 1H, NH), 7.72 (dd, J=8.0, 1.8 Hz, 1H, ArH), 7.63 (dd, J=8.0, 1.8 Hz, 1H, ArH), 7.48 (t, J=8.0 Hz, 1H, ArH), 7.40 (td, J=8.0, 1.8 Hz, 1H, ArH), 6.95-7.08 (m, 3H, ArH), 5.25 (s, 2H, CH₂) and 4.40 (d, J=5.5 Hz, 2H, CH₂); HRMS (ESI) calcd. for C₁₅H₁₆Cl₂N₃O (M+H)⁺ 324.0670. found 324.0661.

### 1-({3-[(2,3-dichlorophenyl)methoxy]phenyl}methyl)guanidine hydrochloride (4g.HCl)

Compound 4 g (5 mg) was converted to the hydrogen chloride salt by dissolving in HCl-methanol (0.5 M, 2 mL) solution and concentrating in vacuo. A white solid was obtained (4 mg). HRMS (ESI) calcd. for C₁₅H₁₆Cl₂N₃O (M+H)⁺ 324.0670. found 324.0677.

### 1-[(3-{[4-(trifluoromethyl)phenyl]methoxy}phenyl)methyl]guanidine 2,2,2-trifluoroacetate (4h)

A white solid was obtained (97% yield). ¹H NMR (400 MHz, DMSO-d6) δ 8.05 (br s, 1H, NH), 7.82 (d, J=8.2 Hz, 2H, ArH), 7.72 (d, J=8.2 Hz, 2H, ArH), 7.38 (m, 2H, ArH), 6.95-7.12 (m, 2H, ArH), 5.25 (s, 2H, CH₂) and 4.40 (d, J=5.5 Hz, 2H, CH₂); HRMS (ESI) calcd. for C₂₆H₃₃F₃N₃O₅(M+H)⁺ 524.2372. found 524.2360.

### 1-[(3-{[3-(trifluoromethyl)phenyl]methoxy}phenyl)methyl]guanidine 2,2,2-trifluoroacetate (4i)

An off-white solid was obtained (98% yield). ¹H NMR (400 MHz, DMSO-d6) δ 8.05 (br s, 1H, NH), 7.87 (s, 1H, ArH), 7.82 (d, J=8.0 Hz, 1H, ArH), 7.78 (d, J=8.0 Hz, 1H, ArH), 7.71 (t, J=7.9 Hz, 1H, ArH), 7.13 (td, J=7.9, 1.7 Hz, 1H, ArH), 6.95-7.08 (m, 3H, ArH), 5.27 (s, 2H, CH₂) and 4.40 (d, J=6.1 Hz, 2H, CH₂); HRMS (ESI) calcd. for C₂₆H₃₃F₃N₃O₅(M+H)⁺ 524.2372. found 524.2397.

### 1-({3-[(4-bromophenyl)methoxy]phenyl}methyl)guanidine 2,2,2-trifluoroacetate (4j)

A white solid was obtained (97% yield). ¹H NMR (400 MHz, DMSO-d6) δ 8.10 (br s, 1H, NH), 7.65 (d, J=8.1 Hz, 2H, ArH), 7.46 (d, J=8.0 Hz, 2H, ArH), 7.37 (t, J=8.0 Hz, 1H, ArH), 6.95-7.08 (m, 3H, ArH), 5.14 (s, 2H, CH₂) and 4.39 (d, J=6.2 Hz, 2H, CH₂); HRMS (ESI) calcd. for C₁₅H₁₆BrN₃NaO (M+Na)⁺ 356.0374. found 356.0375.

### 1-({3-[(4-fluorophenyl)methoxy]phenyl}methyl)guanidine 2,2,2-trifluoroacetate (4k)

A white solid was obtained (90% yield). ¹H NMR (400 MHz, DMSO-d6) δ 8.07 (br s, 1H, NH), 7.60 (d, J=8.2 Hz, 2H, ArH), 7.45 (d, J=8.2 Hz, 2H, ArH), 7.35 (td, J=8.0, 1.5 Hz, 1H, ArH), 6.97-7.10 (m, 3H, ArH), 5.11 (s, 2H, CH₂) and 4.40 (d, J=6.2 Hz, 2H, CH₂); HRMS (ESI) calcd. for C₁₅H₁₇FN₃O (M+H)⁺ 274.1356. found 274.1366.

### 1-[(3-{[2-chloro-3-(trifluoromethyl)phenyl]methoxy}phenyl)methyl]guanidine 2,2,2-trifluoroacetate (4l)

A white solid was obtained (99% yield). ¹H NMR (400 MHz, DMSO-d6) δ 8.12 (br s, 1H, NH), 7.99 (d, J=7.9 Hz, 1H, ArH), 7.95 (d, J=7.9 Hz, 1H, ArH), 7.68 (t, J=8.1 Hz, 1H, ArH), 7.40 (t, J=8.0 Hz, 1H, ArH), 6.96-7.08 (m, 3H, ArH), 5.31 (s, 2H, CH₂) and 4.41 (d, J=6.1 Hz, 2H, CH₂); HRMS (ESI) calcd. for C₁₆H₁₆ClF₃N₃O (M+H)⁺ 358.0934. found 358.0979.

### 1-[(3-{[2-chloro-3-(trifluoromethyl)phenyl]methoxy}phenyl)methyl]guanidine hydrochloride (4l.HCl)

Compound 4l (9 mg) was converted to the hydrogen chloride salt by dissolving in HCl-methanol (0.5 M, 2 mL) solution and concentrating in vacuo. A white solid was obtained (7 mg). HRMS (ESI) calcd. for C₁₆H₁₆ClF₃N₃O (M+H)⁺ 358.0934. found 358.0897.

### 1-({3-[(3-chlorophenyl)methoxy]-4-methoxyphenyl}methyl)guanidine 2,2,2-trifluoroacetate (4m)

A white solid was obtained (97% yield). ¹H NMR (400 MHz, DMSO-d6) δ 8.12 (br s, 1H, NH), 7.65 (s, 1H, ArH), 7.39-7.45 (m, 3H, ArH), 6.90-7.15 (m, 3H, ArH), 5.27 (s, 2H, CH₂) and 4.36 (d, J=6.0 Hz, 2H, CH₂); HRMS (ESI) calcd. for C₁₆H₁₈ClN₃NaO₂ (M+Na)⁺ 342.0985. found 342.0977.

### 1-({3-[(4-chlorophenyl)methoxy]-4-methoxyphenyl}methyl)guanidine 2,2,2-trifluoroacetate (4n)

An off-white solid was obtained (95% yield). ¹H NMR (400 MHz, DMSO-d6) δ 8.07 (br s, 1H, NH), 7.30-7.39 (m, 4H, ArH), 6.87-7.02 (m, 3H, ArH), 5.29 (s, 2H, CH₂) and 4.40 (d, J=6.0 Hz, 2H, CH₂); HRMS (ESI) calcd. for C₁₆H₁₈ClN₃NaO₂ (M+Na)⁺ 342.0985. found 342.0994.

### 1-({3-[(2,3-dichlorophenyl)methoxy]-2,6-difluorophenyl}methyl)guanidine 2,2,2-trifluoroacetate (4o)

A white solid was obtained (93% yield). ¹H NMR (400 MHz, DMSO-d6) δ 7.96 (br s, 1H, NH), 7.75 (d, J=8.0 Hz, 1H, ArH), 7.63 (d, J=7.9 Hz, 1H, ArH), 7.50 (t, J=7.9 Hz, 1H, ArH), 7.30-7.40 (m, 2H, ArH), 5.35 (s, 2H, CH₂) and 4.50 (s, 2H, CH₂); HRMS (ESI) calcd. for C₁₅H₁₄Cl₂F₂N₃O (M+H)⁺ 360.0482. found 360.0493.

### 1-[(2,6-difluoro-3-{[3-(trifluoromethyl)phenyl]methoxy}phenyl)methyl]guanidine 2,2,2-trifluoroacetate (4p)

A white solid was obtained (98% yield). ¹H NMR (400 MHz, DMSO-d6) δ 8.00 (br s, 1H, NH), 7.88 (s, 1H, ArH), 7.81 (d, J=7.9 Hz, 1H, ArH), 7.72 (t, J=8.1 Hz, 1H, ArH), 7.32-7.40 (m, 2H, ArH), 7.18 (dt, J=7.9, 1.6 Hz, 1H, ArH), 5.35 (s, 2H, CH₂) and 4.49 (d, J=5.3 Hz, 2H, CH₂); HRMS (ESI) calcd. for C₁₆H₁₅F₅N₃O (M+H)⁺ 360.1135. found 360.1248.

### ({3-[(2,3-dichlorophenyl)methoxy]phenyl}methyl)(methyl)amine (7a)

To a solution of 3-[(2,3-dichlorophenyl)methoxy]benzaldehyde (330 mg, 1.17 mmol) in methanol (5 mL) was added a methylamine-methanol solution (2.0 M, 0.76 mL, 1.5 mmol). The mixture was stirred at ambient temperature for 10 hr, cooled to 0° C. Sodium borohydride (53 mg, 1.4 mmol) was added slowly. The mixture was stirred at room temperature for 4 hours, partitioned between 1N NaOH and EtOAc. The organic phase was washed with brine, dried over magnesium sulphate and concentrated in vacuo to give clear oil (340 mg, 98%). ¹H NMR (400 MHz, CDCl₃) δ 7.47 (d, J=7.9 Hz, 1H, ArH), 7.40 (d, J=7.9 Hz, 1H, ArH), 7.18-7.28 (m, 2H, ArH), 6.92-7.00 (m, 2H, ArH), 6.85 (dd, J=8.0, 1.5 Hz, 1H, ArH), 5.15 (s, 2H, CH₂), 3.74 (s, 2H, CH₂) and 2.45 (s, 3H, CH₃); HRMS (ESI) calcd. for C₁₅H₁₆Cl₂NO (M+H)⁺ 296.0609. found 296.0680.

### ({3-[(2,3-dichlorophenyl)methoxy]phenyl}methyl)(2-methoxyethyl)amine (7b)

To a solution of 3-[(2,3-dichlorophenyl)methoxy]benzaldehyde (330 mg, 1.17 mmol) in methanol (5 mL) was added 2-methoxyethan-1-amine (113 mg, 1.5 mmol). The mixture was stirred at ambient temperature for 10 hr, cooled to 0° C. Sodium borohydride (57 mg, 1.5 mmol) was added slowly. The mixture was stirred at room temperature for 4 hours, partitioned between 1N NaOH and EtOAc. The organic phase was washed with brine, dried over magnesium sulphate and concentrated in vacuo to give clear oil (350 mg, 88%). ¹H NMR (400 MHz, CDCl₃) δ 7.48 (d, J=7.9 Hz, 1H, ArH), 7.43 (d, J=8.0 Hz, 1H, ArH), 7.20-7.26 (m, 2H, ArH), 6.99 (s, 1H, Arh), 6.96 (d, J=7.5 Hz, 1H, ArH), 6.85 (dd, J=7.9, 2.6 Hz, 1H, ArH), 5.16 (s, 2H, CH₂), 3.80 (s, 2H, CH₂), 3.51 (t, J=5.3 Hz, 2H, ArH), 3.35 (s, 3H, CH₃) and 2.80 (t, J=5.2 Hz, 2H, ArH); HRMS (ESI) calcd. for C₁₇H₂₀Cl₂NO₂ (M+H)⁺ 340.0871. found 340.0848.

### tert-butyl N-[{[(tert-butoxy)carbonyl]imino}[({3-[(2,3-dichlorophenyl) methoxy]phenyl}methyl)(methyl)amino]methyl]carbamate (8a)

To a solution of ({3-[(2,3-dichlorophenyl)methoxy]phenyl}methyl)(methyl)amine (7a, 330 mg, 1.11 mmol) in DMF (5 mL) was added S-methyl-N,N′-bis(tert-butoxycarbonyl)isothiourea (389 mg, 1.3 mmol), followed by triethylamine (0.3 mL) and HgCl₂ (200 mg). The mixture was stirred at ambient temperature overnight, diluted with EtOAc (50 mL), and filtered through Celite. The organic phase was washed with brine, dried over magnesium sulphate and concentrated in vacuo to give colourless oil. Purification by flash chromatography eluting with gradient solvent (petrol ether to 20% ethyl acetate-petrol ether) afford the product (8a) as a white solid (350 mg, 59% yield). mp 127-129° C. ¹H NMR (400 MHz, CDCl₃) δ 10.2 (s, 1H, NH), 7.49 (d, J=7.9 Hz, 1H, ArH), 7.41 (d, J=7.9 Hz, 1H, ArH), 7.22-7.28 (m, 3H, ArH), 6.88-6.97 (m, 2H, ArH), 5.16 (s, 2H, CH₂), 4.70 (s, 2H, CH₂), 2.90 (s, 3H, CH₃) and 1.50 (s, 18H); HRMS (ESI) calcd. for C₂₆H₃₃Cl₂N₃NaO₅ (M+Na)⁺ 560.1695. found 560.1697.

### tert-butyl N-[{[(tert-butoxy)carbonyl]imino}[({3-[(2,3-dichlorophenyl) methoxy]phenyl}methyl)(2-methoxyethyl)amino]methyl]carbamate (8b)

To a solution of ({3-[(2,3-dichlorophenyl)methoxy]phenyl}methyl)(2-methoxyethyl)amine (7b, 320 mg, 0.94 mmol) in DMF (3 mL) was added S-methyl-N,N′-bis(tert-butoxycarbonyl)isothiourea (330 mg, 1.13 mmol), followed by triethylamine (0.5 mL) and HgCl₂ (306 mg). The mixture was stirred at ambient temperature overnight, diluted with EtOAc (50 mL), and filtered through Celite. The organic phase was washed with brine, dried over magnesium sulphate and concentrated in vacuo to give colourless oil. Purification by flash chromatography eluting with gradient solvent (petrol ether to 20% ethyl acetate-petrol ether) afford the product as a white solid (340 mg, 62% yield). mp 125-126° C. ¹H NMR (400 MHz, CDCl₃) δ 9.56 (s, 1H, NH), 7.49 (d, J=8.2 Hz, 1H, ArH), 7.43 (d, J=8.3 Hz, 1H, ArH), 7.21-7.28 (m, 3H, ArH), 6.86-6.97 (m, 2H, ArH), 5.20 (s, 2H, CH₂), 4.89 (br s, 2H, CH₂), 3.65 (br s, 2H, CH₂), 3.45 (s, 2H, CH₂), 3.30 (s, 3H, CH₃) and 1.50 (s, 18H); HRMS (ESI) calcd. for C₂₈H₃₈Cl₂N₃O₆ (M+H)⁺ 582.2138. found 582.2177.

### 1-({3-[(2,3-dichlorophenyl)methoxy]phenyl}methyl)-1-methylguanidine hydrochloride (9a)

To a solution of the intermediate 8a (126 mg, 0.23 mmol) in DCM (1.5 mL) was added trifluoroacetic acid (0.75 mL). The mixture was shaken at ambient temperature overnight, evaporated to dryness. Ethyl ether (1 mL) was added, and the precipitate was collected and washed with ether, dried in vacuo to give the compound (9a) as a white solid (69 mg, 87%). The compound 9a (18 mg) was dissolved in 0.5M HCl-methanol solution (5 mL), and concentrated to give a white solid (15 mg). ¹H NMR (400 MHz, CDCl₃) δ 7.49 (br s, 2H, ArH), 7.18-7.36 (m, 2H, ArH), 6.98 (d, J=8.2 Hz, 1H, ArH), 6.76-6.86 (m, 2H, ArH), 5.18 (s, 2H, CH₂), 4.56 (br s, 2H, CH₂) and 2.96 (s, 3H, CH₃); HRMS (ESI) calcd. for C₁₆H₁₈Cl₂N₃O (M+H)⁺ 338.0827. found 338.0909.

### 1-({3-[(2,3-dichlorophenyl)methoxy]phenyl}methyl)-1-(2-methoxyethyl) guanidine 2,2,2-trifluoroacetate (9b)

To a solution of the intermediate 8b (98 mg, 0.17 mmol) in DCM (1.5 mL) was added trifluoroacetic acid (0.75 mL). The mixture was shaken at ambient temperature overnight, evaporated to dryness. Ethyl ether (1 mL) was added, and the precipitate was collected and washed with ether, dried in vacuo to give the compound (9b) as a white solid (70 mg, 86%). ¹H NMR (400 MHz, CDCl₃) δ 7.73 (dd, J=8.1, 1.7 Hz, 1H, ArH), 7.62 (dd, J=8.1, 1.8 Hz, 1H, ArH), 7.37-7.50 (m, 2H, ArH), 7.06 (dd, J=7.9, 2.1 Hz, 1H, ArH), 6.85-6.95 (m, 2H, ArH), 5.26 (s, 2H, CH₂), 3.56 (m, 4H), 4.67 (br s, 2H, CH₂) and 3.31 (s, 3H, CH₃); HRMS (ESI) calcd. for C₁₈H₂₂Cl₂N₃O₂ (M+H)⁺ 382.1089. found 382.1195.

### {3-[(2,3-dichlorophenyl)methoxy]phenyl}methanol (11)

The solution of 3-[(2,3-dichlorophenyl)methoxy]benzaldehyde (5, 1.3 g, 4.6 mmol) in EtOH (20 mL) and THF (5 mL) was cooled to 0° C. Sodium borohydride (176 mg, 4.6 mmol) was added portionwise. The mixture was stirred at ambient temperature overnight. Acetone (2 mL) was added, and the mixture was partitioned between EtOAc and saturated NH₄Cl solution. The organic phase was washed with brine, dried over magnesium sulphate and concentrated in vacuo to give a white solid (1.2 g, 92% yield). mp 79-80° C. ¹H NMR (400 MHz, CDCl₃) δ 7.49 (d, J=8.2 Hz, 1H, ArH), 7.43 (d, J=8.2 Hz, 1H, ArH), 7.20-7.31 (m, 2H, ArH), 6.96-7.03 (m, 2H, ArH), 6.90 (dd, J=8.0, 1.9 Hz, 1H, ArH), 5.24 (s, 2H, CH₂) and 4.64 (s, 2H, CH₂).

### 1,2-dichloro-3-[3-(chloromethyl)phenoxymethyl]benzene (12)

To a solution of compound 11 (1.1 g, 3.9 mmol) in DCM (20 mL) was added triethylamine (1.1 mL, 7.8 mmol), followed by methanesulfonyl chloride (0.6 mL, 7.8 mmol). The mixture was stirred at ambient temperature overnight, and partitioned between DCM and saturated NaHCO₃ solution. The organic phase was washed with brine, dried over magnesium sulphate and concentrated in vacuo to give a white solid (0.95 g, 91% yield). mp 91-95° C. ¹H NMR (400 MHz, CDCl₃) δ 7.49 (d, J=8.1 Hz, 1H, ArH), 7.44 (d, J=8.1 Hz, 1H, ArH), 7.21-7.32 (m, 2H, ArH), 7.00-7.05 (m, 2H, ArH), 6.92 (dd, J=8.1, 1.7 Hz, 1H, ArH), 5.17 (s, 2H, CH₂) and 4.53 (s, 2H, CH₂).

### tert-butyl N-[{[(tert-butoxy)carbonyl]({3-[(2,3-dichlorophenyl)methoxy]phenyl}methyl)amino}(methylsulfanyl)methylidene]carbamate (13)

To a solution of 12 (330 mg, 1.1 mmol) in DCM (5 mL) were added KOH (112 mg, 2.2 mmol), water (5 mL) and Bu₄NHSO4 (34 mg, 0.1 mmol). The mixture was stirred at ambient temperature overnight, and partitioned between DCM and water. The organic phase was washed with brine, dried over magnesium sulphate and concentrated in vacuo to give clear oil (300 mg, 49% yield). ¹H NMR (400 MHz, CDCl₃) δ 7.47 (d, J=8.1 Hz, 1H, ArH), 7.38 (d, J=8.2 Hz, 1H, ArH), 7.18-7.26 (m, 2H, ArH), 6.95-7.01 (m, 2H, ArH), 6.86 (dd, J=7.9, 1.7 Hz, 1H, ArH), 5.15 (s, 2H, CH₂), 4.75 (s, 2H, CH₂), 2.40 (s, 3H, CH₃), 1.51 (s, 9H) and 1.39 (s, 9H); HRMS (ESI) calcd. for C₂₆H₃₂Cl₂N₂NaO₅S (M+Na)⁺ 577.1306. found 577.1303.

### tert-butyl N-[{[(tert-butoxy)carbonyl]({3-[(2,3-dichlorophenyl)methoxy]phenyl}methyl)amino}(methylamino)methylidene]carbamate (14)

To a solution of 13 (200 mg, 0.36 mmol) in DMF (1 mL) was added methylamine-methanol solution (2M, 0.27 mL, 0.54 mmol), followed by triethylamine (0.2 mL) and HgCl₂ (116 mg). The mixture was stirred at ambient temperature for 1.5 hr, diluted with EtOAc (30 mL), and filtered through Celite. The organic phase was washed with brine, dried over magnesium sulphate and concentrated in vacuo to give colourless oil. Purification by flash chromatography eluting with gradient solvent (petrol ether to 20% ethyl acetate-petrol ether) afford the product (8a) as a white solid (100 mg, 52% yield). mp 111-113° C. ¹H NMR (400 MHz, CDCl₃) δ 9.87 (s, 1H, NH), 7.49 (d, J=8.0 Hz, 1H, ArH), 7.43 (d, J=7.9 Hz, 1H, ArH), 7.19-7.26 (m, 2H, ArH), 6.87-6.96 (m, 3H, ArH), 5.15 (s, 2H, CH₂), 4.57 (s, 2H, CH₂), 2.52 (s, 3H, CH₃), 1.51 (s, 9H) and 1.46 (s, 9H).

### 3-({3-[(2,3-dichlorophenyl)methoxy]phenyl}methyl)-1-methylguanidine 2,2,2-trifluoroacetate (15)

To a solution of the intermediate 14 (85 mg, 0.16 mmol) in DCM (1 mL) was added trifluoroacetic acid (0.5 mL). The mixture was shaken at ambient temperature overnight, evaporated to dryness. The residue was dissolved in methanol, and evaporated to give a foamy powder (60 mg, 87%). ¹H NMR (400 MHz, CDCl₃) δ 7.52 (d, J=7.6 Hz, 2H, ArH), 7.24-7.33 (m, 2H, ArH), 6.88-6.95 (m, 3H, ArH), 5.19 (s, 2H, CH₂), 4.39 (s, 2H) and 2.85 (s, 3H, CH₃); HRMS (ESI) calcd. for C₁₆H₁₈Cl₂N₃O (M+H)⁺ 338.0827. found 338.0945.

### tert-Butyl N-[{[(tert-butoxy)carbonyl]imino}({[(4-hydroxyphenyl)methyl]amino}) methyl]carbamate (17)

To a solution of 4-(aminomethyl)phenol hydrochloride (6.0 mmol) in DMF (20 mL) was added S-methyl-N,N′-bis(tert-butoxycarbonyl)isothiourea (1.6 g, 5.6 mmol), followed by triethylamine (2.0 mL). The mixture was stirred at ambient temperature overnight, partitioned between ethyl acetate (100) ml and 5% citric acid (50 mL). The organic phase was washed with brine, dried over magnesium sulphate and concentrated in vacuo to give an off-white solid. Purification by flash chromatography eluting with gradient solvent (petrol ether to 45% ethyl acetate-petrol ether) afford the product (17) as a white solid (1.4 g, 68% yield). mp 137-138° C. ¹H NMR (400 MHz, CDCl₃) δ 11.5 (s, 1H, NH), 8.50 (br s, 1H, NH), 7.10 (d, J=8.1 Hz, 2H, ArH), 6.91 (d, J=8.1 Hz, 2H, ArH), 4.51 (d, J=5.2 Hz, 2H, CH₂), 1.49 (s, 9H) and 1.47 (s, 9H); HRMS (ESI) calcd. for C₁₈H₂₈N₃O₅ (M+H)⁺ 366.2029. found 366.2037.

### General Procedure: Benzylation of 4-(N,N′-di-Boc-guanydinomethyl)phenol (17)

To a solution of 4-(N,N′-di-Boc-guanydinomethyl)phenol (0.56 mmol) (17) in acetone (8 mL) was added the substituted benzyl bromide (0.56 mmol), followed by potassium carbonate (96 mg). The mixture was stirred at ambient temperature overnight, partitioned between ethyl acetate (50) ml and water (50 mL). The organic phase was washed with brine, dried over magnesium sulphate and concentrated in vacuo to give an off-white solid. Purification by flash chromatography eluting with gradient solvent (petrol ether to 25% ethyl acetate-petrol ether) afford the product (18a-18e).

### tert-butyl N-[({[4-(benzyloxy)phenyl]methyl}amino)({[(tert-butoxy) carbonyl]amino})methylidene]carbamate (18a)

A white solid was obtained (82% yield). mp 115-116° C. ¹H NMR (400 MHz, CDCl₃) δ 10.5 (br s, 1H, NH), 9.30 (br s, 1H, NH), 7.28-7.44 (m, 5H, ArH), 7.19 (dt, J=8.8, 2.1 Hz, 2H, ArH), 6.88 (dt, J=8.8, 2.0 Hz, 2H, ArH), 5.10 (s, 2H), 5.15 (br s, 2H), 1.49 (s, 9H) and 1.34 (s, 9H); HRMS (ESI) calcd. for C₂₅H₃₃N₃NaO₅ (M+Na)⁺ 478.2318. found 478.2329.

### tert-butyl N-[{[(tertbutoxy)carbonyl]amino}[({4-[(4-chlorophenyl)methoxy]phenyl}methyl)amino]methylidene]carbamate (18b)

A white solid was obtained (79% yield). mp 120-121° C. ¹H NMR (400 MHz, CDCl₃) δ 11.5 (br s, 1H, NH), 8.75 (br s, 1H, NH), 7.35 (s, 4H, ArH), 7.25 (d, J=8.2 Hz, 2H, ArH), 6.92 (d, J=8.2 Hz, 2H, ArH), 5.01 (s, 2H), 4.65 (br s, 2H), 1.52 (s, 9H) and 1.47 (s, 9H).

### tert-butyl N-[{[(tertbutoxy)carbonyl]amino}[({4-[(3-chlorophenyl)methoxy]phenyl}methyl)amino]methylidene]carbamate (18c)

A white solid was obtained (67% yield). mp 120-122° C. ¹H NMR (400 MHz, CDCl₃) δ 11.5 (br s, 1H, NH), 8.62 (br s, 1H, NH), 7.43 (s, 1H, ArH), 7.20-7.30 (m, 5H, ArH), 6.93 (dd, J=7.1, 2.0 Hz, 2H, ArH), 5.02 (s, 2H), 4.60 (br s, 2H), 1.52 (s, 9H) and 1.47 (s, 9H).

### tert-butyl N-[{[(tertbutoxy)carbonyl]amino}[({4-[(3,4-dichlorophenyl)methoxy]phenyl}methyl)amino]methylidene]carbamate (18d)

A white solid was obtained (79% yield). mp 156-158° C. ¹H NMR (400 MHz, CDCl₃) δ 11.5 (br s, 1H, NH), 8.80 (br s, 1H, NH), 7.51 (d, J=1.6 Hz, 1H, ArH), 7.43 (d, J=8.1 Hz, 1H, ArH), 7.22 (d, J=7.9 Hz, 1H, ArH), 6.91 (d, J=7.9 Hz, 2H, ArH), 5.00 (s, 2H), 4.63 (br s, 2H), 1.52 (s, 9H) and 1.47 (s, 9H).

### tert-butyl N-[{[(tertbutoxy)carbonyl]amino}[({4-[(2,3-dichlorophenyl)methoxy]phenyl}methyl)amino]methylidene]carbamate (18e)

A white solid was obtained (89% yield). mp 99-101° C. ¹H NMR (400 MHz, CDCl₃) δ 11.6 (br s, 1H, NH), 8.90 (br s, 1H, NH), 7.47 (d, J=8.2 Hz, 1H, ArH), 7.43 (d, J=8.2 Hz, 1H, ArH), 7.25-7.31 (m, 3H, ArH), 6.95 (d, J=8.1 Hz, 2H, ArH), 5.16 (s, 2H), 4.73 (br s, 2H), 1.53 (s, 9H) and 1.48 (s, 9H).

### General Procedure: Synthesis of the Benzyl Guanidine Derivatives (19a-19e)

To a solution of the para-substituted N,N′-di-Boc-(4-guanydinomethyl)benzene (100 mg) (18a-18e) in DCM (2 mL) was added trifluoroacetic acid (1 mL). The mixture was shaken at ambient temperature overnight, evaporated to dryness. Ethyl ether (1 mL) was added, and the precipitate was collected and washed with ether, dried in vacuo to give the target compound (19a-19e) as a white or off-white solid.

### 1-(4-(benzyloxy)benzyl)guanidine 2,2,2-trifluoroacetate (19a)

A off-white solid was obtained (96% yield). ¹H NMR (400 MHz, CD₃OD) δ 7.50 (d, J=8.2 Hz, 2H, ArH), 7.36-7.43 (m, 3H, ArH), 7.32 (d, J=8.3 Hz, 2H, ArH), 7.06 (d, J=8.3 Hz, 2H, ArH), 5.16 (s, 2H) and 4.38 (br s, 2H); HRMS (ESI) calcd. for C₁₅H₁₈N₃O (M+H)⁺ 256.1450. found 256.1539.

### 1-(4-((4-chlorobenzyl)oxy)benzyl)guanidine 2,2,2-trifluoroacetate (19b)

A white solid was obtained (87% yield). ¹H NMR (400 MHz, DMSO-d6) δ 8.15 (br s, 1H, NH), 7.37-7.41 (m, 4H, ArH), 7.27 (d, J=8.1 Hz, 2H, ArH), 6.94 (d, J=8.2 Hz, 2H, ArH), 5.17 (s, 2H) and 4.55 (br s, 2H); HRMS (ESI) calcd. for C₁₅H₁₇ClN₃O (M+H)⁺ 290.1060. found 290.1066.

### 1-(4-((3-chlorobenzyl)oxy)benzyl)guanidine 2,2,2-trifluoroacetate (19c)

A white solid was obtained (93% yield). ¹H NMR (400 MHz, DMSO-d6) δ 7.95 (br s, 1H, NH), 7.40-7.50 (m, 3H, ArH), 7.30 (m, 2H, ArH), 7.08 (m, 3H, ArH), 5.19 (s, 2H) and 4.33 (br s, 2H); HRMS (ESI) calcd. for C₁₅H₁₇ClN₃O (M+H)⁺ 290.1060. found 290.1071.

### 1-(4-((3,4-dichlorobenzyl)oxy)benzyl)guanidine 2,2,2-trifluoroacetate (19d)

A white solid was obtained (93% yield). ¹H NMR (400 MHz, DMSO-d6) δ 7.87 (br s, 1H, NH), 7.77 (d, J=2.0 Hz, 1H, ArH), 7.72 (d, J=7.7 Hz, 1H, ArH), 7.50 (dd, J=7.8, 2.1 Hz, 1H, ArH), 7.31 (d, J=8.5 Hz, 2H, ArH), 7.09 (d, J=8.6 Hz, 2H, ArH), 5.19 (s, 2H) and 4.33 (d, J=6.0 Hz, 2H); HRMS (ESI) calcd. for C₁₅H₁₅Cl₂N₃O (M+H)⁺ 324.0670. found 324.0658.

### 1-(4-((2,3-dichlorobenzyl)oxy)benzyl)guanidine 2,2,2-trifluoroacetate (19e)

A white solid was obtained (98% yield). ¹H NMR (400 MHz, DMSO-d6) δ 8.05 (br s, 1H, NH), 7.72 (d, J=7.9 Hz, 1H, ArH), 7.63 (d, J=8.1 Hz, 1H, ArH), 7.46 (t, J=7.9 Hz, 1H, ArH), 7.32 (d, J=8.3 Hz, 2H, ArH), 7.14 (d, J=8.2 Hz, 2H, ArH), 5.35 (s, 2H) and 4.38 (br s, 2H); HRMS (ESI) calcd. for C₁₅H₁₅Cl₂N₃O (M+H)⁺ 324.0670. found 324.0720.

### tert-butyl N-[{[(tert-butoxy)carbonyl]imino}[(4-hydroxyphenyl)amino] methyl]carbamate (21)

To a solution of 4-aminophenol (273 mg, 2.5 mmol) in DMF (6 mL) was added S-methyl-N,N′-bis(tert-butoxycarbonyl)isothiourea (690 mg, 2.37 mmol), followed by triethylamine (0.7 mL) and HgCl₂ (640 mg, 2.37 mmol). The mixture was stirred at ambient temperature overnight, diluted with EtOAc (30 mL), and filtered through Celite. The organic phase was washed with brine, dried over magnesium sulphate and concentrated in vacuo. Purification by flash chromatography eluting with gradient solvent (petrol ether to 40% ethyl acetate-petrol ether) afford the product as a white solid (535 mg, 64% yield). mp 182-184° C. ¹H NMR (400 MHz, CDCl₃) δ 11.6 (s, 1H, NH), 9.93 (s, 1H, NH), 7.02 (br s, 3H, ArH and OH), 6.56 (br s, 2H, ArH), 1.47 (s, 9H) and 1.44 (s, 9H); HRMS (ESI) calcd. for C₁₇H₂₅N₃NaO₅ (M+Na)⁺ 374.1692. found 374.1697.

### General Procedure: Benzylation of 4-(N,N′-di-Boc-guanydino)phenol (21)

To a solution of 4-(N,N′-di-Boc-guanydino)phenol (110 mg, 0.531 mmol) (21) in acetone (5 mL) was added the substituted benzyl bromide (0.37 mmol), followed by potassium carbonate (72 mg). The mixture was stirred at ambient temperature overnight, partitioned between ethyl acetate (30) ml and water (30 mL). The organic phase was washed with brine, dried over magnesium sulphate and concentrated in vacuo to give an off-white solid. Purification by flash chromatography eluting with gradient solvent (petrol ether to 25% ethyl acetate-petrol ether) afford the product (22a-22c).

### tert-butyl N-[{[4-(benzyloxy)phenyl]amino}({[(tert-butoxy)carbonyl]imino}) methyl]carbamate (22a)

A white solid was obtained (79% yield). mp 141-143° C. ¹H NMR (400 MHz, CDCl₃) δ 11.6 (s, 1H, NH), 10.2 (br s, 1H, NH), 7.47 (d, J=8.3 Hz, 2H, ArH), 7.30-7.42 (m, 5H, ArH), 6.92 (d, J=8.2 Hz, 2H, ArH), 5.05 (s, 2H), 1.52 (s, 9H) and 1.48 (s, 9H); HRMS (ESI) calcd. for C₂₄H₃₁N₃NaO₅ (M+Na)⁺ 464.2161. found 464.2179.

### tert-butyl N-[{[(tert-butoxy)carbonyl]imino}({4-[(4-chlorophenyl)methoxy]phenyl}amino)methyl]carbamate (22b)

A white solid was obtained (89% yield). mp 95-97° C. ¹H NMR (400 MHz, CDCl₃) δ 11.6 (s, 1H, NH), 10.2 (br s, 1H, NH), 7.51 (dd, J=7.8, 2.4 Hz, 2H, ArH), 7.30 (s, 4H, ArH), 6.91 (dd, J=7.8, 2.4 Hz, 2H, ArH), 5.00 (s, 2H), 1.52 (s, 9H) and 1.48 (s, 9H).

### tert-butyl N-[{[3-(benzyloxy)phenyl]amino}({[(tert-butoxy)carbonyl]imino}) methyl]carbamate (22c)

A white solid was obtained (62% yield). mp 79-83° C. ¹H NMR (400 MHz, CDCl₃) δ 11.8 (s, 1H, NH), 10.8 (br s, 1H, NH), 7.40 (d, J=8.3 Hz, 2H, ArH), 7.25-7.36 (m, 4H, ArH), 6.92 (d, J=8.4 Hz, 2H, ArH), 5.05 (s, 2H), 1.51 (s, 9H) and 1.47 (s, 9H).

### General Procedure: Synthesis of the Phenyl Guanidine Derivatives (23a-23c)

To a solution of the para-substituted N,N′-di-Boc-(4-guanydino)benzene (100 mg) (22a-22c) in DCM (2 mL) was added trifluoroacetic acid (1 mL). The mixture was shaken at ambient temperature overnight, evaporated to dryness. Ethyl ether (1 mL) was added, and the precipitate was collected and washed with ether, dried in vacuo to give the target compound (23a-23c) as a white or off-white solid.

### 1-[4-(benzyloxy)phenyl]guanidine 2,2,2-trifluoroacetate (23a)

A white solid was obtained (95% yield). ¹H NMR (400 MHz, CD₃OD) δ 7.46 (d, J=8.3 Hz, 2H, ArH), 7.32-7.40 (m, 3H, ArH), 7.22 (d, J=8.1 Hz, 2H, ArH), 7.12 (d, J=8.1 Hz, 2H, ArH) and 5.12 (s, 2H); HRMS (ESI) calcd. for C₁₄H₁₆N₃O (M+H)⁺ 242.1293. found 242.1283.

### 1-{4-[(4-chlorophenyl)methoxy]phenyl}guanidine 2,2,2-trifluoroacetate (23b)

A white solid was obtained (88% yield). ¹H NMR (400 MHz, CD₃OD) δ 7.55 (d, J=8.2 Hz, 2H, ArH), 7.37 (d, J=8.2 Hz, 2H, ArH), 7.21-7.30 (m, 4H, ArH) and 5.12 (s, 2H); HRMS (ESI) calcd. for C₁₄H₁₅ClN₃O (M+H)⁺ 276.0904. found 276.0932.

### 1-{4-[(3-chlorophenyl)methoxy]phenyl}guanidine 2,2,2-trifluoroacetate (23c)

A white solid was obtained (92% yield). ¹H NMR (400 MHz, CD₃OD) δ 7.59 (d, J=8.3 Hz, 2H, ArH), 7.47-7.53 (m, 3H, ArH), 7.38 (s, 1H, ArH), 7.32 (d, J=8.0 Hz, 2H, ArH) and 5.21 (s, 2H); HRMS (ESI) calcd. for C₁₄H₁₅ClN₃O (M+H)⁺ 276.0904. found 276.0911.

### 1-(5,6,7,8-tetrahydronaphthalen-1-yl)guanidine 2,2,2-trifluoroacetate (23d)

A white solid was obtained (95% yield). mp 153-155° C.; ¹H NMR (400 MHz, CD₃OD) δ 7.28 (br s, 2H), 6.95-7.05 (m, 2H, ArH), 6.88 (t, J=8.6 Hz, 1H, ArH), 4.00 (br s, 2H), 2.52 (d, J=5.0 Hz, 2H) and 2.51 (d, J=5.0 Hz, 2H); HRMS (ESI) calcd. for C₁₁H₁₆N₃(M+H)⁺ 190.1344. found 190.1438.

### tert-butyl N-[(7-bromo-1,2,3,4-tetrahydroisoquinolin-2-yl)({[(tert-butoxy) carbonyl]amino})methylidene]carbamate (25)

To a solution of 7-bromo-1,2,3,4-tetrahydroisoquinoline (318 mg, 1.5 mmol) in DMF (5 mL) was added S-methyl-N,N′-bis(tert-butoxycarbonyl)isothiourea (436 g, 1.5 mmol), followed by triethylamine (0.5 mL). The mixture was stirred at ambient temperature for 4 h, partitioned between ethyl acetate (100) ml and 5% citric acid (50 mL). The organic phase was washed with brine, dried over magnesium sulphate and concentrated in vacuo to give an off-white solid. Purification by flash chromatography eluting with gradient solvent (petrol ether to 25% ethyl acetate-petrol ether) afford a white solid (500 g, 73% yield). mp 131-132° C. ¹H NMR (400 MHz, CDCl₃) δ 10.3 (s, 1H, NH), 7.28 (dd, J=8.2, 1.9 Hz, 1H, ArH), 7.23 (br s, 1H, ArH), 7.01 (d, J=8.0 Hz, 1H, ArH), 4.70 (s, 2H, CH₂), 3.87 (t, J=5.7 Hz, 2H, CH₂), 2.90 (t, J=5.5 Hz, 2H, CH₂) and 1.50 (s, 18H); HRMS (ESI) calcd. for C₂₀H₂₉BrN₃O₄(M+H)⁺ 454.1341. found 454.1356.

### 7-bromo-1,2,3,4-tetrahydroisoquinoline-2-carboximidamid 2,2,2-trifluoroacetate (26)

To a solution of 25 (90 mg, 0.13 mmol) in DCM (0.5 mL) was added trifluoroacetic acid (0.5 mL). The mixture was shaken at ambient temperature overnight, evaporated to dryness. Ethyl ether (1 mL) was added, and the precipitate was collected and washed with ether, dried in vacuo to give the target compound 26 (45 mg, 94% yield) as a white solid. ¹H NMR (400 MHz, CD3OD) δ 7.47 (dd, J=8.3, 1.9 Hz, 1H, ArH), 7.46 (br s, 1H, ArH), 7.25 (d, J=8.2 Hz, 1H, ArH), 5.00 (s, 2H), 3.70 (t, J=5.3 Hz, 2H, CH₂), 3.00 (t, J=5.5 Hz, 2H, CH₂); HRMS (ESI) calcd. for C₁₀H₁₃BrN₃ (M+H)⁺ 254.0292. found 254.0282.

### tert-butyl N-[7-(4-tert-butylphenyl)-1,2,3,4-tetrahydroisoquinoline-2-carboximidoyl]carbamate (27)

To a solution of 25 (400 mg, 0.88 mmol) in dioxane/water (6 mL/2 mL) were added 4-t-butyl phenylboronic acid (188 mg, 1.05 mmol), K₂CO₃ (242 mg, 1.76 mmol). The mixture was degassed under vacuum for 1 minute. Pd(PPh₃)₄ (20 mg) was added to the solution. The mixture was stirred at 100° C. under nitrogen for 4 h, partitioned between ethyl acetate (50) ml and water (50 mL). The organic phase was washed with brine, dried over magnesium sulphate and concentrated in vacuo. The crude product was purified by flash chromatography eluting with 10% methanol-DCM to give clear oil (190 mg, 53% yield). ¹H NMR (400 MHz, CDCl₃) δ 10.1 (s, 1H, NH), 7.46-7.50 (m, 4H), 7.41 (dd, J=8.1, 1.5 Hz, 1H, ArH), 7.35 (br s, 1H, ArH), 7.18 (d, J=8.3 Hz, 1H, ArH), 4.73 (s, 2H, CH₂), 3.75 (t, J=5.2 Hz, 2H, CH₂), 2.90 (t, J=5.3 Hz, 2H, CH₂), 1.51 (s, 9H) and 1.35 (s, 9H); HRMS (ESI) calcd. for C₂₅H₃₄N₃O₂ (M+H)⁺ 408.2651. found 408.2687.

### tert-butyl N-[7-(4-tert-butylphenyl)-1,2,3,4-tetrahydroisoquinoline-2-carboximidoyl]carbamate 2,2,2-trifluoroacetate (28)

The compound was synthesized as described for 26. A white solid (35 mg, 77%) was obtained. mp 218-219° C. ¹H NMR (400 MHz, CD₃OD) δ 7.50-7.65 (m, 5H, ArH), 7.46 (d, J=1.6 Hz, 1H, ArH), 7.39 (d, J=8.2 Hz, 1H, ArH), 4.70 (s, 2H), 3.70 (t, J=5.5 Hz, 2H, CH₂), 3.00 (t, J=5.5 Hz, 2H, CH₂) and 1.35 (s, 9H); HRMS (ESI) calcd. for C₂₀H₂₆N₃(M+H)⁺ 308.2127. found 308.2131.

### tert-butyl N-[7-(4-tert-butylphenyl)-1,2,3,4-tetrahydroisoquinoline-2-carboximidoyl]carbamate (30)

To a solution of 29 (349 mg, 2.34 mmol) in THF/water (5 mL/1 mL) were added Boc₂O (545 mg, 2.5 mmol) and triethylamine (0.4 mL, 2.8 mmol). The mixture was stirred at room temperature for 16 h, partitioned between ethyl acetate (50) ml and water (50 mL). The organic phase was washed with brine, dried over magnesium sulphate and concentrated in vacuo. The crude product was purified by flash chromatography eluting with 30% ethyl acetate-petrol ether to give a white solid (475 mg, 81% yield). mp 130-131° C. ¹H NMR (400 MHz, CDCl₃) δ 6.98 (d, J=8.2 Hz, 1H, ArH), 6.62-6.65 m, 2H, ArH), 4.51 (s, 2H, CH₂), 3.62 (t, J=6.0 Hz, 2H, CH₂), 2.74 (t, J=5.9 Hz, 2H, CH₂) and 1.49 (s, 9H).

### tert-butyl 7-[(2,3-dichlorophenyl)methoxy]-1,2,3,4-tetrahydroisoquinoline-2-carboxylate (31)

To a solution of 30 (370 mg, 1.48 mmol) in acetone (15 mL) was added 2,3-dicholrobenzyl bromide (437 mg, 1.6 mmol), followed by potassium carbonate (262 mg, 1.9 mmol). The mixture was stirred at ambient temperature overnight, partitioned between ethyl acetate (30) ml and water (30 mL). The organic phase was washed with brine, dried over magnesium sulphate and concentrated in vacuo. Purification of the crude product by flash chromatography eluting with gradient solvent (petrol ether to 25% ethyl acetate-petrol ether) afford the product as clear oil (500 mg, 83%). ¹H NMR (400 MHz, CDCl₃) δ 7.45 (m, 2H, ArH), 7.23 (t, J=7.9 Hz, 1H, ArH), 7.05 (d, J=8.4 Hz, 1H, ArH), 6.75-6.79 (m, 2H, ArH), 5.15 (s, 2H), 4.52 (s, 2H), 3.62 (t, J=5.7 Hz, 2H, CH₂), 2.76 (t, J=5.7 Hz, 2H, CH₂) and 1.48 (s, 9H); HRMS (ESI) calcd. for C₂₁H₂₃Cl₂NNaO₃ (M+Na)⁺ 430.0953. found 430.0970.

### tert-butyl N-[{[(tert-butoxy)carbonyl]imino}({7-[(2,3-dichlorophenyl) methoxy]-1,2,3,4-tetrahydroisoquinolin-2-yl})methyl]carbamate (32)

To a solution of 31 (450 mg, 1.1 mmol) in DCM (63 mL) was added TFA (2 mL). The mixture was shaken at room temperature for 10 h, evaporated in vacuo to give to a yellow residue. The crude product was dissolved in DMF (5 mL) and triethylamine (0.6 mL). S-Methyl-N,N′-bis(tert-butoxycarbonyl)isothiourea (390 mg, 1.32 mmol) was added, followed by HgCl₂ (200 mg, 2.37 mmol). The mixture was stirred at ambient temperature for 16 h, diluted with EtOAc (50 mL), and filtered through Celite. The organic phase was washed with brine, dried over magnesium sulphate and concentrated in vacuo. Purification by flash chromatography eluting with gradient solvent (petrol ether to 40% ethyl acetate-petrol ether) afford the product as a foamy powder solid (340 mg, 56% yield). mp 59-61° C. ¹H NMR (400 MHz, CDCl₃) δ 10.2 (s, 1H, NH), 7.40-7.46 (m, 2H), 7.22 (d, J=8.0 Hz, 1H, ArH)), 7.05 (d, J=8.0 Hz, 1H, ArH)), 6.81 (dd, J=8.0, 1.9 Hz, 1H, ArH)), 6.70 (br s, 1H, ArH), 5.12 (s, 2H), 4.75 (s, 2H), 3.80 (t, J=5.7 Hz, 2H, CH₂), 2.98 (t, J=5.7 Hz, 2H, CH₂) and 1.50 (s, 18H); HRMS (ESI) calcd. for C₂₇H₃₄Cl₂N₃O₅ (M+NH)⁺ 550.1876. found 550.1876.

### 7-[(2,3-dichlorophenyl)methoxy]-1,2,3,4-tetrahydroisoquinoline-2-carboximidamide 2,2,2-trifluoroacetate (33)

The compound was synthesized as described for 26. A white solid (30 mg, 82%) was obtained. ¹H NMR (400 MHz, DMSO-d6) δ 7.70 (d, J=8.0 Hz, 1H, ArH), 7.60 (d, J=8.0 Hz, 1H, ArH), 7.45 (t, J=7.9 Hz, 1H, ArH), 7.25 (d, J=7.7 Hz, 1H, ArH), 6.95 (dd, J=7.8, 1.5 Hz, 1H, ArH), 6.85 (d, J=1.7 Hz, 1H, ArH), 5.20 (s, 2H), 4.51 (s, 2H), 3.50 (t, J=5.2 Hz, 2H, CH₂) and 2.89 (t, J=5.5 Hz, 2H, CH₂); HRMS (ESI) calcd. for C₁₇H₁₈Cl₂N₃O (M+H)⁺ 350.0827. found 350.0821.

### tert-butyl 5-hydroxy-1,2,3,4-tetrahydroisoquinoline-2-carboxylate (35)

The solution of 34 (1.0 g, 90% purity, 6.2 mmol) in AcOH (20 mL) were hydrogenated over PtO₂ (85 mg) at 1 atmosphere for 48 h, filtered through Celite and concentrated in vacuo. The residue was dissolved in acetone (3 mL), and diluted with ethyl ether (3 mL). The precipitate was collected and dried in vacuo. The crude product (700 mg, 4.7 mmol) was suspended in THF/water (10 mL/2 mL). Boc₂O (1.1 g, 5.0 mmol) and triethylamine (1.5 mL, 10 mmol) were added. The mixture was stirred at room temperature for 16 h, partitioned between ethyl acetate (50) ml and water (50 mL). The organic phase was washed with brine, dried over magnesium sulphate and concentrated in vacuo. The crude product was purified by flash chromatography eluting with 30% ethyl acetate-petrol ether to give a white solid (490 mg, 42% yield). mp 156-158° C.; ¹H NMR (400 MHz, CDCl₃) δ 7.03 (d, J=7.8 Hz, 1H, ArH), 6.68 (d, J=7.9 Hz, 1H, ArH), 6.63 (d, J=7.8 Hz, 1H, ArH), 4.55 (s, 2H, CH₂), 3.65 (t, J=6.1 Hz, 2H, CH₂), 2.75 (t, J=6.0 Hz, 2H, CH₂) and 1.48 (s, 9H); HRMS (ESI) calcd. for C₁₄H₁₉NNaO₃ (M+Na)⁺ 272.1263. found 272.1244.

### tert-butyl 5-[(2,3-dichlorophenyl)methoxy]-1,2,3,4-tetrahydroisoquinoline-2-carboxylate (36)

The compound was synthesized as described for 31. A white solid (480 mg, 79%) was obtained. mp 138-139° C.; ¹H NMR (400 MHz, CDCl₃) δ 7.49 (d, J=9.0 Hz, 1H, ArH), 7.44 (d, J=8.7 Hz, 1H, ArH), 7.22-7.26 (m, 2H, ArH), 7.14 (t, J=8.2 Hz, 1H, ArH), 6.74-6.77 (m, 2H, ArH), 5.16 (s, 2H), 4.58 (s, 2H), 3.67 (t, J=5.9 Hz, 2H, CH₂), 2.86 (t, J=5.8 Hz, 2H, CH₂) and 1.49 (s, 9H); HRMS (ESI) calcd. for C₂₁H₂₃Cl₂NNaO₃ (M+Na)⁺ 430.0953. found 430.0917.

### tert-butyl N-[{[(tert-butoxy)carbonyl]amino}({5-[(2,3-dichlorophenyl)methoxy]-1,2,3,4-tetrahydroisoquinolin-2-yl})methylidene]carbamate (37)

The compound was synthesized as described for 32. A white solid (280 mg, 59%) was obtained. mp 129-130° C. ¹H NMR (400 MHz, CDCl₃) δ 7.52 (d, J=8.3 Hz, 1H, ArH), 7.46 (d, J=8.5 Hz, 1H, ArH), 7.31-7.34 (m, 2H, ArH), 7.18 (t, J=8.3 Hz, 1H, ArH)), 6.79-6.82 (m, 2H, ArH), 5.21 (s, 2H), 4.62 (s, 2H), 3.71 (t, J=5.5 Hz, 2H, CH₂), 2.91 (t, J=5.5 Hz, 2H, CH₂), 1.51 (s, 9H); and 1.47 (s, 9H); HRMS (ESI) calcd. for C₂₇H₃₄Cl₂N₃O₅ (M+NH)⁺ 550.1876. found 550.1882.

### 5-[(2,3-dichlorophenyl)methoxy]-1,2,3,4-tetrahydroisoquinoline-2-carboximidamide hydrochloride (38)

The compound in TFA salt form (100 mg) was synthesized as described for 26. The TFA salt was converted to hydrochloride 38 using 0.5 M HCl—CH3OH solution. ¹H NMR (400 MHz, DMSO-d6) δ 7.73 (d, J=8.5 Hz, 1H, ArH), 7.65 (d, J=8.3 Hz, 1H, ArH), 7.49 (m, 1H, ArH), 7.28 (t, J=8.0 Hz, 1H, ArH), 7.05 (d, J=7.9 Hz, 1H, ArH), 6.82 (d, J=7.8 Hz, 1H, ArH), 5.35 (s, 2H), 4.60 (s, 2H), 3.52 (t, J=5.2 Hz, 2H, CH₂) and 2.85 (t, J=5.5 Hz, 2H, CH₂); HRMS (ESI) calcd. for C₁₇H₁₈Cl₂N₃O (M+H)⁺ 350.0827. found 350.0899.

### General Procedure: Guanylation of O-benzylhydroxylamine (39a-39c) and 1-phenylpiperazine (42a-42b)

To a solution of the substituted amine (1.5 mmol) in DMF (5 mL) was added S-methyl-N,N′-bis(tert-butoxycarbonyl)isothiourea (285 mg, 0.98 mmol), followed by triethylamine (0.6 mL). The mixture was stirred at ambient temperature overnight, partitioned between ethyl acetate (100) ml and brine (50 mL). The organic phase was washed with brine, dried over magnesium sulphate and concentrated in vacuo. The crude product was purified by flash chromatography eluting with gradient solvent (petrol ether to 45% ethyl acetate-petrol ether) to afford the product (40a-40c) and (43a-43b).

### tert-butyl N-[{[(tert-butoxy)carbonyl]amino}({[(3-phenylphenyl)methoxy]amino}) methylidene]carbamate (40a)

A foamy powder (190 mg, 69%) was obtained. ¹H NMR (400 MHz, CDCl₃) δ 9.16 (s, 1H, NH), 7.73 (s, 1H, NH), 7.56-7.68 (m, 3H, ArH), 7.33-7.46 (m, 5H, ArH), 5.13 (s, 2H), 1.49 (s, 9H) and 1.46 (s, 9H).

### tert-butyl N-[{[(tert-butoxy)carbonyl]amino}({[(3-phenoxyphenyl)methoxy]amino})methylidene]carbamate (40b)

A foamy powder (185 mg, 65%) was obtained. ¹H NMR (400 MHz, CDCl₃) δ 7.82 (d, J=8.2 Hz, 1H, ArH), 7.81 (d, J=8.1 Hz, 1H, ArH), 7.76 (s, 1H, NH), 7.73 (m, 3H, ArH), 7.59 (dt, J=8.1, 1.5 Hz, 1H, ArH), 7.41-7.55 (m, 4H, ArH), 5.28 (s, 2H) and 1.36 (s, 18H).

### tert-butyl N-[{[(tert-butoxy)carbonyl]amino}({[3-(4-tert-butylphenyl)phenyl]methoxy}amino)methylidene]carbamate (40c)

A foamy powder (200 mg, 80%) was obtained. ¹H NMR (400 MHz, CDCl₃) δ 9.18 (s, 1H, NH), 7.65 (br s, 1H), 7.52-7.58 (m, 3H, ArH), 7.38-7.45 (m, 4H, ArH), 5.15 (s, 2H), 1.50 (s, 9H), 1.49 (s, 9H) and 1.36 (s, 9H).

### tert-butyl (((tert-butoxycarbonyl)amino)(4-(4-chlorophenyl)piperazin-1-yl)methylene)carbamate (43a)

A foamy powder (188 mg, 80%) was obtained. ¹H NMR (400 MHz, CDCl₃) δ 10.3 (s, 1H, NH), 7.20 (dt, J=8.6, 1.9 Hz, 2H, ArH), 6.82 (dt, J=8.6, 2.1 Hz, 2H, ArH), 3.75 (br s, 4H), 3.22 (t, J=5.2 Hz, 4H) and 1.50 (s, 18H); HRMS (ESI) calcd. for C₂₁H₃₁ClN₄NaO₄ (M+H)⁺ 461.1932. found 461.18.74.

### tert-butyl (((tert-butoxycarbonyl)amino)(4-(3-methoxyphenyl)piperazin-1-yl)methylene)carbamate (43b)

A foamy powder (141 mg, 75%) was obtained. ¹H NMR (400 MHz, CDCl₃) δ 10.2 (s, 1H, NH), 7.18 (t, J=8.8 Hz, 1H, ArH), 6.53 (d, J=8.6 Hz, 1H, ArH), 6.43 (m, 2H, ArH), 3.80 (s, 3H), 3.75 (br s, 4H), 3.25 (t, J=5.2 Hz, 4H) and 1.50 (s, 18H); HRMS (ESI) calcd. for C₂₂H₃₄N₄NaO₅ (M+H)⁺ 457.2427. found 457.2376.

### General Procedure: Synthesis of the Guanidino Derivatives (41a-41c and 44a-44b)

To a solution of the substituted N,N′-di-Boc-guanidino derivative (40a-40c) or (44a-44b) (0.3 mmol) in DCM (2 mL) was added trifluoroacetic acid (1 mL). The mixture was shaken at ambient temperature overnight, evaporated to dryness. Ethyl ether (1 mL) was added, and the precipitate was collected and washed with ether, dried in vacuo to give the target compound as a white or off-white solid.

### 1-[(3-phenylphenyl)methoxy]guanidine 2,2,2-trifluoroacetate (41a)

An off-white solid (65 mg, 92%) was obtained. ¹H NMR (400 MHz, DMSO-d6) δ 11.2 (s, 1H), 7.83 (br s, 1H), 7.70-7.76 (m, 4H, ArH), 7.72 (t, J=1.9 Hz, 1H, ArH), 7.43-7.57 (m, 4H, ArH) and 5.00 (s, 2H); HRMS (ESI) calcd. for C₁₄H₁₆N₃O (M+H)⁺ 242.1293. found 242.1282.

### 1-[(3-phenoxyphenyl)methoxy]guanidine 2,2,2-trifluoroacetate (41b)

A white solid (95 mg, 97%) was obtained. ¹H NMR (400 MHz, CDCl₃) δ 10.9 (s, 1H), 7.30-7.35 (m, 3H, ArH), 7.12 (td, J=8.9, 2.1 Hz, 1H, ArH), 7.05 (dt, J=8.5, 1.9 Hz, 1H, ArH), 6.96-7.05 (m, 4H, ArH) and 4.76 (s, 2H); HRMS (ESI) calcd. for C₁₄H₁₅N₃NaO₂ (M+Na)⁺ 280.1062. found 280.1066.

### 1-{[3-(4-tert-butylphenyl)phenyl]methoxy}guanidine 2,2,2-trifluoroacetate (41c)

A white solid (75 mg, 77%) was obtained. ¹H NMR (400 MHz, CD₃OD) δ 7.88 (br s, 1H), 7.72 (dt, J=8.8, 2.0 Hz, 1H, ArH), 7.63-7.66 (m, 2H, ArH), 7.52-7.56 (m, 3H, ArH), 7.46 (dt, J=8.9, 1.7 Hz, 1H, ArH), 5.12 (s, 2H) and 1.43 (s, 9H); HRMS (ESI) calcd. for C₁₈H₂₄N₃O (M+H)⁺ 298.1919. found 298.1938.

### 4-(4-chlorophenyl)piperazine-1-carboximidamide bis-(2,2,2-trifluoroacetate) (44a)

An off-white solid (70 mg, 86%) was obtained. ¹H NMR (400 MHz, CD₃OD) δ 7.31 (dt, J=8.9, 2.3 Hz, 2H, ArH), 7.63 (dt, J=8.8, 2.3 Hz, 2H, ArH), 3.71 (t, J=5.2 Hz, 4H) and 3.33 (t, J=5.2 Hz, 4H); HRMS (ESI) calcd. for C₁₁H₁₆ClN₄ (M+H)⁺ 239.1063. found 239.1052.

### 4-(3-methoxyphenyl)piperazine-1-carboximidamide bis-(2,2,2-trifluoroacetate) (44b)

An off-white solid (65 mg, 82%) was obtained. ¹H NMR (400 MHz, CD₃OD) δ 7.19 (t, J=8.8 Hz, 1H, ArH), 6.61 (dt, J=8.3, 1.9 Hz, 1H, ArH), 6.55 (t, J=1.7 Hz, 1H, ArH), 3.80 (s, 3H), 3.65 (t, J=5.3 Hz, 4H) and 3.28 (t, J=5.3 Hz, 4H); HRMS (ESI) calcd. for C₁₂H₁₉N₄O (M+H)⁺ 235.1558. found 235.1547.

### tert-butyl N-[{[3-(benzyloxy)phenyl]formamido}({[(tert-butoxy)carbonyl]imino})methyl]carbamate (46)

To a solution of 3-(benzyloxy)benzoyl chloride (45, 1.0 g, 4.06 mmol) in DCM (40 mL) was added 1,3-bis-Boc-guanidine (1.05 g, 4.0 mmol), followed by triethylamine (1.7 mL). The mixture was stirred at ambient temperature overnight, partitioned between DCM (50) ml and 5% citric acid (50 mL). The organic phase was washed with brine, dried over magnesium sulphate and concentrated in vacuo. The crude product was purified by flash chromatography eluting with gradient solvent (petrol ether to 30% ethyl acetate-petrol ether) to afford the product as white solid (1.2 g, 64% yield). ¹H NMR (400 MHz, CDCl₃) δ 7.85 (br s, 2H), 7.46 (d, J=8.8 Hz, 2H, ArH), 7.35-7.40 (m, 5H), 7.18 (d, J=8.9 Hz, 2H, ArH), 5.12 (s, 2H) and 1.52 (s, 18H); HRMS (ESI) calcd. for C₂₅H₃₂N₃O₆ (M+H)⁺ 470.2291. found 470.2279.

### 3-(benzyloxy)-N-carbamimidoylbenzamide 2,2,2-trifluoroacetate (47)

The compound was synthesized as described for compound 26. A white solid (110 mg, 98% yield) was obtained. ¹H NMR (400 MHz, DMSO-d6) δ 8.68 (br s, 2H), 8.50 (br s, 2H), 7.62 (d, J=8.9 Hz, 2H, ArH), 7.53 (d, J=8.9 Hz, 2H, ArH), 7.38-7.46 (m, 5H) and 5.26 (s, 2H); HRMS (ESI) calcd. for C₁₅H₁₆N₃O₂ (M+H)⁺ 270.1243. found 270.1308.

### General Procedure: Synthesis of 3-benzyloxybenzaldehyde Derivatives (48b-f; 50a-l)

The 3-hydroxybenzaldehyde derivative (2 mmol) and potassium carbonate (4 mmol) were placed in a round-bottom flask. N,N-dimethylformamide (3 mL) was added. Then the corresponding benzyl halide (2.2 mmol) was added and the reaction mixture was stirred for 18 h at room temperature. Water (125 mL) and brine (25 mL) were added and the mixture was extracted with diethyl ether (2×100 mL) or dichloromethane (for 50i; 2×100 mL). The combined organic layers were dried (NaCl), filtered and concentrated in vacuo. Crystallisation from diethyl ether or pentane/diethyl ether gave the corresponding aldehyde derivatives 48b, 50a-l. Aldehyde derivatives 48c-f were purified by column chromatography.

### 3-(4-Chlorobenzyloxy)-benzaldehyde (48b)

430 mg, 87%, white solid, m.p. 52-54° C., ¹H NMR (400 MHz, CDCl₃): δ5.08 (2H, s, CH₂), 7.23 (1H, dt, J 7.6, 2.0, CH), 7.34-7.40 (4H, m, 4×CH), 7.43-7.51 (3H, m, 3×CH), 9.97 (1H, s, CH═O). HRMS (ESI) calcd. for C₁₄H₁₂ClO₂⁺ (M+H)⁺ 247.0520. found 247.0531.

### 3-(4-(Trifluoromethyl)benzyloxy)-benzaldehyde (48c)

Purification by flash column chromatography (pentane to pentane/ethyl acetate 9:1) gave 49d (501 mg, 89%, colourless oil). ¹H NMR (400 MHz, CDCl₃): δ5.18 (2H, s, CH₂), 7.23-7.28 (1H, m, CH), 7.44-7.52 (3H, m, 3×CH), 7.56 (2H, d, J 8.0, 2×CH), 7.66 (2H, d, J 8.0, 2×CH), 9.97 (1H, d, J 1.2, CH═O). HRMS (ESI) calcd. for C₁₅H₁₂F₃O₂ (M+H)⁺ 281.0784. found 281.0789.

### 3-(3-Chlorobenzyloxy)-benzaldehyde (48d)

Purification by flash column chromatography (pentane to pentane/ethyl acetate 9:1) gave 48d (424 mg, 86%, colourless oil). ¹H NMR (400 MHz, CDCl₃): δ5.09 (2H, s, CH₂), 7.22-7.26 (1H, m, CH), 7.29-7.33 (3H, m, 3×CH), 7.44-7.51 (4H, m, 4×CH), 9.97 (1H, s, CH═O). HRMS (ESI) calcd. for C₁₄H₁₂ClO₂⁺ (M+H)⁺ 247.0520. found 247.0525.

### 3-(3-(Trifluoromethyl)benzyloxy)-benzaldehyde (48e)

Purification by flash column chromatography (pentane to pentane/ethyl acetate 9:1) gave 48e (478 mg, 85%, colourless oil). ¹H NMR (400 MHz, CDCl₃): δ5.17 (2H, s, CH₂), 7.24-7.28 (1H, m, CH), 7.45-7.51 (3H, m, 3×CH), 7.53 (1H, d, J 8.0, CH), 7.61 (1H, d, J 8.8, CH), 7.63 (1H, d, J 8.0, CH), 7.72 (1H, s, CH), 9.98 (1H, s, CH═O). HRMS (ESI) calcd. for C₁₅H₁₂F₃O₂ (M+H)⁺ 281.0784. found 281.0790.

### 3-((3,4-dichlorobenzyl)oxy)-4-methoxybenzaldehyde (48f)

Purification by flash column chromatography (DCM to DCM/ethyl acetate 9:1) gave 48f (436 mg, 87%). m.p. 117-119° C. ¹H NMR (400 MHz, CDCl₃): δ3.98 (3H, s, OCH₃), 5.28 (2H, s, CH₂), 7.08 (1H, d, J 8.3, ArH), 7.23 (1H, d, J 8.1, ArH), 7.43 (2H, d, J 8.2, ArH), 7.52 (2H, dd, J 8.0, 1.1, ArH), 9.88 (1H, s, CHO). HRMS (ESI) calcd. for C₁₅H₁₃Cl₂O₃(M+H)⁺ 311.0242. found 311.0237.

### 2-Chloro-3-(2-chloro-3-methoxybenzyloxy)-benzaldehyde (50a)

597 mg, 96%, white solid, m.p. 124-126° C., ¹H NMR (400 MHz, CDCl₃): δ3.92 (3H, s, OCH₃), 5.28 (2H, s, CH₂), 6.92 (1H, dd, J 7.0, 2.6, CH), 7.19 (1H, dd, J 8.0, 1.2, CH), 7.24-7.33 (3H, m, 3×CH), 7.54 (1H, dd, J 7.8, 1.4, CH), 10.53 (1H, d, J 0.4, CH═O). HRMS (ESI) calcd. for C₁₅H₁₃Cl₂O₃⁺ (M+H)⁺ 311.0236. found 311.0232.

### 2-Chloro-3-benzyloxybenzaldehyde (50b)

425 mg, 86%, white solid, m.p. 103-105° C., ¹H NMR (400 MHz, CDCl₃): δ5.20 (2H, s, CH₂), 7.19 (1H, dd, J 8.0, 1.2, CH), 7.29 (1H, t, J 7.8, CH), 7.35 (1H, d, J 6.8, CH), 7.41 (2H, t, J 7.4, 2×CH), 7.47 (2H, d, J 7.2, 2×CH), 7.53 (1H, dd, J 7.8, 1.4, CH), 10.54 (1H, s, CH═O). HRMS (ESI) calcd. for C₁₄H₁₂ClO₂⁺ (M+H)⁺ 247.0520. found 247.0528.

### 2-Chloro-3-(4-chlorobenzyloxy)-benzaldehyde (50c)

522 mg, 93%, white solid, m.p. 94-96° C., ¹H NMR (400 MHz, CDCl₃): δ5.15 (2H, s, CH₂), 7.16 (1H, dd, J 8.0, 1.6, CH), 7.30 (1H, t, J 8.0, CH), 7.35-7.43 (4H, m, 4×CH), 7.54 (1H, dd, J 7.8, 1.4, CH), 10.53 (1H, d, J 0.8, CH═O). HRMS (ESI) calcd. for C₁₄H₁₁Cl₂O₂⁺ (M+H)⁺ 281.0131. found 281.0127.

### 2-Chloro-3-(2,3-dichlorobenzyloxy)-benzaldehyde (50d)

600 mg, 95%, white solid, m.p. 150-152° C., ¹H NMR (400 MHz, CDCl₃): δ5.27 (2H, s, CH₂), 7.20 (1H, d, J 8.0, CH), 7.28 (1H, t, J 8.0, CH), 7.34 (1H, t, J 8.0, CH), 7.46 (1H, d, J 8.0, CH), 7.57 (1H, d, J 8.0, CH), 7.61 (1H, d, J 8.0, CH), 10.54 (1H, s, CH═O). HRMS (ESI) calcd. for C₁₄H₁₀Cl₃O₂⁺ (M+H)⁺ 314.9741. found 314.9748.

### 2-Chloro-3-(2-chloro-3-(trifluoromethyl)benzyloxy)-benzaldehyde (50e)

632 mg, 90%, white solid, m.p. 128-131° C., ¹H NMR (400 MHz, CDCl₃): δ5.31 (2H, s, CH₂), 7.23 (1H, dd, J 8.2, 1.4, CH), 7.36 (1H, t, J 8.0, CH), 7.47 (1H, t, J 7.8, CH), 7.59 (1H, dd, J 7.8, 1.4, CH), 7.71 (1H, d, J 8.0, CH), 7.94 (1H, d, J 7.6, CH), 10.55 (1H, d, J 0.8, CH═O). HRMS (ESI) calcd. for C₁₅H₁₀Cl₂F₃O₂⁺ (M+H)⁺ 349.0004. found 349.0011.

### 2-Chloro-3-(2,4-dichlorobenzyloxy)-benzaldehyde (50f)

580 mg, 92%, white solid, m.p. 115-116° C., ¹H NMR (400 MHz, CDCl₃): δ5.22 (2H, s, CH₂), 7.20 (1H, dd, J 8.2, 1.4, CH), 7.30-7.36 (2H, m, 2×CH), 7.43 (1H, d, J 2.4, CH), 7.57 (1H, dd, J 7.8, 1.4, CH), 7.62 (1H, d, J 8.4, CH), 10.54 (1H, d, J 0.8, CH═O). HRMS (ESI) calcd. for C₁₄H₁₀Cl₃O₂⁺ (M+H)⁺ 314.9741. found 314.9748.

### 2-Chloro-3-(2,5-dichlorobenzyloxy)-benzaldehyde (50g)

535 mg, 85%, white solid, m.p. 132-134° C., ¹H NMR (400 MHz, CDCl₃): δ5.22 (2H, s, CH₂), 7.21 (1H, dd, J 8.4, 1.2, CH), 7.27 (1H, dd, J 8.4, 2.4, CH), 7.34 (1H, d, J 8.8, CH), 7.35 (1H, t, J 8.0, CH), 7.58 (1H, dd, J 8.0, 1.6, CH), 7.69 (1H, d, J 2.4, CH), 10.55 (1H, s, CH═O). HRMS (ESI) calcd. for C₁₄H₁₀Cl₃O₂⁺ (M+H)⁺ 314.9741. found 314.9745.

### 2-Chloro-3-(3,4-dichlorobenzyloxy)-benzaldehyde (50h)

610 mg, 97%, white solid, m.p. 140-142° C., ¹H NMR (400 MHz, CDCl₃): δ5.13 (2H, s, CH₂), 7.15 (1H, dd, J 8.2, 1.4, CH), 7.29-7.34 (2H, m, 2×CH), 7.48 (1H, d, J 8.4, CH), 7.56 (1H, dd, J 8.0, 1.2, CH), 7.58 (1H, s, CH), 10.53 (1H, s, CH═O). HRMS (ESI) calcd. for C₁₄H₁₀Cl₃O₂⁺ (M+H)⁺ 314.9741. found 314.9744.

### 2-Chloro-3-(2,3,5-trichlorobenzyloxy)-benzaldehyde (50i)

610 mg, 87%, white solid, m.p. 158-161° C., ¹H NMR (400 MHz, CDCl₃): δ5.22 (2H, s, CH₂), 7.20 (1H, dd, J 8.2, 1.4, CH), 7.36 (1H, t, J 8.0, CH), 7.48 (1H, d, J 2.4, CH), 7.60 (1H, dd, J 8.0, 1.2, CH), 7.64 (1H, d, J 2.4, CH), 10.55 (1H, d, J 0.4, CH═O). HRMS (ESI) calcd. for C₁₄H₉Cl₄O₂⁺ (M+H)⁺ 348.9351. found 348.9365.

### 2-Chloro-3-(3-(trifluoromethyl)benzyloxy)-benzaldehyde (50j)

617 mg, 98%, white solid, m.p. 83-85° C., ¹H NMR (400 MHz, CDCl₃): δ5.23 (2H, s, CH₂), 7.19 (1H, dd, J 8.0, 1.2, CH), 7.33 (1H, t, J 8.0, CH), 7.54 (1H, t, J 7.8, CH), 7.57 (1H, dd, J 7.8, 1.4, CH), 7.62 (1H, d, J 8.0, CH), 7.69 (1H, d, J 7.6, CH), 7.74 (1H, s, CH), 10.54 (1H, d, J 0.4, CH═O). HRMS (ESI) calcd. for C₁₅H₁₁ClF₃O₂⁺ (M+H)⁺ 315.0394. found 315.0401.

### 2-Chloro-3-(4-(trifluoromethyl)benzyloxy)-benzaldehyde (50k)

605 mg, 96%, white solid, m.p. 82-84° C., ¹H NMR (400 MHz, CDCl₃): δ5.25 (2H, s, CH₂), 7.18 (1H, dd, J 7.8, 1.4, CH), 7.32 (1H, dt, J 8.0, 0.8, CH), 7.56 (1H, dd, J 7.8, 1.4, CH), 7.60 (2H, d, J 8.0, 2×CH), 7.67 (2H, d, J 7.6, 2×CH), 10.54 (1H, s, CH═O). HRMS (ESI) calcd. for C₁₅H₁₁ClF₃O₂(M+H)⁺ 315.0394. found 315.0398.

### 2-Chloro-3-(3-chlorobenzyloxy)-benzaldehyde (50l)

418 mg, 74%, white solid). ¹H NMR (400 MHz, CDCl₃): δ5.16 (2H, s, CH₂), 7.17 (1H, dd, J 8.2, 1.4, CH), 7.31 (1H, t, J 8.0, CH), 7.31-7.37 (3H, m, 3×CH), 7.46-7.48 (1H, m, CH), 7.56 (1H, dd, J 7.8, 1.4, CH), 10.54 (1H, d, J 0.8, CH═O). HRMS (ESI) calcd. for C₁₄H₁₁Cl₂O₂⁺ (M+H)⁺ 281.0131. found 281.0139.

### 1-[({3-[(2,3-dichlorophenyl)methoxy]phenyl}methylidene)amino]guanidine acetate (49a)

The mixture of 3-[(2,3-dichlorophenyl)methoxy]benzaldehyde (5, 228 mg, 0.81 mmol) and aminoguanidine hydrogen carbonate (110 mg, 0.81 mmol) in methanol-AcOH (3 mL/0.2 mL) was refluxed under nitrogen for 4 h, cooled to room temperature and concentrated in vacuo. DCM (1 mL) was added, and the precipitate was collected, washed with DCM and dried in vacuo to give a white solid (170 mg, 53% yield). mp 159-160° C.; ¹H NMR (400 MHz, DMSO-d6) δ 8.08 (s, 1H), 7.66-7.72 (m, 2H), 7.58 (s, 1H), 7.48 (t, J=7.9 Hz, 1H, ArH), 7.53-7.55 (m, 2H), 7.03 (d, J=8.9 Hz, 1H, ArH), 7.07 (br s, 4H), 5.29 (s, 2H) and 1.87 (s, 3H); HRMS (ESI) calcd. for C₁₅H₁₅Cl₂N₄O (M+H)⁺ 337.0623. found 337.0693.

### 1-{[(3-{[2-chloro-3-(trifluoromethyl)phenyl]methoxy}phenyl)methylidene]amino} guanidine acetate (49b)

The mixture of 48a (320 mg, 1.02 mmol) and aminoguanidine hydrogen carbonate (138 mg, 1.02 mmol) in methanol-AcOH (4 mL/0.2 mL) was refluxed under nitrogen for 6 h, cooled to room temperature and concentrated in vacuo. EtOAc (3 mL) was added, and the precipitate was collected, washed with EtOAc and dried in vacuo to give a white solid (283 mg, 75% yield). mp 190-192° C.; ¹H NMR (400 MHz, DMSO-d6) δ 7.99 (s, 1H), 7.91 (d, J=7.8 Hz, 1H, ArH), 7.86 (d, J=8.0 Hz, 1H, ArH), 7.58 (t, J=7.8 Hz, 1H, ArH), 7.50 (s, 1H), 7.26-7.33 (m, 2H), 7.02 (dd, J=7.8, 1.9 Hz, 1H, ArH), 6.95 (br s, 4H), 5.25 (s, 2H) and 1.82 (s, 3H); HRMS (ESI) calcd. for C16H15ClF3N4O (M+H)⁺ 371.0886. found 371.0895.

### General procedure: Synthesis of (E)-amino(2-(3-(benzyloxy)benzylidene) hydrazinyl)methaniminium chloride derivatives (49c-g, 51a-m)

3-benzyloxybenzaldehyde derivatives (48b-f, 50a-l) (0.2 mmol) and N-aminoguanidine hydrocarbonate (0.205 mmol) were placed in a 50 mL round-bottom flask. HCl (0.5M in methanol, 2.0 mL; for 51j: 1.8 mL methanol and 0.2 mL acetic acid) was added and the reaction mixture was stirred at 80° C. for 0.5 h (for 51j: 80° C. for 2 h) and then evaporated to dryness. Crystallisation from diethyl ether (˜5-6 mL) with a very small portion of methanol (˜0.3-0.5 mL) gave the corresponding N-aminoguanidinium chloride (acetate) salts 49c-g, 51a-m.

### (E)-Amino(2-(3-((4-chlorobenzyl)oxy)benzylidene)hydrazinyl)methaniminium chloride (49c)

54 mg, 79%, white solid, m.p. 191-193° C., ¹H NMR (400 MHz, DMSO-d₆): δ5.21 (2H, s, CH₂), 7.11-7.18 (1H, m, CH), 7.38-7.45 (2H, m, CH), 7.52 (2H, d, J 8.4, 2×CH), 7.56 (2H, d, J 8.4, 2×CH), 7.67 (1H, s, CH), 7.86 (4H, s, br, 2×NH₂), 8.20 (1H, s, CH═N), 11.97 (1H, s, N—NH). HRMS (ESI) calcd. for C₁₅H₁₆ClN₄O⁺ (M)⁺ 303.1007. found 303.1013.

### (E)-Amino(2-(3-((4-(trifluoromethyl)benzyl)oxy)benzylidene) hydrazinyl) methaniminium chloride (49d)

58 mg, 77%, white solid, m.p. 176-179° C., ¹H NMR (400 MHz, DMSO-d₆): δ5.33 (2H, s, CH₂), 7.14-7.20 (1H, m, CH), 7.40-7.47 (2H, m, 2×CH), 7.67 (1H, s, CH), 7.75 (2H, d, J 8.0, 2×CH), 7.83 (2H, d, J 8.4, 2×CH), 7.85 (4H, s, br, 2×NH₂), 8.20 (1H, s, CH═N), 11.96 (1H, s, N—NH). HRMS (ESI) calcd. for C₁₆H₁₆F₃N₄O⁺ (M)⁺ 337.1271. found 337.1278.

### (E)-Amino(2-(3-((3-chlorobenzyl)oxy)benzylidene)hydrazinyl)methaniminium chloride (49e)

69 mg, >99%, beige glass, ¹H NMR (400 MHz, DMSO-d₆): δ5.22 (2H, s, CH₂), 7.12-7.18 (1H, m, CH), 7.42 (2H, d, J 6.0, 2×CH), 7.45 (1H, dd, J 5.0, 2.0, CH), 7.49 (2H, d, J 6.0, CH), 7.59 (1H, s, CH), 7.66 (1H, d, J 2.8, CH), 7.84 (4H, s, br, 2×NH₂), 8.21 (1H, s, CH═N), 11.97 (1H, s, N—NH). HRMS (ESI) calcd. for C₁₅H₁₆ClN₄O⁺ (M)⁺ 303.1007. found 303.1015.

### (E)-Amino(2-(3-((4-(trifluoromethyl)benzyl)oxy)benzylidene)hydrazinyl) methaniminium chloride (49f)

68 mg, 92%, pale yellow glass, ¹H NMR (400 MHz, DMSO-d₆): δ5.31 (2H, s, CH₂), 7.15-7.21 (1H, m, CH), 7.41-7.45 (2H, m, 2×CH), 7.69 (1H, d, J 2.8, CH), 7.71 (1H, d, J 8.4, CH), 7.76 (1H, d, J 8.0, CH), 7.84 (1H, d, J 7.6, CH), 7.86 (4H, s, br, 2×NH₂), 7.89 (1H, s, CH), 8.22 (1H, s, CH═N), 11.97 (1H, s, N—NH). HRMS (ESI) calcd. for C₁₆H₁₆F₃N₄O⁺ (M)⁺ 337.1271. found 337.1282.

### (E)-2-(3-((3,4-dichlorobenzyl)oxy)-4-methoxybenzylidene)hydrazine-1-carboximidamide acetate (49g)

278 mg (80%) white solid was obtained. ¹H NMR (400 MHz, DMSO-d₆): δ1.89 (3H, s), 3.85 (3H, s), 5.30 (2H, s, CH₂), 6.98 (3H, br s), 7.07 (1H, d, J 8.1, ArH), 7.28 (1H, d, J 8.2, ArH), 7.50 (1H, t, J 7.9, ArH), 7.65-7.75 (3H, m), 8.19 (1H, s, CH═N). HRMS (ESI) calcd. for C₁₆H₁₇Cl₂N4O₂ (M+H)⁺ 367.0729. found 367.0701.

### (E)-Amino(2-(2-chloro-3-((2-chloro-3-methoxybenzyl)oxy)benzylidene) hydrazinyl)methaniminium chloride (51a)

68 mg, 84%, white solid, m.p. 258-260° C., ¹H NMR (400 MHz, DMSO-d₆): δ5.33 (2H, s, CH₂), 7.23 (1H, dd, J 8.0, 1.2, CH), 7.28 (1H, dd, J 7.6, 1.2, CH), 7.37 (1H, dd, J 8.0, 1.6, CH), 7.42 (1H, d, J 8.0, CH), 7.43 (1H, t, J 7.8, CH), 7.93 (4H, s, br, 2×NH₂), 7.96 (1H, dd, J 7.8, 1.4, CH), 8.65 (1H, s, CH═N), 12.26 (1H, s, N—NH). HRMS (ESI) calcd. for C₁₆H₁₇Cl₂N₄O₂⁺ (M)⁺ 367.0723. found 367.0734.

### (E)-Amino(2-(3-(benzyloxy)-2-chlorobenzylidene)hydrazinyl)methaniminium chloride (51b)

56 mg, 82%, white solid, m.p. 203-205° C., ¹H NMR (400 MHz, DMSO-d₆): δ5.30 (2H, s, CH₂), 7.37 (1H, dd, J 8.4, 2.0, CH), 7.39-7.44 (2H, m, 2×CH), 7.47 (2H, t, J 7.4, 2×CH), 7.53 (1H, s, CH), 7.55 (1H, d, J 1.6, CH), 7.92 (4H, s, br, 2×NH₂), 7.94 (1H, dd, J 7.6, 1.6, CH), 8.65 (1H, s, CH═N), 12.27 (1H, s, N—NH). HRMS (ESI) calcd. for C₁₅H₁₆ClN₄O⁺ (M)⁺ 303.1007. found 303.1012.

### (E)-Amino(2-(2-chloro-3-((4-chlorobenzyl)oxy)benzylidene)hydrazinyl) methaniminium chloride (51c)

63 mg, 84%, white solid, m.p. 237-239° C., ¹H NMR (400 MHz, DMSO-d₆): δ5.30 (2H, s, CH₂), 7.35 (1H, dd, J 8.0, 1.2, CH), 7.42 (1H, t, J 8.0, CH), 7.51-7.59 (4H, m, CH), 7.93 (4H, s, br, 2×NH₂), 7.95 (1H, dd, J 7.6, 1.6, CH), 8.65 (1H, s, CH═N), 12.32 (1H, s, N—NH). HRMS (ESI) calcd. for C₁₅H₁₅Cl₂N₄O⁺ (M)⁺ 337.0617. found 337.0625.

### (E)-Amino(2-(2-chloro-3-((2,3-dichlorobenzyl)oxy)benzylidene)hydrazinyl) methaniminium chloride (51d)

71 mg, 87%, white solid, m.p. 250-253° C., ¹H NMR (400 MHz, DMSO-d₆): δ5.38 (2H, s, CH₂), 7.40 (1H, dd, J 8.4, 1.6, CH), 7.46 (1H, t, J 7.6, CH), 7.51 (1H, t, J 8.0, CH), 7.70 (1H, dd, J 7.8, 1.4, CH), 7.73 (1H, dd, J 8.0, 1.2, CH), 7.95 (4H, s, br, 2×NH₂), 7.98 (1H, dd, J 7.6, 1.6, CH), 8.65 (1H, s, CH═N), 12.30 (1H, s, N—NH). HRMS (ESI) calcd. for C₁₅H₁₄Cl₃N₄O⁺ (M)⁺ 371.0228. found 371.0235.

### (E)-Amino(2-(2-chloro-3-((2-chloro-3-(trifluoromethyl)benzyl)oxy)benzylidene) hydrazinyl)methaniminium chloride (51e)

73 mg, 82%, white solid, m.p. 274-277° C., ¹H NMR (400 MHz, DMSO-d₆): δ5.44 (2H, s, CH₂), 7.42-7.48 (2H, m, CH), 7.71 (1H, t, J 7.8, CH), 7.95 (1H, d, J 8.0, CH), 7.97 (4H, s, br, 2×NH₂), 7.99 (1H, dd, J 7.0, 1.4, CH), 8.04 (1H, d, J 7.6, CH), 8.66 (1H, s, CH═N), 12.31 (1H, s, N—NH). ¹³C NMR (100 MHz, DMSO-d₆): δ 67.6 (CH₂), 115.5 (CH), 119.8 (CH), 122.3, 127.6 (CH), 127.8 (CH), 127.9 (CH), 132.0, 133.5 (CH), 136.7, 142.9 (CH), 153.5, 155.2. HRMS (ESI) calcd. for C₁₆H₁₄Cl₂F₃N₄O⁺ (M)⁺ 405.0491. found 405.0498.

### (E)-Amino(2-(2-chloro-3-((2,4-dichlorobenzyl)oxy)benzylidene)hydrazinyl) methaniminium chloride (51f)

73 mg, 89%, white solid, m.p. 268-270° C., ¹H NMR (400 MHz, DMSO-d₆): δ5.32 (2H, s, CH₂), 7.40 (1H, dd, J 8.4, 1.6, CH), 7.44 (1H, t, J 8.0, CH), 7.58 (1H, dd, J 8.4, 2.0, CH), 7.73 (1H, d, J 8.4, CH), 7.95 (4H, s, br, 2×NH₂), 7.98 (1H, dd, J 7.6, 1.6, CH), 8.65 (1H, s, CH═N), 12.34 (1H, s, N—NH). HRMS (ESI) calcd. for C₁₅H₁₄Cl₃N₄O⁺ (M)⁺ 371.0228. found 371.0239.

### (E)-Amino(2-(2-chloro-3-((2,5-dichlorobenzyl)oxy)benzylidene)hydrazinyl) methaniminium chloride (51g)

72 mg, 88%, white solid, m.p. 242-244° C., ¹H NMR (400 MHz, DMSO-d₆): δ5.33 (2H, s, CH₂), 7.42 (1H, dd, J 7.4, 1.6, CH), 7.46 (1H, t, J 7.6, CH), 7.56 (1H, dd, J 8.6, 2.2, CH), 7.64 (1H, d, J 8.8, CH), 7.79 (1H, d, J 2.4, CH), 7.93 (4H, s, br, 2×NH₂), 7.99 (1H, dd, J 7.2, 1.6, CH), 8.65 (1H, s, CH═N), 12.25 (1H, s, N—NH). HRMS (ESI) calcd. for C₁₅H₁₄Cl₃N₄O⁺ (M)⁺ 371.0228. found 371.0237.

### (E)-Amino(2-(2-chloro-3-((3,4-dichlorobenzyl)oxy)benzylidene)hydrazinyl) methaniminium chloride (51h)

69 mg, 84%, white solid, m.p. 230-233° C., ¹H NMR (400 MHz, DMSO-d₆): δ5.32 (2H, s, CH₂), 7.35 (1H, dd, J 8.4, 1.2, CH), 7.43 (1H, t, J 7.8, CH), 7.53 (1H, dd, J 8.0, 2.0, CH), 7.75 (1H, d, J 8.4, CH), 7.81 (1H, d, J 2.0, CH), 7.92 (4H, s, br, 2×NH₂), 7.96 (1H, dd, J 8.0, 1.2, CH), 8.65 (1H, s, CH═N), 12.25 (1H, s, N—NH). HRMS (ESI) calcd. for C₁₅H₁₄Cl₃N₄O⁺ (M)⁺ 371.0228. found 371.0239.

### (E)-Amino(2-(2-chloro-3-((2,3,5-trichlorobenzyl)oxy)benzylidene)hydrazinyl) methaniminium chloride (51i)

52 mg, 59%, white solid, m.p. 273-276° C., ¹H NMR (400 MHz, DMSO-d₆): δ5.36 (2H, s, CH₂), 7.42 (1H, dd, J 7.4, 2.0, CH), 7.46 (1H, t, J 7.6, CH), 7.77 (1H, d, J 2.4, CH), 7.96 (4H, s, br, 2×NH₂), 7.96 (1H, d, J 2.8, CH), 7.99 (1H, dd, J 7.4, 1.8, CH), 8.65 (1H, s, CH═N), 12.32 (1H, s, N—NH). HRMS (ESI) calcd. for C₁₅H₁₃Cl₄N₄O⁺ (M)⁺ 404.9838. found 404.9851.

### (E)-Amino(2-(2-chloro-3-((3-(trifluoromethyl)benzyl)oxy)benzylidene)hydrazinyl) methaniminium acetate (51j)

55 mg, 63%, white solid, m.p. 183-187° C., ¹H NMR (400 MHz, DMSO-d₆): δ1.91 (3H, s, CH₃), 5.37 (2H, s, CH₂), 6.62 (4H, s, br, 2×NH₂), 7.21 (1H, d, J 7.2, CH), 7.32 (1H, t, J 8.0, CH), 7.72 (1H, t, J 7.6, CH), 7.77 (1H, d, J 7.6, CH), 7.85 (2H, d, J 7.6, 2×CH), 7.91 (1H, s, CH), 8.42 (1H, s, CH═N). HRMS (ESI) calcd. for C₁₆H₁₅ClF₃N₄O⁺ (M)⁺ 371.0881. found 371.0889.

### (E)-Amino(2-(2-chloro-3-((4-(trifluoromethyl)benzyl)oxy)benzylidene)hydrazinyl) methaniminium chloride (51k)

65 mg, 80%, white solid, m.p. 245-247° C., ¹H NMR (400 MHz, DMSO-d₆): δ5.43 (2H, s, CH₂), 7.36 (1H, d, J 8.0, CH), 7.43 (1H, t, J 8.0, CH), 7.76 (2H, d, J 8.0, 2×CH), 7.85 (2H, d, J 7.6, 2×CH), 7.95 (4H, s, br, 2×NH₂), 7.96 (1H, d, J 7.6, CH), 7.99 (1H, dd, J 7.4, 1.8, CH), 8.66 (1H, s, CH═N), 12.31 (1H, s, N—NH). HRMS (ESI) calcd. for C₁₆H₁₅ClF₃N₄O⁺ (M)⁺ 371.0881. found 371.0887.

### (E)-Amino(2-(2-chloro-3-((3-(trifluoromethyl)benzyl)oxy)benzylidene)hydrazinyl) methaniminium chloride (51l)

69 mg, 84%, white powder, m.p. 198-201° C., ¹H NMR (400 MHz, DMSO-d₆): δ5.41 (2H, s, CH₂), 7.39 (1H, d, J 8.4, CH), 7.44 (1H, t, J 7.8, CH), 7.73 (1H, t, J 7.6, CH), 7.78 (1H, d, J 8.0, CH), 7.85 (1H, d, J 7.2, CH), 7.91 (1H, s, CH), 7.94 (4H, s, br, 2×NH₂), 7.97 (1H, d, J 7.6, CH), 8.65 (1H, s, CH═N), 12.33 (1H, s, N—NH). HRMS (ESI) calcd. for C₁₆H₁₅ClF₃N₄O⁺ (M)⁺ 371.0881. found 371.0891.

### (E)-Amino(2-(2-chloro-3-((3-chlorobenzyl)oxy)benzylidene)hydrazinyl) methaniminium chloride (51m)

75 mg, >99%, pale yellow glass, ¹H NMR (400 MHz, DMSO-d₆): δ5.31 (2H, s, CH₂), 7.35 (1H, dd, J 8.0, 1.2, CH), 7.41 (1H, t, J 8.0, CH), 7.43-7.48 (1H, m, CH), 7.48-7.51 (2H, m, 2×CH), 7.59 (1H, s, CH), 7.92 (4H, s, br, 2×NH₂), 7.94 (1H, dd, J 8.0, 1.2, CH), 8.65 (1H, s, CH═N), 12.23 (1H, s, N—NH). HRMS (ESI) calcd. for C₁₅H₁₅Cl₂N₄O⁺ (M)⁺ 337.0617. found 337.0629.

### General Procedure: Synthesis of Boc-Protected Aminobenzyl Guanidine Derivatives (52a-52b)

3-Aminobenzylamine or 4-aminobenzylamine (10 mmol) was dissolved in N,N-dimethylformamide (8 mL). DiBoc-S-methylthiurea (10.5 mmol) and triethylamine (20 mmol) were added successively at 0° C. The reaction mixture was stirred for 18 h at room temperature and then evaporated at 70° C. Water (180 mL) and brine (20 mL) were added and the mixture was extracted with diethyl ether (2×200 mL). The organic layer was dried (Na₂SO₄), filtered and concentrated in vacuum. Purification by flash column chromatography (dichloromethane, 100%) gave the corresponding Boc-protected aminobenzyl guanidine derivative 52a or 52b.

### tert-butyl N-[(1E)-{[(3-aminophenyl)methyl]amino}({[(tert-butoxy)carbonyl]imino})methyl]carbamate (52a)

2.85 g, 78%, white foam, ¹H NMR (400 MHz, DMSO-d₆): δ1.44 (9H, s, C(CH₃)₃), 1.53 (9H, s, C(CH₃)₃), 4.41 (2H, d, J 5.6 CH₂), 5.14 (2H, s, br, NH₂), 6.46 (1H, d, J 7.2, CH), 6.50 (1H, s, CH), 6.51 (1H, d, J 7.2, CH), 7.02 (1H, t, J 8.0, CH), 8.56 (1H, t, J 5.6, NH), 11.60 (1H, s, br, NH). HRMS (ESI) calcd. for C₁₈H₂₉N₄O₄⁺ (M+H)⁺ 365.2183. found 365.2189.

### tert-butyl N-[(1E)-{[(4-aminophenyl)methyl]amino}({[(tert-butoxy)carbonyl]imino})methyl]carbamate (52b)

2.98 g, 81%, white foam, ¹H NMR (400 MHz, DMSO-d₆): δ1.45 (9H, s, C(CH₃)₃), 1.50 (9H, s, C(CH₃)₃), 4.34 (2H, d, J 5.6 CH₂), 5.10 (2H, s, br, NH₂), 6.58 (2H, d, J 7.6, 2×CH), 7.03 (2H, d, J 8.0, 2×CH), 8.41 (1H, t, J 5.6, NH), 11.55 (1H, s, br, NH). HRMS (ESI) calcd. for C₁₈H₂₉N₄O₄⁺ (M)⁺ 365.2183. found 365.2187.

### General procedure: Synthesis of amino((aminobenzyl)amino)methaniminium 2,2,2-trifluoroacetate derivatives (53a-b)

Compound 52a or 52b (0.1 mmol) was dissolved in dichloromethane (0.8 mL) and trifluoroacetic acid (0.2 mL) was added at 0° C. The reaction mixture was stirred for 2 h at 0° C. and then concentrated to dryness to give the corresponding aminobenzyl guanidine derivative 53a or 53b.

### Amino((3-aminobenzyl)amino)methaniminium 2,2,2-trifluoroacetate (53a)

28 mg, >99%, orange-yellow glass, ¹H NMR (400 MHz, DMSO-d₆): δ4.37 (2H, d, J 6.0 CH₂), 6.88-6.96 (3H, m, 3×CH), 7.30 (1H, t, J 7.8, CH), 7.42 (4H, s, br, 2×NH₂), 8.16 (1H, t, J 6.0, NH). HRMS (ESI) calcd. for C₈H₁₃N₄⁺ (M)⁺ 165.1135. found 165.1141.

### Amino((4-aminobenzyl)amino)methaniminium 2,2,2-trifluoroacetate (53b)

28 mg, >99%, orange-yellow glass, ¹H NMR (400 MHz, DMSO-d₆): δ4.34 (2H, d, J 6.0 CH₂), 7.07 (2H, d, J 8.4, 2×CH), 7.27 (2H, d, J 8.4, 2×CH), 7.34 (4H, s, br, 2×NH₂), 8.09 (1H, t, J 6.0, NH). HRMS (ESI) calcd. for C₈H₁₃N₄⁺ (M)⁺ 165.1135. found 165.1139.

### General Procedure: Synthesis of Boc-Protected Sulphonamide Derivatives (54a-h)

Compound 52a or 52b (0.2 mmol) was dissolved in dichloromethane (1.2 mL) and pyridine (0.8 mL) at 0° C. The corresponding benzene sulphonyl chloride (0.22 mmol) was added and the reaction mixture was stirred for 2 h at 0° C. Water (40 mL) and brine (10 mL) were added and the mixture was extracted with diethyl ether (2×50 mL). The organic layer was dried (Na₂SO₄), filtered and concentrated to dryness. The residue was then co-evaporated with toluene (2×5 mL) and dichloromethane (2×5 mL) to give the corresponding Boc-protected sulphonamide 54a-h.

### tert-butyl N-[(1E)-{[(tert-butoxy)carbonyl]imino}({[4-(3-chloro benzenesulfonamido)phenyl]methyl}amino)methyl]carbamate (54a)

105 mg, 97%, pale yellow foam, ¹H NMR (400 MHz, CDCl₃): δ1.46 (9H, s, C(CH₃)₃), 1.47 (9H, s, C(CH₃)₃), 4.54 (2H, s, CH₂), 7.07 (2H, d, J 8.0, 2×CH), 7.13 (2H, d, J 8.4, 2×CH), 7.34 (1H, t, J 8.0, CH), 7.47 (1H, dd, J 8.0, 1.2, CH), 7.65 (1H, d, J 7.6, CH), 7.73 (1H, s, br, NH), 7.78 (1H, s, CH), 8.61 (1H, s, br, NH), 11.50 (1H, s, br, NH). HRMS (ESI) calcd. for C₂₄H₃₂ClN₄O₆S⁺ (M+H)⁺ 539.1726. found 539.1739.

### tert-butyl N-({[(tert-butoxy)carbonyl]imino}({[4-(4-chlorobenzenesulfonamido) phenyl]methyl}amino)methyl)carbamate (54b)

103 mg, 95%, white foam, ¹H NMR (400 MHz, CDCl₃): δ1.46 (9H, s, C(CH₃)₃), 1.47 (9H, s, C(CH₃)₃), 4.55 (2H, s, CH₂), 7.06 (2H, d, J 8.4, 2×CH), 7.14 (2H, d, J 8.4, 2×CH), 7.38 (2H, dt, J 8.8, 2.2, 2×CH), 7.60 (1H, s, br, NH), 7.71 (2H, dd, J 8.8, 2.2, 2×CH), 8.58 (1H, s, br, NH), 11.51 (1H, s, br, NH). HRMS (ESI) calcd. for C₂₄H₃₂ClN₄O₆S⁺ (M+H)⁺ 539.1726. found 539.1741.

### tert-butyl N-({[(tert-butoxy)carbonyl]imino}({[4-(2,3-dichlorobenzene sulfonamido)phenyl]methyl}amino)methyl)carbamate (54c)

113 mg, 98%, yellow foam, ¹H NMR (400 MHz, CDCl₃): δ1.45 (9H, s, C(CH₃)₃), 1.47 (9H, s, C(CH₃)₃), 4.51 (2H, s, CH₂), 7.08 (2H, d, J 8.8, 2×CH), 7.13 (2H, d, J 8.4, 2×CH), 7.27 (1H, t, J 8.2, CH), 7.49 (1H, s, br, NH), 7.61 (1H, dd, J 8.0, 1.6, CH), 7.93 (1H, dd, J 8.0, 1.6, CH), 8.51 (1H, s, br, NH), 11.50 (1H, s, br, NH). HRMS (ESI) calcd. for C₂₄H₃₁Cl₂N₄O₆S⁺ (M+H)⁺ 573.1336. found 573.1347.

### tert-butyl N-[(1E)-{[(tert-butoxy)carbonyl]imino}[({4-[3-(trifluoromethyl) benzenesulfonamido]phenyl}methyl)amino]methyl]carbamate (54d)

111 mg, 97%, pale yellow foam, ¹H NMR (400 MHz, CDCl₃): δ1.47 (9H, s, C(CH₃)₃), 1.48 (9H, s, C(CH₃)₃), 4.57 (2H, s, CH₂), 7.07 (2H, d, J 8.4, 2×CH), 7.13 (2H, d, J 8.4, 2×CH), 7.56 (1H, t, J 7.8, CH), 7.69 (1H, s, br, NH), 7.76 (1H, d, J 8.0, CH), 7.96 (1H, d, J 8.0, CH), 8.04 (1H, s, CH), 8.66 (1H, s, br, NH), 11.50 (1H, s, br, NH). HRMS (ESI) calcd. for C₂₅H₃₂F₃N₄O₆S⁺ (M+H)⁺ 573.1989. found 573.1996.

### tert-butyl N-[(1E)-{[(tert-butoxy)carbonyl]imino}({[3-(3-chlorobenzene sulfonamido)phenyl]methyl}amino)methyl]carbamate (54e)

99 mg, 92%, white foam, ¹H NMR (400 MHz, CDCl₃): δ1.47 (9H, s, C(CH₃)₃), 1.48 (9H, s, C(CH₃)₃), 4.51 (2H, s, CH₂), 6.98-7.06 (3H, m, 3×CH), 7.18 (1H, t, J 8.0, CH), 7.34 (1H, t, J 7.8, CH), 7.46 (1H, ddd, J 8.0, 2.0, 0.8, CH), 7.54 (1H, s, br, NH), 7.64 (1H, ddd, J 7.8, 1.6, 1.2, CH), 7.79 (1H, t, J 1.8, CH), 8.60 (1H, s, br, NH), 11.52 (1H, s, br, NH). HRMS (ESI) calcd. for C₂₄H₃₂ClN₄O₆S⁺ (M+H)⁺ 539.1726. found 539.1737.

### tert-butyl N-[(1E)-{[(tert-butoxy)carbonyl]imino}({[3-(4-chlorobenzene sulfonamido)phenyl]methyl}amino)methyl]carbamate (54f)

98 mg, 91%, foam, ¹H NMR (400 MHz, CDCl₃): δ1.47 (9H, s, C(CH₃)₃), 1.48 (9H, s, C(CH₃)₃), 4.53 (2H, s, CH₂), 7.00 (1H, d, J 2.4, CH), 7.02 (1H, d, J 2.4, CH), 7.07 (1H, s, CH), 7.17 (1H, t, J 7.8, CH), 7.35 (2H, dt, J 8.8, 2.2, 2×CH), 7.65 (1H, s, br, NH), 7.70 (2H, dt, J 8.8, 2.2, 2×CH), 8.61 (1H, s, br, NH), 11.52 (1H, s, br, NH). HRMS (ESI) calcd. for C₂₄H₃₂ClN₄O₆S⁺ (M+H)⁺ 539.1726. found 539.1738.

### tert-butyl N-[(1E)-{[(tert-butoxy)carbonyl]imino}({[3-(2,3-dichlorobenzene sulfonamido)phenyl]methyl}amino)methyl]carbamate (54g)

107 mg, 93%, yellow foam, ¹H NMR (400 MHz, CDCl₃): δ1.48 (9H, s, C(CH₃)₃), 1.49 (9H, s, C(CH₃)₃), 4.55 (2H, s, CH₂), 6.98-7.04 (2H, m, 2×CH), 7.08 (1H, s, CH), 7.17 (1H, t, J 7.8, CH), 7.27 (1H, t, J 8.0, CH), 7.46 (1H, s, br, NH), 7.60 (1H, d, J 8.0, CH), 7.97 (1H, d, J 8.0, CH), 8.61 (1H, s, br, NH), 11.52 (1H, s, br, NH). HRMS (ESI) calcd. for C₂₄H₃₁Cl₂N₄O₆S⁺ (M+H)⁺ 573.1336. found 573.1349.

### tert-butyl N-[(1E)-{[(tert-butoxy)carbonyl]imino}[({3-[3-(trifluoromethyl) benzenesulfonamido]phenyl}methyl)amino]methyl]carbamate (54h)

107 mg, 93%, colourless glass, ¹H NMR (400 MHz, CDCl₃): δ1.47 (18H, s, 2×C(CH₃)₃), 4.52 (2H, s, CH₂), 6.99-7.07 (3H, m, 3×CH), 7.18 (1H, t, J 7.8, CH), 7.55 (1H, t, J 7.8, CH), 7.68 (1H, s, br, NH), 7.75 (1H, d, J 7.6, CH), 7.95 (1H, d, J 7.6, CH), 8.05 (1H, s, CH), 8.62 (1H, s, br, NH), 11.51 (1H, s, br, NH). HRMS (ESI) calcd. for C₂₅H₃₂F₃N₄O₆S⁺ (M+H)⁺ 573.1989. found 573.1998.

### General procedure: Synthesis of Amino((4-phenylsulfonamido)benzyl) amino)methaniminium 2,2,2-trifluoroacetate derivatives (55a-h)

Compound 54a-h (0.1 mmol) was dissolved in dichloromethane (0.8 mL) and trifluoroacetic acid (0.2 mL) was added and the reaction mixture was stirred for 2 h at room temperature. The mixture was concentrated to dryness to give the corresponding guanidine trifluoroacetate salts 55a-h.

### Amino((4-((3-chlorophenyl)sulfonamido)benzyl)amino)methaniminium 2,2,2-trifluoroacetate (55a)

90 mg, 99%, glass, ¹H NMR (400 MHz, CD₃OD): δ4.38 (2H, s, CH₂), 7.17-7.22 (2H, m, 2×CH), 7.24-7.31 (2H, m, 2×CH), 7.53 (1H, t, J 7.4, CH), 7.60-7.66 (1H, m, CH), 7.71-7.77 (1H, m, CH), 7.77-7.81 (1H, m, CH). HRMS (ESI) calcd. for C₁₄H₁₆ClN₄O₂S⁺ (M)⁺ 339.0677. found 339.0685.

### Amino((4-((4-chlorophenyl)sulfonamido)benzyl)amino)methaniminium 2,2,2-trifluoroacetate (55b)

89 mg, 98%, glass, ¹H NMR (400 MHz, CD₃OD): δ4.38 (2H, s, CH₂), 7.20 (2H, d, J 8.4, 2×CH), 7.27 (2H, d, J 8.4, 2×CH), 7.55 (2H, d, J 8.4, 2×CH), 7.80 (2H, d, J 8.4, 2×CH). HRMS (ESI) calcd. for C₁₄H₁₆ClN₄O₂S⁺ (M)⁺ 339.0677. found 339.0686.

### Amino((4-((2,3-dichlorophenyl)sulfonamido)benzyl)amino)methaniminium 2,2,2-trifluoroacetate (55c)

97 mg, >99%, glass, ¹H NMR (400 MHz, CD₃OD): δ4.34 (2H, s, CH₂), 7.21-7.28 (4H, m, 2×CH), 7.46 (1H, t, J 8.0, CH), 7.81 (1H, dd, J 8.0, 1.6, CH), 8.10 (1H, dd, J 8.0, 1.6, CH). HRMS (ESI) calcd. for C₁₄H₁₅Cl₂N₄O₂S⁺ (M)⁺ 373.0287. found 373.0296.

### Amino((4-((3-trifluoromethyl)phenyl)sulfonamido)benzyl)amino) methaniminium 2,2,2-trifluoroacetate (55d)

95 mg, 97%, glass, ¹H NMR (400 MHz, CD₃OD): δ4.38 (2H, s, CH₂), 7.20 (2H, d, J 8.8, 2×CH), 7.29 (2H, d, J 8.4, 2×CH), 7.76 (1H, t, J 8.2, CH), 7.95 (1H, d, J 8.0, CH), 8.05-8.11 (2H, m, 2×CH). HRMS (ESI) calcd. for C₁₅H₁₆F₃N₄O₂S⁺ (M)⁺ 373.0941. found 373.0945.

### Amino((3-((3-chlorophenyl)sulfonamido)benzyl)amino)methaniminium 2,2,2-trifluoroacetate (55e)

89 mg, 98%, glass, ¹H NMR (400 MHz, CD₃OD): δ4.37 (2H, s, CH₂), 7.07 (1H, d, J 8.0, CH), 7.13 (1H, d, J 7.6, CH), 7.23 (1H, s, CH), 7.33 (1H, t, J 7.8, CH), 7.54 (1H, t, J 8.0, CH), 7.65 (1H, d, J 8.0, CH), 7.75 (1H, d, J 8.0, CH), 7.81 (1H, s CH). HRMS (ESI) calcd. for C₁₄H₁₆ClN₄O₂S⁺ (M)⁺ 339.0677. found 339.0681.

### Amino((3-((4-chlorophenyl)sulfonamido)benzyl)amino)methaniminium 2,2,2-trifluoroacetate (55f)

88 mg, 97%, glass, ¹H NMR (400 MHz, CD₃OD): δ4.38 (2H, s, CH₂), 7.07 (1H, d, J 8.0, CH), 7.11 (1H, d, J 7.6, CH), 7.23 (1H, s, CH), 7.31 (1H, t, J 8.0, CH), 7.54 (2H, d, J 8.4, 2×CH), 7.81 (2H, d, J 8.4, CH). HRMS (ESI) calcd. for C₁₄H₁₆ClN₄O₂S⁺ (M)⁺ 339.0677. found 339.0685.

### Amino((3-((2,3-dichlorophenyl)sulfonamido)benzyl)amino)methaniminium 2,2,2-trifluoroacetate (55g)

96 mg, 98%, glass, ¹H NMR (400 MHz, CD₃OD): δ4.39 (2H, s, CH₂), 7.06 (1H, d, J 7.6, CH), 7.13 (1H, d, J 8.0, CH), 7.22 (1H, s, CH), 7.30 (1H, t, J 8.0, CH), 7.49 (1H, dt, J 8.2, 0.8, CH), 7.81 (1H, d, J 8.0, CH), 8.11 (1H, d, J 8.0, CH). HRMS (ESI) calcd. for C₁₄H₁₅Cl₂N₄O₂S⁺ (M)⁺ 373.0287. found 373.0295.

### Amino((3-((3-(trifluoromethyl)phenyl)sulfonamido)benzyl)amino) methaniminium 2,2,2-trifluoroacetate (55h)

95 mg, 97%, glass, ¹H NMR (400 MHz, CD₃OD): δ4.42 (2H, s, CH₂), 7.07 (1H, d, J 8.0, CH), 7.14 (1H, d, J 7.6, CH), 7.24 (1H, s, CH), 7.32 (1H, t, J 8.0, CH), 7.77 (1H, d, J 8.2, CH), 7.96 (1H, d, J 7.6, CH), 8.07 (2H, s, 2×CH). HRMS (ESI) calcd. for C₁₅H₁₆F₃N₄O₂S⁺ (M)⁺ 373.0941. found 373.0946.

### General Procedure: Synthesis of Boc-Protected Amide Derivatives (56a-b)

Compound 52a or 52 b (0.5 mmol) and potassium carbonate (1.0 mmol) were placed in an oven-dried 50 mL-glass tube. Acetone (2.0 mL) and benzoyl chloride (0.75 mmol) were added successively and the reaction mixture was stirred for 18 h at 80° C. Water (80 mL) and brine (20 mL) were added and the mixture was extracted with diethyl ether (2×100 mL). The organic layer was dried (Na₂SO₄), filtered and concentrated to dryness. Purification by flash column chromatography (pentane to pentane/ethyl acetate 4:1) gave 56a-b.

### tert-butyl N-[(1E)-{[(3-benzamidophenyl)methyl]amino}({[(tert-butoxy) carbonyl]imino})methyl]carbamate (56a)

45 mg, 19%, colourless glass, ¹H NMR (400 MHz, CDCl₃): δ1.47 (9H, s, C(CH₃)₃), 1.48 (9H, s, C(CH₃)₃), 4.59 (2H, d, J 5.2 CH₂), 7.01 (1H, d, J 7.6, CH), 7.30 (1H, t, J 8.0, CH), 7.42-7.49 (3H, m, 3×CH), 7.53 (1H, t, J 7.6, CH), 7.70 (1H, t, J 8.0, CH), 7.87-7.93 (2H, m, 2×CH), 8.19 (1H, s, NH), 8.70 (1H, s, NH), 11.53 (1H, s, br, NH). HRMS (ESI) calcd. for C₂₅H₃₃N₄O₅⁺ (M+H)⁺ 469.2445. found: 469.2452.

### tert-butyl N-[(1E)-{[(4-benzamidophenyl)methyl]amino}({[(tert-butoxy) carbonyl]imino})methyl]carbamate (56b)

27 mg, 11%, colourless glass, ¹H NMR (400 MHz, CDCl₃): δ1.47 (9H, s, C(CH₃)₃), 1.48 (9H, s, C(CH₃)₃), 4.58 (2H, d, J 5.2 CH₂), 7.24 (2H, d, J 8.4, 2×CH), 7.45 (2H, t, J 7.4, CH), 7.52 (1H, t, J 7.2, CH), 7.61 (2H, d, J 8.8, CH), 7.87 (2H, d, J 8.4, 2×CH), 8.14 (1H, s, NH), 8.63 (1H, s, NH), 11.53 (1H, s, br, NH). HRMS (ESI) calcd. for C₂₅H₃₃N₄O₅⁺ (M+H)⁺ 469.2445. found: 469.2451.

### General procedure: Synthesis of amino((benzamidobenzyl)amino) methaniminium 2,2,2-trifluoroacetate derivatives (57a-b)

Compound 56a or 56b (0.1 mmol) were dissolved in dichloromethane (0.8 mL) and trifluoroacetic acid (0.2 mL) was added and the reaction mixture was stirred for 2 h at 0° C. The mixture was concentrated to dryness to give the corresponding guanidine trifluoroacetate salt 57a or 57b.

### Amino((3-benzamidobenzyl)amino)methaniminium 2,2,2-trifluoroacetate (57a)

38 mg, 99%, pale yellow glass, ¹H NMR (400 MHz, DMSO-d₆): δ4.44 (2H, s, CH₂), 7.10 (1H, d, J 8.0, CH), 7.42 (1H, t, J 8.0, CH), 7.56-7.62 (3H, m, 3×CH), 7.65 (1H, d, J 7.2, CH), 7.90 (1H, s, CH), 8.00 (2H, d, J 7.2, 2×CH), 8.01 (1H, s, NH). HRMS (ESI) calcd. for C₁₅H₁₇N₄O⁺ (M)⁺ 269.1397. found: 269.1401.

### Amino((4-benzamidobenzyl)amino)methaniminium 2,2,2-trifluoroacetate (57b)

38 mg, 99%, pale yellow glass, ¹H NMR (400 MHz, DMSO-d₆): δ4.39 (2H, s, CH₂), 7.35 (2H, d, J 8.4, 2×CH), 7.59 (2H, t, J 7.2, 2×CH), 7.66 (1H, t, J 7.2, CH), 7.84 (2H, d, J 8.4, 2×CH), 8.00 (2H, d, J 8.0, 2×CH), 8.01 (1H, s, NH). HRMS (ESI) calcd. for C₁₅H₁₇N₄O⁺ (M)⁺ 269.1397. found: 269.1402.

### General Procedure: Synthesis of Boc-Protected Amide/Sulphonamide Derivative (58a-b)

4-Aminobenzylamine (0.5 mmol) was dissolved in N,N-dimethylformamide (1.0 mL) and triethylamine (2.0 mmol). Benzoyl chloride or benzenesulphonyl chloride (0.5 mmol) was added successively and the reaction mixture was stirred for 2 h at 0° C. Mercury chloride (0.5 mmol) and Di-Boc-S-methylthiurea (0.5 mmol) were added and the reaction mixture was stirred for 18 h at room temperature. Diethyl ether (100 mL) was added and the mixture was filtered through a paper filter. The organic layer was washed with water (80 mL) and brine (20 mL), dried (NaCl), filtered and concentrated to dryness. Crystallisation from diethyl ether the corresponding Boc-protected amide/sulphonamide derivatives 58a-b.

### tert-butyl N-[(1Z)-{[(tert-butoxy)carbonyl]imino}({4-[(phenylformamido)methyl]phenyl}amino)methyl]carbamate (58a)

113 mg, 48%, white solid, m.p. 152-156° C., mixture of conformers by ¹H NMR. Major conformer: ¹H NMR (400 MHz, CDCl₃): 1.45 (9H, s, C(CH₃)₃), 1.53 (9H, s, C(CH₃)₃), 4.55 (2H, d, J 5.2, CH₂), 6.64 (1H, s, br, NH), 7.25 (2H, d, J 8.0, 2×CH), 7.39-7.52 (5H, m, 5×CH), 7.81 (2H, d, J 8.0, 2×CH), 10.40 (1H, s, br, NH), 11.66 (1H, s, br, NH). HRMS (ESI) calcd. for C₂₅H₃₃N₄O₅⁺ (M+H)⁺ 469.2445. found: 469.2451.

### tert-butyl N-[(1Z)-{[4-(benzenesulfonamidomethyl)phenyl]amino}({[(tert-butoxy)carbonyl]imino})methyl]carbamate (58b)

128 mg, 50%, white solid, m.p. 154-158° C., ¹H NMR (400 MHz, CDCl₃): δ1.47 (9H, s, C(CH₃)₃), 1.53 (9H, s, C(CH₃)₃), 4.10 (2H, d, J 6.0, CH₂), 4.90 (1H, s, br, NH), 7.17 (2H, d, J 8.0, 2×CH), 7.46 (2H, d, J 8.4, 2×CH), 7.49-7.55 (2H, m, 2×CH), 7.56-7.62 (1H, m, CH), 7.87 (1H, d, J 1.6, CH), 7.89 (1H, s, CH), 10.51 (1H, s, br, NH), 11.65 (1H, s, br, NH). HRMS (ESI) calcd. for C₂₄H₃₃N₄O₆S⁺ (M+H)⁺ 505.2115. found: 505.2127.

### General procedure: Synthesis of Amino((4-(benzamidomethyl)phenyl)amino) methaniminium 2,2,2-trifluoroacetate (59a) and Amino((4-(phenyl sulfonamidomethyl)phenyl)amino)methaniminium 2,2,2-trifluoroacetate (59b)

Compound 58a or 58b (0.1 mmol) was dissolved in dichloromethane (0.8 mL) and trifluoroacetic acid (0.2 mL) was added and the reaction mixture was stirred for 2 h at 0° C. The mixture was concentrated to dryness to give the corresponding amide/sulphonamide guanidine trifluoroacetate salt 59a-b.

### Amino((4-(benzamidomethyl)phenyl)amino)methaniminium 2,2,2-trifluoroacetate (59a)

38 mg, 99%, pale yellow glass, mixture of conformers by ¹H NMR. Major conformer: ¹H NMR (400 MHz, DMSO-d₆): δ4.54 (2H, s, CH₂), 7.25 (2H, d, J 8.0, 2×CH), 7.44 (4H, s, br, 2×NH₂), 7.45 (2H, d, J 8.0, 2×CH), 7.56-7.62 (1H, m, CH), 7.53 (2H, t, J 7.2, 2×CH), 7.94 (2H, d, J 7.2, 2×CH), 7.95 (1H, s, NH), 9.17 (1H, t, J 5.4, NH), 9.79 (1H, s, NH). HRMS (ESI) calcd. for C₁₅H₁₇N₄O⁺ (M)⁺ 269.1397. found: 269.1405.

### Amino((4-(phenylsulfonamidomethyl)phenyl)amino)methaniminium 2,2,2-trifluoroacetate (59b)

38 mg, 99%, pale yellow glass, ¹H NMR (400 MHz, DMSO-d₆): δ4.03 (2H, d, J 6.4, CH₂), 7.21 (2H, d, J 8.0, 2×CH), 7.37 (2H, d, J 8.0, 2×CH), 7.45 (4H, s, br, 2×NH₂), 7.61-7.72 (3H, m, CH), 7.87 (2H, d, J 7.2, 2×CH), 8.27 (1H, t, J 6.4, NH), 9.78 (1H, s, br, NH). HRMS (ESI) calcd. for C₁₄H₁₇N₄O₂S⁺ (M)⁺ 305.1067. found: 305.1078.

### General Procedure: Synthesis of 1-benzyl-1H-imidazole-2-carboxaldehyde Derivatives (60a-d) and 1-benzyl-1H-pyrrole-2-carboxaldehyde Derivatives (60e-h)

2-Imidazolecarboxaldehyde (1 mmol) or pyrrole-2-carboxaldehyde (1 mmol) and potassium carbonate (4 mmol) were placed in a 25 mL round-bottom flask. N,N-dimethylformamide (1 mL) was added. Then the corresponding benzyl halide (1.2 mmol) was added and the reaction mixture was stirred for 18 h at room temperature. Water (80 mL) and brine (20 mL) were added and the mixture was extracted with diethyl ether (100 mL). The organic layer was dried (NaCl), filtered and concentrated in vacuo. All derivatives were purified by column chromatography.

### 1-(3-Chlorobenzyl)-1H-imidazole-2-carbaldehyde (60a)

Purification by flash column chromatography (dichloromethane to dichloromethane/ethyl acetate 9:1) gave 60a (177 mg, 80%, colourless oil). ¹H NMR (400 MHz, CDCl₃): δ5.58 (2H, s, CH₂), 7.05-7.08 (1H, m, CH), 7.15 (2H, s, 2×CH), 7.25-7.28 (2H, m, 2×CH), 7.32 (1H, s, CH), 9.84 (1H, d, J 0.5, CH═O). HRMS (ESI) calcd. for C₁₁H₁₀ClN₂O⁺ (M+H)⁺ 221.0476. found 221.0469.

### 1-(4-Chlorobenzyl)-1H-imidazole-2-carbaldehyde (60b)

Purification by flash column chromatography (dichloromethane to dichloromethane/ethyl acetate 9:1) gave 60b (184 mg, 83%, beige solid). ¹H NMR (400 MHz, CDCl₃): δ5.57 (2H, s, CH₂), 7.11-7.13 (1H, m, CH), 7.13-7.15 (2H, m, 2×CH), 7.30 (2H, dt, J 8.4, 2.2, 2×CH), 7.32 (1H, s, CH), 9.86 (1H, s, CH═O). HRMS (ESI) calcd. for C₁₁H₁₀ClN₂O⁺ (M+H)⁺ 221.0476. found 221.0471.

### 1-(3-(Trifluoromethyl)benzyl)-1H-imidazole-2-carbaldehyde (60c)

Purification by flash column chromatography (dichloromethane to dichloromethane/ethyl acetate 9:1) gave 60c (197 mg, 77%, colourless oil). ¹H NMR (400 MHz, DMSO-d₆): δ5.75 (2H, s, CH₂), 7.42 (1H, d, J 0.8, CH), 7.52 (1H, d, J 8.0, CH), 7.62-7.67 (2H, m, 2×CH), 7.70-7.74 (1H, m, CH), 7.90 (1H, s, CH), 9.75 (1H, d, J 0.8, CH═O). HRMS (ESI) calcd. for C₁₂H₁₀F₃N₂O⁺ (M+H)⁺ 255.0740. found 255.0747.

### 1-(4-(Trifluoromethyl)benzyl)-1H-imidazole-2-carbaldehyde (60d)

Purification by flash column chromatography (dichloromethane to dichloromethane/ethyl acetate 9:1) gave 60d (100 mg, 39%, beige solid). ¹H NMR (400 MHz, CDCl₃): δ5.68 (2H, s, CH₂), 7.19 (1H, s, CH), 7.29 (2H, d, J 8.0, 2×CH), 7.37 (1H, s, CH), 7.60 (2H, d, J 8.0, 2×CH), 9.88 (1H, s, CH═O). HRMS (ESI) calcd. for C₁₂H₁₀F₃N₂O⁺ (M+H)⁺ 255.0740. found 255.0749.

### 1-(3-Chlorobenzyl)-1H-pyrrole-2-carbaldehyde (60e)

Purification by flash column chromatography (pentane to pentane/ethyl acetate 9:1) gave 60e (165 mg, 75%, colourless oil). ¹H NMR (400 MHz, CDCl₃): δ5.53 (2H, s, CH₂), 6.29 (1H, dd, J 3.8, 2.6, CH), 6.96-6.99 (2H, m, 2×CH), 7.07-7.09 (1H, m, CH), 7.21-7.23 (2H, m, 2×CH), 7.25-7.27 (1H, m, CH), 9.54 (1H, d, J 0.8, CH═O). HRMS (ESI) calcd. for C₁₂H₁₁ClNO⁺ (M+H)⁺ 220.0524. found 220.0519.

### 1-(4-Chlorobenzyl)-1H-pyrrole-2-carbaldehyde (60f)

Purification by flash column chromatography (pentane to pentane/ethyl acetate 9:1) gave 60f (185 mg, 84%, beige solid). ¹H NMR (400 MHz, CDCl₃): δ5.51 (2H, s, CH₂), 6.28 (1H, t, J 3.2, CH), 6.95-6.98 (2H, m, 2×CH), 7.07 (2H, dt, J 8.4, 2.2, 2×CH), 7.26 (2H, dt, J 8.4, 2.2, 2×CH), 9.54 (1H, s, CH═O). HRMS (ESI) calcd. for C₁₂H₁₁ClNO⁺ (M+H)⁺ 220.0524. found 220.0521.

### 1-(3-(Trifluoromethyl)benzyl)-1H-pyrrole-2-carbaldehyde (60g)

Purification by flash column chromatography (pentane to pentane/ethyl acetate 9:1) gave 60g (216 mg, 85%, beige-brown oil). ¹H NMR (400 MHz, CDCl₃): δ5.61 (2H, s, CH₂), 6.31 (1H, t, J 3.2, CH), 6.98-7.01 (2H, m, 2×CH), 7.30 (1H, d, J 7.6, CH), 7.37 (1H, s, CH), 7.42 (1H, t, J 7.8, CH), 7.52 (1H, d, J 8.0, CH), 9.55 (1H, s, CH═O). HRMS (ESI) calcd. for C₁₃H₁₁F₃NO⁺ (M+H)⁺ 254.0787. found 254.0793.

### 1-(4-(Trifluoromethyl)benzyl)-1H-pyrrole-2-carbaldehyde (60h)

Purification by flash column chromatography (pentane to pentane/ethyl acetate 9:1) gave 60h (82 mg, 32%, beige solid). ¹H NMR (400 MHz, CDCl₃): δ5.61 (2H, s, CH₂), 6.31 (1H, t, J 3.0, CH), 6.98-7.01 (2H, m, 2×CH), 7.21 (2H, d, J 8.0, 2×CH), 7.55 (2H, d, J 8.0, 2×CH), 9.54 (1H, s, CH═O). HRMS (ESI) calcd. for C₁₃H₁₁F₃NO⁺ (M+H)⁺ 254.0787. found 254.0795.

### General procedure: Synthesis of (E)-amino(2-((1-benzyl-1H-imidazol-2-yl)methylene)hydrazinyl)methaniminium chloride derivatives (61a-d) and (E)-Amino(2-((1-benzyl-1H-pyrrol-2-yl)methylene)hydrazinyl)methaniminium chloride derivatives (61e-h)

Aldehyde derivatives (60a-h) (0.2 mmol) and N-aminoguanidine hydrocarbonate (0.24 mmol) were placed in a 50 mL round-bottom flask. HCL (0.5M in methanol, 2.0 mL) was added and the reaction mixture was stirred at 80° C. for 2 h and then evaporated to dryness.

### (E)-amino(2-((1-(3-chlorobenzyl)-1H-imidazol-2-yl)methylene)hydrazinyl) methaniminium chloride (61a)

87 mg, >99%, pale yellow glass, ¹H NMR (400 MHz, DMSO-d₆): δ5.71 (2H, s, CH₂), 7.35 (1H, dt, J 4.4, 1.6, CH), 7.46-7.50 (2H, m, 2×CH), 7.52 (1H, s, CH), 7.88 (1H, d J 2.2, CH), 7.94 (1H, d J 2.2, CH), 8.35 (4H, s, br, 2×NH₂), 8.56 (1H, s, CH═N), 12.85 (1H, s, br, N—NH). HRMS (ESI) calcd. for C₁₂H₁₄ClN₆⁺ (M)⁺ 277.0963. found 277.0956.

### (E)-Amino(2-((1-(4-chlorobenzyl)-1H-imidazol-2-yl)methylene)hydrazinyl) methaniminium chloride (61b)

88 mg, >99%, pale yellow glass, ¹H NMR (400 MHz, DMSO-d₆): δ5.70 (2H, s, CH₂), 7.42 (2H, dt, J 8.4, 2.2, 2×CH), 7.52 (2H, dt, J 8.4, 2.2, 2×CH), 7.86 (1H, d J 1.8, CH), 7.91 (1H, d J 1.8, CH), 8.35 (4H, s, br, 2×NH₂), 8.53 (1H, s, CH═N), 12.82 (1H, s, br, N—NH). HRMS (ESI) calcd. for C₁₂H₁₄ClN₆⁺ (M)⁺ 277.0963. found 277.0955.

### (E)-Amino(2-((1-(3-(trifluoromethyl)benzyl)-1H-imidazol-2-yl)methylene) hydrazinyl)methaniminium chloride (61c)

94 mg, >99%, pale yellow glass, ¹H NMR (400 MHz, DMSO-d₆): δ5.80 (2H, s, CH₂), 7.65 (1H, d, J 7.8, CH), 7.70 (1H, t, J 7.8, CH), 7.80 (1H, d, J 7.6, CH), 7.85 (1H, s, CH), 7.90 (1H, d J 1.8, CH), 7.94 (1H, d J 1.8, CH), 8.35 (4H, s, br, 2×NH₂), 8.59 (1H, s, CH═N), 12.85 (1H, s, br, N—NH). HRMS (ESI) calcd. for C₁₃H₁₄F₃N₆⁺ (M)⁺ 311.1227. found 311.1234.

### (E)-Amino(2-((1-(4-(trifluoromethyl)benzyl)-1H-imidazol-2-yl)methylene) hydrazinyl)methaniminium chloride (61d)

95 mg, >99%, pale yellow glass, ¹H NMR (400 MHz, DMSO-d₆): δ5.83 (2H, s, CH₂), 7.58 (2H, d, J 8.0, 2×CH), 7.82 (2H, d, J 8.0, 2×CH), 7.88 (1H, d J 1.6, CH), 7.94 (1H, d J 1.6, CH), 8.29 (4H, s, br, 2×NH₂), 8.51 (1H, s, CH═N), 12.73 (1H, s, br, N—NH). HRMS (ESI) calcd. for C₁₃H₁₄F₃N₆⁺ (M)⁺ 311.1227. found 311.1235.

### (E)-Amino(2-((1-(3-chlorobenzyl)-1H-pyrrol-2-yl)methylene)hydrazinyl) methaniminium chloride (61e)

64 mg, >99%, purple-black glass, ¹H NMR (400 MHz, DMSO-d₆): δ5.61 (2H, s, CH₂), 6.29 (1H, dd, J 3.8, 2.6, CH), 6.77 (1H, dd, J 3.8, 1.8, CH), 7.01 (1H, dt, J 7.2, 1.6, CH), 7.07 (1H, t, J 1.6, CH), 7.26 (1H, dd, J 2.4, 2.0, CH), 7.35 (1H, dt, J 8.0, 1.8, CH), 7.39 (1H, t, J 7.6, CH), 7.59 (4H, s, br, 2×NH₂), 8.09 (1H, s, CH═N), 11.55 (1H, s, N—NH). HRMS (ESI) calcd. for C₁₃H₁₅ClN₅⁺ (M)⁺ 276.1010. found 276.1006.

### (E)-Amino(2-((1-(4-chlorobenzyl)-1H-pyrrol-2-yl)methylene)hydrazinyl) methaniminium chloride (61f)

64 mg, >99%, purple-black glass, ¹H NMR (400 MHz, DMSO-d₆): δ5.58 (2H, s, CH₂), 6.27 (1H, dd, J 3.8, 1.4, CH), 6.76 (1H, dd, J 3.8, 1.8, CH), 7.07 (2H, d, J 8.0, 2×CH), 7.25 (1H, dd, J 6.4, 1.6, CH), 7.42 (2H, d, J 8.0, 2×CH), 7.49 (4H, s, br, 2×NH₂), 8.07 (1H, s, CH═N), 11.54 (1H, s, N—NH). HRMS (ESI) calcd. for C₁₃H₁₅ClN₅⁺ (M)⁺ 276.1010. found 276.1004.

### (E)-Amino(2-((1-(3-(trifluoromethyl)benzyl)-1H-pyrrol-2-yl)methylene) hydrazinyl)methaniminium chloride (61g)

81 mg, >99%, purple-black glass, ¹H NMR (400 MHz, DMSO-d₆): δ5.72 (2H, s, CH₂), 6.30 (1H, dd, J 3.8, 2.6, CH), 6.77 (1H, dd, J 3.8, 1.8, CH), 7.28-7.32 (2H, m, 2×CH), 7.44 (1H, s, CH), 7.50 (4H, s, br, 2×NH₂), 7.60 (1H, t, J 7.8, CH), 7.65 (1H, d, J 7.6, CH), 8.08 (1H, s, CH═N), 11.57 (1H, s, N—NH). HRMS (ESI) calcd. for C₁₄H₁₅F₃N₅⁺ (M)⁺ 310.1274. found 310.1278.

### (E)-Amino(2-((1-(4-(trifluoromethyl)benzyl)-1H-pyrrol-2-yl)methylene) hydrazinyl)methaniminium chloride (61h)

80 mg, >99%, purple-black glass, ¹H NMR (400 MHz, DMSO-d₆): δ5.71 (2H, s, CH₂), 6.31 (1H, t, J 2.8, CH), 6.78-6.79 (1H, m, CH), 7.23 (2H, d, J 8.0, 2×CH), 7.27 (1H, t, J 2.6, CH), 7.46 (4H, s, br, 2×NH₂), 7.73 (2H, d, J 8.0, 2×CH), 7.95 (1H, dd, J 7.6, 1.6, CH), 8.07 (1H, s, CH═N), 11.57 (1H, s, N—NH). HRMS (ESI) calcd. for C₁₄H₁₅F₃N₅⁺ (M)⁺ 310.1274. found 310.1281.

Biological Results

Minimum Inhibitory Concentrations (MICs) for the compounds prepared were determined against Escherichia coli (strain JM109) and Staphylococcus aureus (strain 9518) using the protocols described by Andrews (J. Antimicrob. Chemother., 2001, 48, Suppl. S1, 5-16).

| | MIC | | | MIC | | | MIC | |
|---|---|---|---|---|---|---|---|---|
| Compound | E. coli | S. aureus | Compound | E. coli | S. aureus | Compound | E. coli | S. aureus |
| 49a | +++ | +++ | 55c | + | + | 23c | − | ++ |
| 49b | +++ | +++ | 55d | + | + | 4n | − | + |
| 38 | ++ | ++ | 55e | + | + | 19b | − | + |
| 33 | +++ | +++ | 55f | + | + | 28 | − | + |
| 10a | + | ++ | 55g | + | + | 23b | − | − |
| 9b | ++ | ++ | 55h | + | + | 41a | − | − |
| 15 | ++ | ++ | 4L | +++ | ++ | 4m | − | − |
| 51a | + | + | 4g | ++ | ++ | 41b | − | − |
| 51b | +++ | +++ | 19e | +++ | ++ | 26 | − | − |
| 51c | +++ | +++ | 4o | +++ | +++ | 44a | − | − |
| 51d | +++ | + | 4i | +++ | +++ | 23d | − | − |
| 51e | +++ | + | 4p | +++ | +++ | 44b | − | − |
| 51f | +++ | + | 4f | ++ | +++ | 49d | +++ | ++ |
| 51g | ++ | + | 4h | +++ | +++ | 49e | +++ | ++ |
| 51h | +++ | +++ | 4e | ++ | +++ | 49f | ++ | +++ |
| 51i | + | + | 4b | +++ | +++ | 49g | +++ | ++ |
| 51j | +++ | +++ | 4j | +++ | ++ | 51m | +++ | ++ |
| 51k | ++ | ++ | 19d | ++ | +++ | 61a | + | + |
| 59a | + | + | 4c | ++ | ++ | 61b | ++ | ++ |
| 59b | + | + | 47 | + | ++ | 61c | + | + |
| 57a | + | + | 4a | − | +++ | 61d | +++ | ++ |
| 57b | + | + | 4d | +++ | − | 61e | + | ++ |
| 53a | + | + | 4k | ++ | + | 61f | + | ++ |
| 53b | + | + | 19a | − | ++ | 61g | ++ | ++ |
| 55a | + | + | 23a | − | +++ | 61h | ++ | ++ |
| 55b | + | + | 19c | − | ++ | 23c | − | ++ |

KEY:
+++ MIC 1-10 μg/mL;
++ MIC 10-32 μg/mL;
+ 32-100 μg/mL;
− >100 μg/mL.
TFA = trifluoroacetate;
AcOH = acetate

In addition, three compounds were tested against methicillin-resistant staphylococcus aureus (MRSA) strains MRSA-III (ST239) and EMRSA-15.

| | MIC against MRSA- | |
|---|---|---|
| Compound | III | MIC against MRSA-1 |
| 49a | +++ | +++ |
| 19e | +++ | +++ |
| 4L | +++ | +++ |

KEY:
+++ MIC 1-10 μg/mL

1. A compound comprising:
Formula I 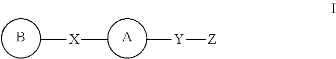
wherein
A is an optionally substituted aryl or heteroaryl group;
B is an optionally substituted aryl or heteroaryl group;
X is a group —CH₂O—;
Y is selected from methylene, C═O, C(R⁵)═N, and —CH₂O—; and
Z comprises a group: 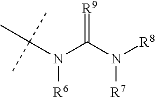
wherein R⁵ is selected from a group consisting of H and (C1-C6)alkyl;
R⁶, R⁷, and R⁸ are independently selected from a group consisting of H, (C1-C6)alkyl, CN, NO₂, (C1-C6)acyl, NH₂, NH(C1-C6)alkyl, N((C1-C6)alkyl)₂, and (C1-C6)alkoxyalkyl;
R⁹ is selected from a group consisting of O, S, SO, SO₂, NR¹⁰, and CR¹¹R¹²;
R¹⁰ is selected from a group consisting of H, (C1-C6)alkyl, CN, NO₂, (C1-C6)acyl, NH₂, NH(C1-C6)alkyl, N((C1-C6)alkyl)₂, C(═NH)NH₂, and (C1-C6) alkoxyalkyl;
R¹¹ and R¹² are independently selected from a group consisting of H, (C1-C6)alkyl, CN, and NO₂;
or, taken together with the atoms to which they are attached, R⁶ and R⁷, R⁷ and R⁸, R⁸ and R¹⁰, R⁶ and R¹⁰, R⁶ and R¹¹, or R⁸ and R¹¹ form an optionally substituted 3 to 6 membered heteroaryl or heterocyclyl ring optionally containing 1 or 2 further heteroatoms selected from a group consisting of O, N and S;
or, taken together with the atoms to which they are attached, R⁶, R⁷, R⁸, R¹⁰, or R¹¹ form an optionally substituted 3 to 6 membered heteroaryl or heterocyclyl ring fused to ring A,
or a pharmaceutically active salt or N-oxide thereof.

2. The compound of claim 1, wherein A is an aryl group selected from a group consisting of phenyl, thiazolyl, pyridyl, imidazolyl, and benzothiazole.

3. The compound of claim 1 wherein A is a phenyl group, optionally substituted with from one to three groups independently selected from a group consisting of halo, C1-C6 alkyl, C1-C6 haloalkyl, C1-C6 alkoxy, C1-C6 haloalkoxy, nitro, cyano and C1-C6 thioalkyl.

4. The compound of claim 3, wherein the compound has the formula 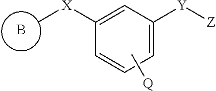
wherein Q is optionally present and represents from one to three groups independently selected from a group consisting of halo, C1-C6 alkyl, C1-C6 haloalkyl, C1-C6 alkoxy, C1-C6 haloalkoxy, nitro, cyano and C1-C6 thioalkyl.

5. The compound of claim 1, wherein B is an aryl group selected from a group consisting of phenyl, thiazolyl, pyridyl, and benzothiazole.

6. The compound of claim 5, wherein B is a phenyl group, optionally substituted with from one to three groups independently selected from a group consisting of halo, C1-C6 alkyl, C1-C6 haloalkyl, C1-C6 alkoxy, C1-C6 haloalkoxy, nitro, cyano and C1-C6 thioalkyl.

7. The compound of claim 1, wherein Y is CH═N.

8. The compound of claim 1, wherein R⁹ is NR¹⁰, with R¹⁰ as defined in claim 1.

9. The compound of claim 8, wherein R¹⁰ is H.

10. The compound of claim 1, wherein R⁶, R⁷ and R⁸ are all H.

11. The compound of claim 1 which is one of 1-[({3-[(2,3-dichlorophenyl)methoxy]phenyl}methylidene)amino]guanidine; 1-{[(3-{[2-chloro-3-(trifluoromethyl)phenyl]methoxy}phenyl)methylidene]amino} guanidine; 7-[(2,3-dichlorophenyl)methoxy]-1,2,3,4-tetrahydroisoquinoline-2-carboximidamide; 5-[(2,3-dichlorophenyl)methoxy]-1,2,3,4-tetrahydroisoquinoline-2-carboximidamide; 1-({3-[(2,3-dichlorophenyl)methoxy]phenyl}methyl)-1-methylguanidine; 1-({3-[(2,3-dichlorophenyl) methoxy]phenyl}methyl)-1-(2-methoxyethyl) guanidine; 3-({3-[(2,3-dichlorophenyl)methoxy] phenyl}methyl)-1-methylguanidine; (E)-Amino(2-(2-chloro-3-((2-chloro-3-methoxybenzyl) oxy)benzylidene) hydrazinyl)methanimine; (E)-Amino(2-(3-(benzyloxy)-2-chlorobenzylidene) hydrazinyl)methanimine; (E)-Amino(2-(2-chloro-3-((4-chlorobenzyl)oxy)benzylidene) hydrazinyl)methanimine; (E)-Amino(2-(2-chloro-3-((2,3-dichlorobenzyl)oxy)benzylidene) hydrazinyl)methanimine; (E)-Amino(2-(2-chloro-3-((2-chloro-3-(trifluoromethyl)benzyl)oxy) benzylidene) hydrazinyl)methanimine; (E)-Amino(2-(2-chloro-3-((2,4-dichlorobenzyl) oxy)benzylidene)hydrazinyl) methanimine(E)-Amino(2-(2-chloro-3-((2,5-dichlorobenzyl)oxy) benzylidene)hydrazinyl) methanimine; (E)-Amino(2-(2-chloro-3-((3,4-dichlorobenzyl)oxy) benzylidene)hydrazinyl) methanimine; (E)-Amino(2-(2-chloro-3-((2,3,5-trichlorobenzyl)oxy) benzylidene)hydrazinyl) methanimine; (E)-Amino(2-(2-chloro-3-((3-(trifluoromethyl)benzyl) oxy)benzylidene)hydrazinyl) methanimine; (E)-Amino(2-(2-chloro-3-((4-(trifluoromethyl) benzyl)oxy)benzylidene)hydrazinyl) methanimine; 1-[(3-{[2-chloro-3-(trifluoromethyl)phenyl]methoxy}phenyl)methyl]guanidine; 1-({3-[(2,3-dichlorophenyl)methoxy]phenyl}methyl)guanidine; 1-(4-((2,3-dichlorobenzyl)oxy)benzyl) guanidine; 1-({3-[(2,3-dichlorophenyl)methoxy]-2,6-difluorophenyl}methyl)guanidine; 1-[(3-{[3-(trifluoromethyl)phenyl]methoxy}phenyl)methyl]guanidine; 1-[(2,6-difluoro-3-{[3-(trifluoromethyl)phenyl]methoxy}phenyl)methyl]guanidine; 1-({3-[(2,5-dichlorophenyl) methoxy]phenyl}methyl)guanidine; 1-[(3-{[4-(trifluoromethyl)phenyl]methoxy}phenyl)methyl]guanidine; 1-({3-[(3,4-dichlorophenyl)methoxy]phenyl}methyl)guanidine; 1-({3-[(4-chlorophenyl)methoxy]phenyl}methyl)guanidine; 1-({3-[(4-bromophenyl)methoxy] phenyl}methyl)guanidine; 1-(4-((3,4-dichlorobenzyl)oxy)benzyl)guanidine; 1-({3-[(3-chlorophenyl)methoxy]phenyl}methyl)guanidine; 3-(benzyloxy)-N-carbamimidoylbenzamide; 1-{[3-(benzyloxy)phenyl]methyl}guanidine; 1-({3-[(2,4-dichlorophenyl)methoxy]phenyl} methyl)guanidine; 1-({3-[(4-fluorophenyl)methoxy]phenyl}methyl)guanidine; 1-(4-(benzyloxy)benzyl)guanidine; 1-[4-(benzyloxy)phenyl]guanidine; 1-(4-((3-chlorobenzyl) oxy)benzyl)guanidine; 1-{4-[(3-chlorophenyl)methoxy]phenyl}guanidine; 1-({3-[(4-chlorophenyl)methoxy]-4-methoxyphenyl}methyl)guanidine; 1-(4-((4-chlorobenzyl)oxy) benzyl)guanidine; tert-butyl N-[7-(4-tert-butylphenyl)-1,2,3,4-tetrahydroisoquinoline-2-carboximidoyl]carbamate; 1-{4-[(4-chlorophenyl)methoxy]phenyl}guanidine; 1-({3-[(3-chlorophenyl)methoxy]-4-methoxyphenyl}methyl)guanidine; 1-[(3-phenoxyphenyl)methoxy] guanidine; 4-(4-chlorophenyl)piperazine-1-carboximidamide; 1-(5,6,7,8-tetrahydronaphthalen-1-yl)guanidine; 4-(3-methoxyphenyl)piperazine-1-carboximidamide; (E)-Amino(2-(3-((4-chlorobenzyl)oxy)benzylidene)hydrazinyl)methanimine; (E)-Amino(2-(3-((4-(trifluoromethyl)benzyl)oxy)benzylidene) hydrazinyl) methanimine; (E)-Amino(2-(3-((3-chlorobenzyl)oxy)benzylidene)hydrazinyl)methanimine; (E)-Amino(2-(3-((4-(trifluoromethyl)benzyl)oxy)benzylidene)hydrazinyl) methanimine; (E)-2-(3-((3,4-dichlorobenzyl)oxy)-4-methoxybenzylidene)hydrazine-1-carboximidamide; (E)-Amino(2-(2-chloro-3-((3-chlorobenzyl)oxy)benzylidene)hydrazinyl) methanimine; or a pharmaceutically active salt or N-oxide thereof.

12. A pharmaceutical composition comprising a compound of formula I as defined in claim 1, and a pharmaceutically acceptable vehicle.

13. The pharmaceutical composition of claim 12, further comprising one or more further antibiotics selected from a group consisting of macrolide antibiotics, β-lactam antibiotics, tetracycline antibiotics, and quinolone antibiotics.

14. The pharmaceutical composition of claim 13 wherein the further antibiotic is selected from a group consisting of azithromycin, clarithromycin, dirithromycin, erythromycin, roxithromycin, telithromycin, CarbomycinA, josamycin, kitasamycin, midecamicine, oleandomycin, spiramycin, tylosin, troleandomycin, aztreonam, imipenem, meropenem, ertapenem, doripenem, panipenem/betamipron, biapenem, PZ-601, cefixime, cefdinir, cefditoren, cefoperazone, cefotaxime, cefpodoxime, ceftazidime, ceftibuten, ceftizoxime, ceftriaxone, cefepime, demeclocycline, doxycycline, minocycline, oxytetracycline, tetracycline, ciprofloxacin, enoxacin, gatifloxacin, levofloxacin, lomefloxacin, moxifloxacin, norfloxacin, ofloxacin, and trovafloxacin, preferably ceftazidime, imipenem/cilastatin, meropenem, aztreonam, oxytetracycline, azithromycin, clarithromycin, dirithromycin, erythromycin, roxithromycin, spiramycin, and ciprofloxacin.

15. The compound of claim 1 for use as a medicament.

16. The compound of claim 1 for use in the treatment or prophylaxis of a bacterial infection in an animal.

17. The compound of claim 16 wherein the animal is a mammal.

18. The compound of claim 17 wherein the mammal is a human.

19. The compound of claim 16 wherein the bacterial infection is a Gram-negative bacterial strain infection.

20. The compound of claim 19, wherein the Gram-negative bacterial strain is selected from the group consisting of Escherchia coli, Caulobacter crescentus, Pseudomonas aeruginosa, Agrobacterium tumefaciens, Branhamella catarrhalis, Citrobacter diversus, Enterobacter aerogenes, Enterobacter cloacae, Enterobacter sakazakii, Enterobacter asburiae, Pantoea agglomerans, Klebsiella pneumoniae, Klebsiella oxytoca, Klebsiella rhinoscleromatis, Proteus mirabilis, Salmonella typhimurium, Salmonella enteriditis, Serratia marcescens, Shigella sonnei, Neisseria gonorrhoeae, Acinetobacter baumannii, Acinetobacter calcoaceticus, Acinetobacter lwoffi, Salmonella enteriditis, Fusobacterium nucleatum, Veillonella parvula, Bacteroides forsythus, Actinobacillus actinomycetemcomitans, Aggregatibacter actinomycetemcomitans, Porphyromonas gingivalis, Helicobacter pylori, Francisella tularensis, Yersinia pestis, Borrelia burgdorferi, Neisseria meningitidis and Haemophilus influenzae.

21. The compound of claim 16 wherein the bacterial infection is a Gram-positive bacterial strain infection.

22. The compound of claim 21 wherein the Gram-positive bacterial strain is selected from the group consisting of Staphylococcus aureus, Staphylococcus epidermidis, Staphylococcus saprophyticus, Streptococcus pyogenes, Streptococcus faecalis, Enterococcus faecalis, Enterococcus faecium, Bacillus subtilis, Micrococcus luteus, Mycobacterium tuberculosis, Bacillus anthracis, Bacillus cereus, Clostridium difficile, Propionibacterium acnes, Streptococcus mutans, Actinomyces viscosus, Actinomyces naeslundii, Streptococcus sanguis, Streptococcus pneumoniae and Streptococcus salivarius.

23. The compound of claim 16 wherein the bacterial infection is a multiple drug-resistant bacterial strain infection.

24. The compound of claim 23 wherein the multiple drug-resistant bacterial strain is selected from the group consisting of methicillin-resistant Staphylococcus aureus, vancomycin-resistant Enterococcus, multiple drug-resistant tuberculosis, and multidrug-resistant Clostridium difficile.

25. A method of treatment or prophylaxis of a bacterial infection in an animal comprising administering to an animal in need thereof a therapeutically effective dose of the compound of claim 1.

26. The composition of claim 12 for use as a medicament.

27. The composition of claim 12 for use in the treatment or prophylaxis of a bacterial infection in an animal.

28. A method of treatment or prophylaxis of a bacterial infection in an animal comprising administering to an animal in need thereof a therapeutically effective dose of the composition of claim 12.
